(19) United States
(12) Patent Application Publication
(10) Pub. No.: US 2026/0120813 A1
(43) Pub. Date: Apr. 30, 2026

(54) METHODS AND SYSTEMS FOR SINGLE MOLECULE TRACKING IN CELL-FREE SYSTEMS

(71) Applicant: EIKON THERAPEUTICS, INC., Millbrae, CA (US)

(72) Inventors: Markus Seeger, Millbrae, CA (US); Daniel Anderson, Millbrae, CA (US); Stephanie Johnson, Millbrae, CA (US); Zhengjiang Zhang, Millbrae, CA (US)

(73) Assignee: EIKON THERAPEUTICS, INC., Millbrae, CA (US)

(21) Appl. No.: 19/431,552

(22) Filed: Dec. 23, 2025

Related U.S. Application Data
(63) Continuation of application No. PCT/US2024/035637, filed on Jun. 26, 2024.
(60) Provisional application No. 63/523,356, filed on Jun. 26, 2023.
(60) Provisional application No. 63/613,704, filed on Dec. 21, 2023.

(51) Int. Cl. G16C 20/10; G01N 33/58

(52) U.S. Cl. CPC G16C 20/10; G01N 33/582

(57) ABSTRACT

The present disclosure relates to single molecule tracking methods, including high throughput methods, for analyzing molecules in cell-free samples. The present disclosure further provides systems for performing such methods.

### CROSS-REFERENCE TO RELATED APPLICATIONS

The application is a Continuation application of International Application No. PCT/US2024/035637, filed Jun. 26, 2024, which claims priority to U.S. Provisional Application No. 63/523,356, filed Jun. 26, 2023, and U.S. Provisional Application No. 63/613,704, filed Dec. 21, 2023, the contents of each of which are incorporated herein by reference in their entireties.

### TECHNICAL FIELD

The subject matter described herein relates to methods for analyzing single molecules in cell-free systems.

### BACKGROUND

The movement of molecules within a cell-free system is profoundly influenced by the interactions of those molecules with their environment. This environment includes, but is not limited to, the solution in which the molecules are distributed, other molecules located within the solution, as well as physical conditions. Monitoring the movement of target molecules can thus provide biophysical information underlying those environmental interactions and effects on the targeted molecules. Single molecule tracking (SMT) is one method for capturing single molecule movement, e.g., as a reporter of a target molecule's conformation, interactions, and/or identity. In SMT, a molecule, e.g., a fluorescently-labelled molecule, is imaged at high spatiotemporal resolution over time and its movement is analyzed on a single-molecule level. To date, however, the application of SMT techniques to cell-free systems has been limited in scale, sensitivity, and success.

### SUMMARY OF THE INVENTION

The present disclosure relates to single molecule methods, including, e.g., high throughput methods, for analyzing target molecules in cell-free samples. The present disclosure further provides systems for performing such methods.

In a first aspect, the present disclosure is directed to methods, e.g., high throughput methods, for identifying interactions between a test molecule and a target molecule comprising: (a) contacting a cell-free sample comprising a plurality of the target molecule with a plurality of the test molecule; (b) tracking a plurality of the target molecules over time to provide a plurality of spatiotemporal trajectories and/or measurements of rotational motion; (c) analyzing the plurality of spatiotemporal trajectories and/or measurements of rotational motion to determine the movement of the target molecule in the presence of the test molecule; and (d) comparing the target molecule movement obtained in (c) with the movement of a reference target molecule, wherein the reference target molecule movement is the movement of the target molecule in the absence of the test molecule; wherein a change in the target molecule movement compared to the reference target molecule movement indicates an interaction between the target molecule and the test molecule. In certain embodiments, a decrease in a target molecule's movement compared to a reference target molecule's movement indicates an interaction between the target molecule and the test molecule. In certain embodiments, an increase in the target molecule's movement compared to the reference target molecule's movement indicates an interaction between the target molecule and the test molecule. In certain embodiments, the duration and/or reversibility of the target molecule's movement change compared to the reference target molecule's movement change indicates an interaction between the target molecule and the test molecule.

In certain embodiments directed to identifying interactions between a test molecule and a target molecule, a determination of molecule's movement can be quantified by analysis of spatiotemporal trajectories. Movement characterized in this way can also include, but not be limited to, measurements of diffusion coefficient. For example, the measurement can be of the diffusion coefficient maximum likelihood estimator, defined as an estimate of the maximum likelihood diffusion coefficient for the plurality of spatiotemporal trajectories under a single-state diffusion model with constant localization error. In certain embodiments, spatiotemporal trajectories can be used to estimate the fraction of target molecules in multiple dynamical diffusive states, e.g., different diffusive states, using machine learning methods such as variational Bayesian inference. Such methods include, but are not limited to, state arrays. In certain embodiments, molecule movement may be measured through analysis of the product of the link-generating algorithm. Movement characterized in this way can include, but not be limited to, the mean posterior diffusion coefficient, the mean of the posterior probability distribution of coefficients from a probabilistic linking algorithm. Movement characterized in this way can include, but not be limited to, the geometric mean posterior diffusion coefficient, the mean of the log-scaled posterior probability distribution of coefficients from a probabilistic linking algorithm. In certain embodiments, a molecule's movement can be measured through model-dependent analysis of the plurality of spatiotemporal trajectories. In certain embodiments, a determination of a molecule's movement can be quantified by calculating a jump length distribution. For example, for any given set of protein displacements between one timepoint and a subsequent timepoint, a histogram can be constructed of the probability of each of the displacement lengths (“jump lengths”). Quantiles of this distribution can be used to describe the motion of the molecule. In certain embodiments the quantile used is the median of the jump length distribution. In certain embodiments, the quantile used is the 3rd quartile of the jump length distribution. In certain embodiments, a molecule's movement may be quantified by measurements of the mean squared displacement as defined by the average of the square of all displacements in a spatiotemporal trajectory, averaged over the plurality of spatiotemporal trajectories. Movement characterized in this way can also include, but not be limited to, measurements of the spatiotemporal trajectory length or distribution of spatiotemporal trajectory lengths. Movement characterized in this way can also include, but not be limited to, measurements of the mean radius of gyration, as defined by the root mean square distance of all coordinates in a spatiotemporal trajectory from the center of mass of the set of points contained in the spatiotemporal trajectory, averaged over the plurality of spatiotemporal trajectories. Movement characterized in this way can also include, but not be limited to, measurements of the mean bond angle, defined by the angle formed from three sequential spatial coordinates averaged over the plurality of spatiotemporal trajectories.

In certain embodiments, a determination of a molecule's movement can be quantified by analysis of measurements of rotational motion. Movement characterized in this way can include, but is not limited to, anisotropy decay time.

In certain embodiments, movement can be characterized by determining the number and wavelength of conjugated fluorescent labels. In certain embodiments, movement can be characterized by determining the polarization of conjugated fluorescent labels.

In certain embodiments, a determination of a molecule's interaction can be quantified by analysis of measurement of the spatial extent of the detection. In certain embodiments, interactions characterized in this way can include, but not limited to, measurement of oligomerization. In certain embodiments, oligomerization processes analyze one or more detection channels to analyze, but not limited to, the number and heterogeneity of monomers forming oligomers.

In certain embodiments directed to identifying interactions between a test molecule and a target molecule, e.g., where movement is determined using a change in a diffusion coefficient relative to a reference target molecule diffusion coefficient, the change in the diffusion coefficient compared to the reference target molecule diffusion coefficient is at least about 0.001%, at least about 0.005%, at least 0.01%, at least about 0.5%, at least about 1%, at least about 1.5%, at least about 2% or at least about 3%, at least about 4%, at least about 5%, at least about 6%, at least about 7%, at least about 8%, at least about 9%, or at least about 10%.

In certain embodiments directed to identifying interactions between a test molecule and a target molecule, the change in a target molecule's movement compared to a reference target molecule's movement indicates a direct interaction between the target molecule and the test molecule. In certain embodiments, the change in the target molecule's movement compared to the reference target molecule's movement indicates an indirect interaction between the target molecule and the test molecule. In certain embodiments, the interaction between the target molecule and the test molecule results in a conformational change in the target molecule. In certain embodiments, the interaction between the target molecule and the test molecule results in an increase in the mass of the target molecule. In certain embodiments, the interaction between the target molecule and the test molecule results in a decrease in the mass of the target molecule. In certain embodiments, the interaction between the target molecule and the test molecule results in a change in the target molecule's temperature stability. In certain embodiments, the interaction between the target molecule and the test molecule results in oligomerization processes. In certain embodiments, a plurality of target molecules are conjugated to a plurality of fluorescent labels. In certain embodiments, the interaction between the target molecule and the test molecule is reversible. In certain embodiments, the interaction between the target molecule and the test molecule is irreversible.

In an another aspect, the present disclosure provides methods, e.g., high throughput methods, for determining the form of a target molecule comprising: (a) tracking a plurality of the target molecule over time in a cell-free sample to provide a plurality of spatiotemporal trajectories and/or measurements of rotational motion; (b) analyzing the plurality of spatiotemporal trajectories and/or measurements of rotational motion to determine the target molecule's movement; and (c) comparing the target molecule's movement obtained in (b) with a reference target molecule's movement, wherein the reference target molecule's movement is the movement of one form of the target molecule; wherein: (i) a change in the target molecule's movement compared to the reference target molecule's movement indicates that the target molecule is of a different form than the reference target molecule; or (ii) no change in the target molecule's movement compared to the reference target molecule's movement indicates that the target molecule is of the same form of the target molecule. In certain embodiments, the duration and/or reversibility of the target molecule's movement change compared to the duration and/or reversibility of the reference target molecule's movement change indicates that the target molecule is of a different form than the reference target molecule. In certain embodiments, the different forms of the target molecule can be of the same chemical composition but in different conformations. In certain embodiments, one form of the target molecule can be a product of alternatively splicing. In certain embodiments, one form of the target molecule is a post-translationally modified form of the target molecule. In certain embodiments, one form of the target molecule is a wild type form of the target molecule. In certain embodiments, one form of the target molecule is a mutant form of the target molecule. In certain embodiments, one form of the target molecule is a homolog, ortholog or paralog of the target molecule.

In certain embodiments, the change in movement associated with determining the form of a target molecule is a change in (a) diffusion coefficient obtained from a maximum likelihood estimator; (b) geometric mean posterior diffusion coefficient; (c) median of the jump length distribution; (d) 3rd quartile of the jump length distribution; (e) median radius of gyration; (f) mean posterior diffusion coefficient; (g) mean squared displacement; (h) median bond angle; (i) spatiotemporal trajectory length; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.

In certain embodiments directed to determining the form of a target molecule, e.g., where movement is determined using a change in a diffusion coefficient relative to a reference target molecule's diffusion coefficient, the change in diffusion coefficient is at least about 0.001%, at least about 0.005%, at least about 0.01%, at least about 0.05%, at least about 0.1%, at least about 0.5%, at least about 1%, at least about 1.5%, at least about 2% or at least about 3%, at least about 4%, at least about 5%, at least about 6%, at least about 7%, at least about 8%, at least about 9%, or at least about 10%.

In a further aspect, the present disclosure provides methods, e.g., high throughput methods, for identifying a test molecule that can distinguish between at least two target molecules. In certain embodiments, the methods include: (a) contacting a cell-free sample comprising a plurality of a first target molecule with a plurality of the test molecule; (b) tracking a plurality of the first target molecule over time to provide a plurality of spatiotemporal trajectories and/or measurements of rotational motion; (c) analyzing the plurality of spatiotemporal trajectories and/or measurements of rotational motion to determine the first target molecule's movement in the presence of the test molecule; (d) contacting a cell-free sample comprising a plurality of a second target molecule with a plurality of the test molecule; (e) tracking a plurality of the second target molecule over time to provide a plurality of spatiotemporal trajectories and/or measurements of rotational motion; (f) analyzing the plurality of spatiotemporal trajectories and/or measurements of rotational motion to determine the movement of the second target molecule in the presence of the test molecule; and (g) comparing the target molecule's movement obtained in (c) and (f); wherein a change in the movement of the first and second target molecule indicates that the test molecule can distinguish between the two target molecules.

In certain embodiments, the change in movement associated with identifying a test molecule that can distinguish between at least two target molecules is a change in (a) diffusion coefficient obtained from a maximum likelihood estimator; (b) geometric mean posterior diffusion coefficient; (c) median of the jump length distribution; (d) 3rd quartile of the jump length distribution; (e) median radius of gyration; (f) mean posterior diffusion coefficient; (g) mean squared displacement; (h) median bond angle; (i) spatiotemporal trajectory length; or (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.

In certain embodiments, a change in movement of the first and second target molecule indicates that the test molecule can distinguish between the two target molecules. In certain embodiments, the two target molecules are not related. In certain embodiments, the two target molecules are related. In certain embodiments where the two target molecules are related, the first target molecule and the second target molecule are the same or different forms of the target molecule. In certain embodiments where the two target molecules are related, one form of the target molecule is a wild type form of the target molecule. In certain embodiments where the two target molecules are related, one form of the target molecule is a mutant form of the target molecule. In certain embodiments, one form of the target molecule is a post-translationally modified form of the target molecule. In certain embodiments where the two target molecules are related, the first target molecule and the second target molecule are homologs, orthologs or paralogs. In certain embodiments where the two target molecules are related, the first target molecule and the second target molecule are different species of the target molecule. In certain embodiments where the two target molecules are related, the first target molecule and the second target molecule are different splicing isoforms of the target molecule. In certain embodiments where the two target molecules are related, the first target molecule and the second target molecule are the same or different conformational states of the target molecule. In certain embodiments, distinguishing of the first and second target molecule is achieved by difference in their fluorescent labels. In certain embodiments, more than two target molecules are analyzed, e.g., a third target molecule is analyzed. For example, but not by way of limitation, the method can further include contacting a cell-free sample comprising a plurality of a third target molecule with a plurality of the test molecule; tracking a plurality of the third target molecules over time to provide a third plurality of spatiotemporal trajectories and/or measurements of rotational motion; analyzing the third plurality of spatiotemporal trajectories and/or measurements of rotational motion to determine the movement of the third target molecules in the presence of the test molecule; and comparing the third target molecule's movement to the movements obtained in (c) and (f); wherein a change in the movement of the third target molecule relative to the first and second target molecules indicates that the test molecule can distinguish between the three target molecules.

In a further aspect, the present disclosure provides methods, e.g., high throughput methods, for identifying test molecules that bind to a target molecule and cause a conformational change in the target molecule. In certain embodiments, the methods comprise: (a) contacting cell-free samples comprising a plurality of the target molecule with a plurality of test molecules, where each sample is contacted with a different test molecule; (b) tracking a plurality of the target molecules in each sample over time to provide a plurality of spatiotemporal trajectories and/or measurements of rotational motion; (c) analyzing the plurality of spatiotemporal trajectories and/or measurements of rotational motion to determine the target molecule's movement in the presence of the test molecules; and (d) comparing the target molecule's movement obtained in step (c) with a reference target molecule's movement, wherein the reference target molecule's movement is the movement of the target molecule in the presence of a test molecule that does not induce a conformational change in the target molecule; wherein a change in the target molecule's movement compared to the reference target molecule's movement indicates an interaction between the target molecule and the test molecule that induces a conformational change in the target molecule. In certain embodiments, the duration and/or reversibility of the target molecule's movement change compared to the duration and/or reversibility of the reference target molecule's movement change indicates an interaction between the target molecule and the one or more test molecules that induce a conformational change in the target molecule. In certain embodiments, the conformational change in the target molecule is associated with a change in the target molecule's temperature stability.

In certain embodiments, the change in movement associated with identifying test molecules that bind to a target molecule and cause a conformational change in the target molecule is a change in (a) diffusion coefficient obtained from a maximum likelihood estimator; (b) geometric mean posterior diffusion coefficient; (c) median of the jump length distribution; (d) 3rd quartile of the jump length distribution; (e) median radius of gyration; (f) mean posterior diffusion coefficient; (g) mean squared displacement; (h) median bond angle; (i) spatiotemporal trajectory length; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.

In certain embodiments directed to identifying test molecules that bind to a target molecule and cause a conformational change in the target molecule, e.g., where movement is determined using a change in a diffusion coefficient relative to a reference target molecule's diffusion coefficient, the change in the diffusion coefficient compared to the reference target molecule diffusion coefficient is at least about 0.001%, at least about 0.005%, at least about 0.01%, at least about 0.05%, is at least about 0.1%, at least about 0.5%, at least about 1%, at least about 1.5%, at least about 2% or at least about 3%, at least about 4%, at least about 5%, at least about 6%, at least about 7%, at least about 8%, at least about 9% or at least about 10%.

In another aspect, the present disclosure provides methods, e.g., high throughput methods, for determining a dose response of a target molecule to a test molecule comprising: (a) contacting a plurality of cell-free samples comprising a plurality of the target molecule with the test molecule, where plurality of cell-free samples are contacted with a range of test molecule doses; (b) tracking a plurality of the target molecules over time in the presence of the range of doses of the test molecule to provide a plurality of spatiotemporal trajectories and/or measurements of rotational motion; (c) analyzing the plurality of spatiotemporal trajectories and/or measurements of rotational motion of the target molecules in the presence of the range of doses of the test molecule to determine the target molecule's movement at each dose of test molecule; and (d) comparing the target molecule's movement obtained at different test molecule doses in step (c) to determine the dose response of the target molecule to the test molecule.

In certain embodiments, the change in movement associated with determining a dose response of a target molecule to a test molecule is a change in (a) diffusion coefficient obtained from a maximum likelihood estimator; (b) geometric mean posterior diffusion coefficient; (c) median of the jump length distribution; (d) 3rd quartile of the jump length distribution; (e) median radius of gyration; (f) mean posterior diffusion coefficient; (g) mean squared displacement; (h) median bond angle; (i) the spatiotemporal trajectory length; or (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.

In certain embodiments directed to determining a dose response of a target molecule to a test molecule, e.g., where movement is determined using a change in a target molecule's diffusion coefficient relative to the diffusion coefficient of the target molecule at each test molecule dose, the change in the target molecule's diffusion coefficient compared to the diffusion coefficient of the target molecule at each test molecule dose is at least about 0.001%, at least about 0.005%, at least about 0.01%, at least about 0.05%, is at least about 0.1%, at least about 0.5%, at least about 1%, at least about 1.5%, at least about 2% or at least about 3%, at least about 4%, at least about 5%, at least about 6%, at least about 7%, at least about 8%, at least about 9% or at least about 10%.

In a further aspect, the present disclosure provides methods, e.g., high throughput methods, for determining a difference in dose response to a test molecule by two target molecules comprising: (a) contacting a first plurality of cell-free samples comprising a plurality of the first target molecule with the test molecule, where first plurality of cell-free samples are contacted with a range of test molecule doses; (b) tracking a plurality of the target molecules over time in the presence of the range of doses of the test molecule to provide a first plurality of spatiotemporal trajectories and/or measurements of rotational motion; (c) analyzing the plurality of spatiotemporal trajectories and/or measurements of rotational motion of the target molecules in the presence of the range of doses of the test molecule to determine the first target molecule's movement at each dose of test molecule; (d) comparing the target molecule's movement obtained at different test molecule doses in (c) to determine the dose response of the target molecule to the test molecule; (e) repeating steps (a)-(d) with a second target molecule to determine the dose response of the second target molecule to the test molecule; and (f) comparing the dose response of the first target molecule to the dose response of the second target molecule to determine the difference in the response of the first and second target molecules to the test molecule. In certain embodiments, the distinguishing of the first and second target molecule is achieved by difference in their fluorescent labels. In certain embodiments, more than two target molecules are analyzed, e.g., three or more target molecules are analyzed.

In certain embodiments, the change in movement associated with determining a difference in dose response to a test molecule by two target molecules is a change in (a) diffusion coefficient obtained from a maximum likelihood estimator; (b) geometric mean posterior diffusion coefficient; (c) median of the jump length distribution; (d) 3rd quartile of the jump length distribution; (e) median radius of gyration; (f) mean posterior diffusion coefficient; (g) mean squared displacement; (h) median bond angle; (i) the spatiotemporal trajectory length; or (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.

In certain embodiments directed to determining a difference in dose response to a test molecule by two target molecules, the first and second target molecules are not related. In certain embodiments, the first and second target molecules are related. In certain embodiments where the first and second target molecules are related, the first target molecule and the second target molecule are the same or different forms of the target molecule. In certain embodiments where the first and second target molecules are related, one form of the target molecule is a wild type form of the target molecule. In certain embodiments where the first and second target molecules are related, one form of the target molecule is a mutant form of the target molecule. In certain embodiments where the first and second target molecules are related, one form of the target molecule is a post-translationally modified form of the target molecule. In certain embodiments where the first and second target molecules are related, the first target molecule and the second target molecule are homologs, orthologs, or paralogs. In certain embodiments where the first and second target molecules are related, the first target molecule and the second target molecule are different species of the target molecule. In certain embodiments where the first and second target molecules are related, the first target molecule and the second target molecule are different splicing isoforms of the target molecule. In certain embodiments where the first and second target molecules are related, the first target molecule and the second target molecule are the same or different conformational states of the target molecule. In certain embodiments, the distinguishing of the first and second target molecule is achieved by difference in their fluorescent labels. In certain embodiments, more than two target molecules are analyzed.

In certain embodiments directed to determining a difference in dose response to a test molecule by two target molecules, the target molecule is an organic molecule of less than about 1 kDa. In certain embodiments, the target molecule is selected from the group consisting of a peptide, a protein domain, a protein, a nucleic acid polymer, a carbohydrate, and a lipid. In certain embodiments, the target molecule is selected from the group consisting of a glycoprotein, glycolipid, oligosaccharide, polysaccharide, and lipid micelle. In certain embodiments, the target molecule is an entity of multiple components (e.g., at least two, at least three, at least four or at least five components) non-covalently bound together by interactions including but not limited to hydrophobic forces, electrostatic forces, ionic forces, hydrogen forces, and Van der Waals forces. In certain embodiments, the target molecule is an entity of multiple components covalently bound together. In certain embodiments, the target molecule is a multi-subunit protein or protein complex composed of multiple subunits. In certain embodiments, the target molecule is a protein composed of one or more polypeptides and one or more ligands. In certain embodiments, the target molecule is an exosome composed of proteins and lipids and other biological molecules. In certain embodiments, the target molecule is a virus, viroid, phage, and other biological particle. In certain embodiments, the protein is selected from the group consisting of an antibody, a receptor, and an enzyme. In certain embodiments, the protein comprises a disordered domain. In certain embodiments, the protein does not comprise a structured domain. In certain embodiments, the peptide is a ligand. In certain embodiments, the target molecule is a nanomaterial. In certain embodiments, the target molecule is a synthetic polymer. In certain embodiments, the target molecule is labeled. In certain embodiments, the target molecule is fluorescently labeled. In certain embodiments, the target molecule is labeled with organic fluorophores or inorganic fluorescent particles. In certain embodiments, the target molecule is labeled with a fluorescent protein. In certain embodiments, the target molecule is labeled by conjugation of a synthetic nanomaterial or polymer. In certain embodiments, the target molecule is purified. In certain embodiments, the target molecule is a component of a mixture. In certain embodiments, the target molecule is a component of a mixture composed of a bacterial extract, cell extract, tissue extract, plant extract, or animal extract. In certain embodiments, the bacterial extract, cell extract, tissue extract, plant extract, or animal extract is a lysate. In certain embodiments, the target molecule is a component of a mixture composed of serum, blood, and other biological samples. In certain embodiments, the target molecule is a component of a mixture comprising a buffer and glycerol.

In a further aspect, the present disclosure provides high throughput methods, e.g., high throughput methods, for identifying an interaction between a test molecule and a target protein comprising a disordered domain comprising: (a) contacting a cell-free sample comprising a plurality of the target protein with a plurality of the test molecule; (b) tracking a plurality of the target protein over time to provide a plurality of spatiotemporal trajectories and/or measurements of rotational motion; (c) analyzing the plurality of spatiotemporal trajectories and/or measurements of rotational motion to determine the target protein's movement in the presence of the test molecule; and (d) comparing the target protein's movement obtained in (c) with a reference target protein's movement, wherein the reference target protein's movement is the movement of the target protein in the absence of the test molecule; wherein a change in the movement of the target protein comprising the disordered domain compared to the reference target protein's movement indicates an interaction between the target protein comprising the disordered domain and the test molecule. In certain embodiments, the duration and/or reversibility of the movement change of the target protein comprising the disordered domain compared to the duration and/or reversibility of the reference target protein's movement change indicates an interaction between the target molecule and the test molecule. In certain embodiments, the interaction between the target molecule and the test molecule is reversible. In certain embodiments, the interaction between the target molecule and the test molecule is irreversible.

In certain embodiments, the change in movement associated with identifying an interaction between a test molecule and a target protein comprising a disordered domain is a change in (a) diffusion coefficient obtained from a maximum likelihood estimator; (b) geometric mean posterior diffusion coefficient; (c) the median of the jump length distribution; (d) 3rd quartile of the jump length distribution; (e) median radius of gyration; (f) mean posterior diffusion coefficient; (g) mean squared displacement; (h) median bond angle; (i) the spatiotemporal trajectory length; or (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.

In certain embodiments directed to identifying an interaction between a test molecule and a target protein comprising a disordered domain, e.g., where movement is determined using a change in a diffusion coefficient relative to a reference target molecule diffusion coefficient, the change in the diffusion coefficient of the target protein comprising the disordered domain compared to the reference target protein diffusion coefficient is at least about 0.001%, at least about 0.005%, at least about 0.01%, at least about 0.05%, at least about 0.1%, at least about 0.5%, at least about 1%, at least about 1.5%, at least about 2% or at least about 3%, at least about 4%, at least about 5%, at least about 6%, at least about 7%, at least about 8%, at least about 9% or at least about 10%. In certain embodiments, the change in the diffusion coefficient of the target protein comprising the disordered domain compared to the reference target protein diffusion coefficient indicates a direct interaction between the target protein comprising the disordered domain and the test molecule. In certain embodiments, the change in the target protein diffusion coefficient compared to the reference target protein diffusion coefficient indicates an indirect interaction between the target protein comprising the disordered domain and the test molecule. In certain embodiments, the change in the diffusion coefficient of the target protein comprising the disordered domain compared to the reference target protein diffusion coefficient indicates a conformational change in the target protein. In certain embodiments, the conformational change in the target protein results in a change in the target protein's temperature stability. In certain embodiments, the change in the diffusion coefficient of the target protein comprising the disordered domain compared to the reference target protein diffusion coefficient indicates an increase in the mass of the target protein. In certain embodiments, the change in the diffusion coefficient of the target protein comprising the disordered domain compared to the reference target protein diffusion coefficient indicates a decrease in the mass of the target protein. In certain embodiments, the change in the diffusion coefficient of the target protein comprising the disordered domain compared to the reference target protein diffusion coefficient indicates an oligomerization process. In certain embodiments, a plurality of target proteins are conjugated to a plurality of fluorescent labels. In certain embodiments, the interaction between the target protein and the test molecule is reversible. In certain embodiments, the interaction between the target protein and the test molecule is irreversible.

In certain embodiments directed to identifying an interaction between a test molecule and a target protein comprising a disordered domain, the target protein comprising the disordered domain is selected from the group consisting of an antibody, a receptor, a structural protein, and an enzyme. In certain embodiments, the target protein comprising the disordered domain does not comprise a structured domain. In certain embodiments, the target protein comprising the disordered domain is a fragment of a native full-length protein. In certain embodiments, the target protein comprising the disordered domain is labeled. In certain embodiments, the target protein comprising the disordered domain is fluorescently labeled. In certain embodiments, the test molecule is fluorescently labeled. In certain embodiments, the target protein comprising the disordered domain is purified.

In certain embodiments directed to identifying an interaction between a test molecule and a target protein comprising a disordered domain, the test molecule is an organic molecule that is less than about 1 kDa. In certain embodiments, the test molecule is selected from the group consisting of a peptide, a protein, a nucleic acid, a carbohydrate, and a lipid. In certain embodiments, the protein is selected from the group consisting of an antibody, a receptor, and an enzyme. In certain embodiments, the protein comprises a disordered domain. In certain embodiments, the protein does not comprise a structured domain. In certain embodiments, the peptide is a ligand. In certain embodiments, the test molecule is a nanomaterial. In certain embodiments, the test molecule is labeled. In certain embodiments, the test molecule is fluorescently labeled. In certain embodiments, the test molecule is labeled with a fluorescent protein. In certain embodiments, the test molecule is labeled by conjugation of a synthetic nanomaterial or polymer. In certain embodiments, the test molecule is purified. In certain embodiments, the test molecule is a component of a mixture. In certain embodiments, the test molecule is a component of a mixture composed of a bacterial extract, cell extract, tissue extract, plant extract, or animal extract. In certain embodiments, the bacterial extract, cell extract, tissue extract, plant extract, or animal extract is a lysate. In certain embodiments, the test molecule is a component of a mixture composed of serum, blood, and other biological samples. In certain embodiments, the target molecule is a component of a mixture comprising a buffer and glycerol.

In certain embodiments directed to identifying an interaction between a test molecule and a target protein comprising a disordered domain, the interaction between the target protein comprising the disordered domain and the test molecule results in a conformational change in the target molecule. In certain embodiments, the conformational change in the target molecule results in a change in the target protein's temperature stability. In certain embodiments, the interaction between the target protein comprising the disordered domain and the test molecule results in an increase in the mass of the target protein. In certain embodiments, the interaction between the target protein comprising the disordered domain and the test molecule results in a decrease in the mass of the target protein. In certain embodiments, the interaction between the target protein comprising the disordered domain and the test molecule results in oligomerization processes.

In another aspect, the present disclosure provides methods, e.g., high throughput methods, for analyzing a target molecule in a test solution. In certain embodiments, the methods comprise: (a) tracking a plurality of the target molecule over time in the test solution to provide a plurality of spatiotemporal trajectories and/or measurements of rotational motion; (b) analyzing the plurality of spatiotemporal trajectories and/or measurements of rotational motion to determine the target molecule's movement in the test solution; and (c) comparing the target molecule's movement obtained in (b) with a reference target molecule's movement, wherein the reference target molecule's movement is the movement of the target molecule in a reference solution; wherein: (i) a change in the target molecule's movement compared to the reference target molecule's movement indicates that the target molecule is affected by the test solution; or (ii) no change in the target molecule's movement compared to the reference target molecule's movement indicates that the target molecule is not affected by the test solution. In certain embodiments, the duration and/or reversibility of the target molecule's movement change compared to the duration and/or reversibility of the reference target molecule's movement change indicates that the target molecule is affected by the test solution.

In certain embodiments, the change in movement associated with analyzing a target molecule in a test solution is a change in (a) diffusion coefficient obtained from a maximum likelihood estimator; (b) geometric mean posterior diffusion coefficient; (c) the median of the jump length distribution; (d) 3rd quartile of the jump length distribution; (e) median radius of gyration; (f) mean posterior diffusion coefficient; (g) mean squared displacement; (h) median bond angle; (i) spatiotemporal trajectory length; or (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.

In certain embodiments directed to analyzing a target molecule in a test solution, the test solution comprises a chaotropic agent. In certain embodiments, the chaotropic agent is urea. In certain embodiments, the test solution comprises a viscosity agent. In certain embodiments, the viscosity agent is glycerol. In certain embodiments, the test solution comprises a gradient. In certain embodiments, the gradient is a temperature gradient, a chemical gradient, or a combination thereof. In certain embodiments, the test solution comprises at least two phases.

In certain embodiments directed to analyzing a target molecule in a test solution, the target molecule is an organic molecule less than 1 kDa. In certain embodiments, the target molecule is selected from the group consisting of a peptide, a protein, a nucleic acid, a carbohydrate, and a lipid. In certain embodiments, the protein is selected from the group consisting of an antibody, a receptor, and an enzyme. In certain embodiments, the protein comprises a disordered domain. In certain embodiments, the protein does not comprise a structured domain. In certain embodiments, the peptide is a ligand. In certain embodiments, the target molecule is a nanomaterial. In certain embodiments, the target molecule is a synthetic polymer. In certain embodiments, the target molecule is labeled. In certain embodiments, the target molecule is fluorescently labeled. In certain embodiments, the target molecule is labeled with a fluorescent protein. In certain embodiments, the target molecule is labeled by conjugation of a synthetic nanomaterial or polymer. In certain embodiments, the target molecule is purified. In certain embodiments, the interaction between the target protein and the test solution results in a conformational change in the target molecule.

In certain embodiments directed to analyzing a target molecule in a test solution, the sample used in connection with any of the methods described herein, comprises a solution. For example, but not by way of limitation, where an interaction is being determined between a target molecule, e.g., a target protein comprising a disordered domain, and a test molecule, a change in the interaction between the target molecule and the solution in the presence of the test molecule can be identified. In certain embodiments, however, no test molecule need be present, and the interaction of the target molecule and the solution can be determined via a change in the movement of the target molecule in various solutions. In certain embodiments, the solution comprises a pH buffering agent, a salt, a chaotropic agent, a crowding agent, a carrier, a viscosity agent, a detergent, a reducing reagent, or a combination thereof. In certain embodiments, the viscosity agent comprises glycerol, sucrose, polyethylene glycol, dextran, ficoll, polyvinyl alcohol, and/or polyvinylpyrrolidone. In certain embodiments, the solution comprises at least about 0.1%, 1%, 10%, 20%, 30%, 40%, 50%, 60%, 70%, 80%, 90% or 99% glycerol or ranges of glycerol bounded by said glycerol ranges. In certain embodiments, the chaotropic agents comprises urea, guanidinium, alcohols, or ketones etc. In certain embodiments, the crowding agent comprises one or more of a polyethylene glycol (PEG) polymer, Ficoll, a dextran polymer, or a protein (e.g., ovalbumin or hemoglobin). In certain embodiments, the carrier is a protein or protein mixture. For example, but not by way of limitation, a carrier protein or protein mixture prevents nonspecific interactions between the target molecule and/or test molecule with other molecules and surfaces. Certain non-limiting examples of carriers include: bovine serum albumin (BSA); lysozyme; casein; non-fat dry milk; and other biological extracts. In certain embodiments, the solution comprises a temperature gradient, a chemical gradient, at least two liquid phases, or a combination thereof. In certain embodiments, the sample has a volume of up to about 1 mL. In certain embodiments, the sample has a volume in the range of about 0.1 μL to about 100 μL. In certain embodiments, the sample has a volume of about 1 μL, about 5 μL, about 10 μL, about 20 μL, about 30 μL, about 40 μL, about 50 μL, about 60 μL, about 70 μL, about 80 μL, about 90 μL or about 100 μL.

In another aspect, the present disclosure provides methods, e.g., high throughput methods, for analyzing a test solution comprising a target molecule. In certain embodiments, the methods comprise: (a) tracking a plurality of a target molecule over time in the test solution to provide a plurality of spatiotemporal trajectories and/or measurements of rotational motion; (b) analyzing the plurality of spatiotemporal trajectories and/or measurements of rotational motion to determine the target molecule's movement in the test solution; and (c) comparing the target molecule's movement obtained in (b) with a reference target molecule's movement, wherein the reference target molecule's movement is the movement of the target molecule in a reference solution; wherein: the comparing of step (c) is used to determine a property of the test solution.

In certain embodiments directed to analyzing a test solution, the target molecule's movement is calculated as a (a) diffusion coefficient obtained from a maximum likelihood estimator; (b) geometric mean posterior diffusion coefficient; (c) the median of the jump length distribution; (d) 3rd quartile of the jump length distribution; (e) median radius of gyration; (f) mean posterior diffusion coefficient; (g) mean squared displacement; (h) median bond angle; (i) spatiotemporal trajectory length; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.

In certain embodiments directed to analyzing a test solution, the test solution comprises a chaotropic agent. In certain embodiments, the chaotropic agent is urea. In certain embodiments, the test solution comprises a viscosity agent. In certain embodiments, the viscosity agent is glycerol. In certain embodiments, the test solution comprises a gradient. In certain embodiments, the gradient is a temperature gradient, a chemical gradient, or a combination thereof. In certain embodiments, the test solution comprises at least two phases. In certain embodiments, the test solution is a biological sample taken from a subject.

In certain embodiments directed to analyzing a test solution, the target molecule is an organic molecule less than 1 kDa. In certain embodiments, the target molecule is selected from the group consisting of a peptide, a protein, a nucleic acid, a carbohydrate, and a lipid. In certain embodiments, the protein is selected from the group consisting of an antibody, a receptor, and an enzyme. In certain embodiments, the protein comprises a disordered domain. In certain embodiments, the protein does not comprise a structured domain. In certain embodiments, the peptide is a ligand. In certain embodiments, the target molecule is a nanomaterial. In certain embodiments, the target molecule is a synthetic polymer. In certain embodiments, the target molecule is labeled. In certain embodiments, the target molecule is fluorescently labeled. In certain embodiments, the target molecule is labeled with a fluorescent protein. In certain embodiments, the target molecule is labeled by conjugation of a synthetic nanomaterial or polymer. In certain embodiments, the target molecule is purified. In certain embodiments, the interaction between the target protein and the test solution results in a conformational change in the target molecule.

In certain embodiments directed to analyzing a test solution, the determined property of the test solution is pH, an ion concentration, an organic molecule concentration, or a viscoelastic property. In some embodiments, the viscoelastic property is viscosity. In certain embodiments, the property of the test solution is used to diagnose a disease in the subject. In certain embodiments, the disease is cancer.

In certain embodiments directed to analyzing a test solution, the sample used in connection with any of the methods described herein, comprises a solution. For example, but not by way of limitation, where an interaction is being determined between a target molecule, e.g., a target protein comprising a disordered domain, and a test molecule, a change in the interaction between the target molecule and the solution in the presence of the test molecule can be identified. In certain embodiments, however, no test molecule need be present, and the interaction of the target molecule and the solution can be determined via a change in the movement of the target molecule in various solutions. In certain embodiments, the solution comprises a pH buffering agent, a salt, a chaotropic agent, a crowding agent, a carrier, a viscosity agent, a detergent, a reducing reagent, or a combination thereof. In certain embodiments, the viscosity agent comprises glycerol, sucrose, polyethylene glycol, dextran, ficoll, polyvinyl alcohol, and/or polyvinylpyrrolidone. In certain embodiments, the solution comprises at least about 0.1%, 1%, 10%, 20%, 30%, 40%, 50%, 60%, 70%, 80%, 90% or 99% glycerol or ranges of glycerol bounded by said glycerol ranges. In certain embodiments, the chaotropic agents comprises urea, guanidinium, alcohols, or ketones etc. In certain embodiments, the crowding agent comprises one or more of a polyethylene glycol (PEG) polymer, Ficoll, a dextran polymer, or a protein (e.g., ovalbumin or hemoglobin). In certain embodiments, the carrier is a protein or protein mixture. For example, but not by way of limitation, a carrier protein or protein mixture prevents nonspecific interactions between the target molecule and/or test molecule with other molecules and surfaces. Non-limiting examples of carriers include: bovine serum albumin (BSA); lysozyme; casein; non-fat dry milk; and other biological extracts. In certain embodiments, the solution comprises a temperature gradient, a chemical gradient, at least two liquid phases, or a combination thereof. In certain embodiments, the sample has a volume of up to about 1 mL. In certain embodiments, the sample has a volume in the range of about 0.1 μL to about 100 μL. In certain embodiments, the sample has a volume of about 1 μL, about 5 μL, about 10 μL, about 20 μL, about 30 μL, about 40 μL, about 50 μL, about 60 μL, about 70 μL, about 80 μL, about 90 μL or about 100 μL.

In certain embodiments, the change in movement determined by the disclosed methods is calculated as a change in the polarization of conjugated fluorescent labels. In certain embodiments, the methods for determining the change in the polarization of conjugated fluorescent labels can comprise (a) Time-Correlated Single-Photon Counting (TCSPC), (b) step-scan pump-probe techniques, (c) ultra-long lifetime fluorophores, and/or (d) advanced optical techniques. In certain embodiments, the target molecule and/or test molecule is labeled with an ultra-long lifetime fluorophore.

In another aspect, the present disclosure further provides systems for performing the disclosed methods.

### BRIEF DESCRIPTION OF THE DRAWINGS

The patent or application file includes at least one drawing executed in color. Copies of this patent or patent application publication with color drawing(s) will be provided by the Office upon request and payment of the necessary fee.

FIGS. 1A-1C provide a comparison of the diffusion of two highly similar proteins, HaloTag and Halo-Protein 1 fusion using the presently disclosed methods. FIG. 1A provides illustrations of the Protein Data Bank (PBD) structures of the exemplary proteins. A tail of 51 amino acid residues is represented with coils in the HaloTag illustration on the left. FIG. 1B provides a table that summarizes the theoretical and measured diffusion coefficient of each protein (measured at 10 pM) under 10% or 30% glycerol as determined by an exemplary method of the present disclosure. FIG. 1C provides a graph that shows the measured diffusion coefficients of HaloTag and Halo-Protein 1 (mean−/+98% confidence interval) over the number of fields of view (FOVs) used for the calculation.

FIGS. 2A-2B provide an analysis of protein-protein interactions (PPIs) using SMT. FIG. 2A provides the diffusion coefficient of Halo-Protein 1 (40 pM) measured in 30% glycerol with different concentrations of Protein 2 as determined by an exemplary method of the present disclosure. FIG. 2B provides the diffusion coefficient Halo-Protein 1 (40 pM) measured in the presence of 30 nM Protein 2 and increasing concentrations of unlabeled Protein 1 peptide as a competitor as determined by an exemplary method of the present disclosure.

FIGS. 3A-3E provide examples of distinguishing conformational changes using SMT. FIG. 3A provides the diffusion coefficient of sortase-tag-labeled Protein 3 (closed conformation) and Protein 3 mutant (open conformation) in 30% glycerol as determined by an exemplary method of the present disclosure. FIG. 3B provides the diffusion coefficient of Protein 3 mutant in response to addition of two small molecule ligands (Molecule M1 and Molecule M2) that can convert it to the closed conformation as determined by an exemplary method of the present disclosure. FIG. 3C shows the state array profiles of Protein 3 and Protein 3 mutant with different concentrations of Molecule M1 or MX added. FIG. 3D provides quantifications of FIG. 3C top left and bottom right panels. FIG. 3E is the state array profile of Protein 3 in the presence of 16 μM of Molecule M1 and a dose titration of Molecule MX, and its quantification.

FIGS. 4A-4F provide examples of buffer changes for analyzing changes elicited by ligand binding to a relatively rigid protein structure. FIG. 4A provides an exemplary structure showing the overlay without ligand and with a ligand (“Molecule M3”) bound form of Protein 4. FIG. 4B provides the diffusion coefficient of the sortase-tag labeled Protein 4 in 30% glycerol in the presence of DMSO (vector control) without ligand or with 3 μM Molecule M3 as determined by an exemplary method of the present disclosure. FIG. 4C shows the changes in the diffusion coefficient of Protein 4 in the presence of different chaotropic agents (urea, guanidinium, acetonitrile and acetone) with DMSO (without ligand) or in the presence of Molecule M3 ligand (3 μM) as determined by an exemplary method of the present disclosure. FIG. 4D shows the changes in the diffusion coefficient of Protein 4 in the presence of different chaotropic agents (acetone, acetonitrile, urea, guanidinium and DMSO) with or without Molecule M3 (3 μM) as determined by an exemplary method of the present disclosure. Here high concentrations of DMSO were used as a chaotropic agent, which differs from the small amount used elsewhere herein as vector controls for chemical treatment. FIG. 4E shows the changes in the diffusion coefficient of Protein 4 in the presence of different concentrations of chaotropic agents (urea, guanidinium, and acetone) in DMSO (without ligand) or in the presence of ligand Molecule M3 (3 μM) as determined by an exemplary method of the present disclosure. Data are presented as box plots. FIG. 4F shows a dose titration of Molecule M3 and its analog compounds (M3 analog 1 through M3 analog 6) to Protein 4 in the presence of 6 M urea. The table provides the measured Tm change in differential scanning fluorimetry (DSF) assay and IC₅₀ determined by fluorescence polarization (FP) assay. The apparent EC₅₀ determined by SMT was plotted against DSF (bottom left) and FP (bottom right) potency. EC₅₀ and IC₅₀ of Protein 4 Tool Compound 2 were estimated based on data available.

FIG. 5 provides the diffusion coefficient of an intrinsically disordered region (IDR), found within Protein 5, and its response to the addition of a covalent binder Molecule M4 as determined by an exemplary method of the present disclosure.

FIGS. 6A-6D highlight that an exemplary SMT method of the present disclosure enables in solution measurement of protein diffusion. FIG. 6A provides representative sequential images of SMT (top) with overlaid tracks (bottom) for JF₅₄₉-labeled free HaloTag protein at 30% glycerol for a 192×192 pixels ROI at 200 Hz. Scale bar represents 2 μm. FIG. 6B provides the mean diffusion coefficient as a function of medium viscosity compared to theoretical Stokes-Einstein equation. FIG. 6C provides the Halo-Protein 1 diffusion measured with a dose response of either monomeric (monovalent/MV) versus dimeric (bivalent/BV) Protein 2 (n=8 wells). FIG. 6D provides the diffusion coefficient of the Protein 1/Protein 2 complex measured across a dose response of unlabeled Protein 1 and Molecule B1 (n=32 FOVs from two distinct plates). Error bars denote S.D.

### DETAILED DESCRIPTION

The presently disclosed subject matter relates to methods, e.g., high throughput methods, for analyzing single molecules in cell-free systems. In certain embodiments, the methods described herein can be used for a variety of biophysical analysis applications including, but not limited to, drug discovery activities, such as compound library screening and the elucidation of structure-activity relationships (SAR). In certain embodiments, the methods described herein can be used to characterize both known and novel pathway contributions to larger molecular assemblies comprising the target, such as protein signaling interaction networks. The presently disclosed subject matter further provides system for performing such methods.

The subject matter of the present disclosure is described with reference to the figures. The figures are not drawn to scale and they are provided merely to illustrate aspects disclosed herein. It should be understood that numerous specific details, relationships and methods are set forth to provide a more complete understanding of the subject matter disclosed herein. For purposes of clarity of disclosure and not by way of limitation, the detailed description is divided into the following subsections:
1. Definitions;
2. Molecules;
3. SMT Methods for Biophysical Analysis;
4. SMT Systems for Biophysical Analysis;
5. Examples; and
6. Exemplary Embodiments

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the presently disclosed subject matter. All publications, patent applications, patents and other references mentioned herein are incorporated by reference in their entirety. The materials, methods, and examples disclosed herein are illustrative only and not intended to be limiting.

As used herein, the term “cell-free system” refers to a system that allows for the biophysical analysis of a molecule outside the context of an intact whole cell. Samples comprising such cell-free systems are not precluded from comprising intact whole cells or cell debris. Instead, the analytical focus of a cell-free system is external to intact whole cells. For example, but not by way of limitation, a cell-free system can comprise a cellular extract that nevertheless comprises intact whole cells or cell fragments, but where the analytical focus of the cell-free system occurs within the cellular extract and not within the intact whole cells. In certain embodiments, cell-free systems do not comprise cellular extracts and instead comprise a solution into which the target molecule and/or test molecules described herein can be disposed. In certain embodiments, the cellular extract is a lysate, e.g., a bacterial cell extract, a fungal cell extract, an animal tissue or cell extract, or a plant tissue or cell extract.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other instances “comprising,” “consisting of”, and “consisting essentially of,” the instances or elements presented herein, whether explicitly set forth or not.

For the recitation of numeric ranges herein, each intervening number within the range is explicitly contemplated with the same degree of precision. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “antibody” herein is used in the broadest sense and encompasses various antibody structures, including but not limited to monoclonal antibodies, polyclonal antibodies, multispecific antibodies and antibody fragments so long as they exhibit the desired antigen-binding activity. Non-limiting examples of antibody fragments include Fv, Fab, Fab′, Fab′-SH, F(ab′)₂, diabodies, linear antibodies, single-chain antibody molecules (e.g., scFv) and multispecific antibodies formed from antibody fragments.

The term “chaotropic agent,” as used herein, refers to a substance that disrupts the structure of, and/or denatures, macromolecules such as proteins and nucleic acids (e.g., DNA and RNA). In certain embodiments, chaotropic agents interfere with intermolecular and/or intramolecular interactions mediated by non-covalent forces such as hydrogen bonds, van der Waals forces and hydrophobic effects.

As used herein, the term “contacting” a sample, e.g., a cell-free sample, refers to exposing a sample to a molecule, e.g., a test molecule. The contacting can be accomplished using any suitable methods. For example, but not by way of limitation, “contacting” can be accomplished by adding the molecule, e.g., a test molecule, to the sample, e.g., a vessel holding the sample.

As used herein, the term “fluorescent molecule” refers to any molecule that emits a fluorescent signal. In certain embodiments, the fluorescent emission occurs in response to exposure to light of a particular wavelength. An example of a naturally occurring fluorescent protein is Green Fluorescent Protein (GFP). In certain embodiments, however, a test molecule or a target molecule can be adapted to emit a fluorescent signal via the introduction of an encoded fluorescent tag, e.g., a protein sequence is fused to a target protein to render it fluorescent. In certain embodiments, a test molecule or a target molecule can be adapted to emit a fluorescent signal through binding of a fluorescent ligand. Non-limiting examples of such encoded fluorescent tags include: Halo tags, SNAP tags, CLIP tags, TMP tags, and SunTags. Additionally, or alternatively, a test molecule or a target molecule can be adapted to emit a fluorescent signal via coupling to a fluorescent dye molecule, e.g., amine- or sulfhydryl-reactive dyes. Additionally, or alternatively, a test molecule or a target molecule can be adapted to emit a fluorescent signal via coupling to a fluorescent artificial material, e.g., polystyrene beads, Quantum dots, and/or Nanodiamonds.

As defined herein, “movement” of a molecule refers to a temporal change in the position, orientation, conformation and/or intermolecular binding of a molecule.

In certain embodiments, a molecule's movement can be quantified by analysis of changes in spatial coordinates in sequential timepoints, e.g., the analysis of spatiotemporal trajectories. Movement characterized in this way can also include, but not be limited to, measurements of diffusion coefficients. For example, but not by way of limitation, the measurement can be of a diffusion coefficient maximum likelihood estimator, defined as an estimate of the maximum likelihood diffusion coefficient for the plurality of spatiotemporal trajectories under a single-state diffusion model with constant localization error. In certain embodiments, molecule movement may be measured through analysis of the product of the link-generating algorithm. Movement characterized in this way can include, but not be limited to, the mean posterior diffusion coefficient, or the mean of the posterior probability distribution of coefficients from a probabilistic linking algorithm. Movement characterized in this way can include, but not be limited to, the geometric mean posterior diffusion coefficient, or the mean of the log-scaled posterior probability distribution of coefficients from a probabilistic linking algorithm. Movement characterized in this way can include, but not be limited to, measurements of the jump length distribution. For example, for a given a set of protein displacements between one timepoint and a subsequent timepoint, a histogram can be constructed of the probability of each of the displacement lengths (“jump lengths”). Quantiles of this distribution can be used to describe the motion of the molecule. In certain embodiments the quantile used is the median of the jump length distribution. In certain embodiments, the quantile used is the 3rd quartile of the jump length distribution. Movement characterized in this way can include, but not be limited to, measurements of the mean squared displacement as defined by the average of the square of all displacements in a spatiotemporal trajectory, averaged over the plurality of spatiotemporal trajectories. Movement characterized in this way can also include, but not be limited to, measurements of the spatiotemporal trajectory length or distribution of spatiotemporal trajectory lengths. Movement characterized in this way can also include, but not be limited to, measurements of the mean radius of gyration, as defined by the root mean square distance of all coordinates in a spatiotemporal trajectory from the center of mass of the set of points contained in the spatiotemporal trajectory, averaged over the plurality of spatiotemporal trajectories. Movement characterized in this way can also include, but not be limited to, measurements of the mean bond angle, defined by the angle formed from three sequential spatial coordinates averaged over the plurality of spatiotemporal trajectories. In certain embodiments, molecule movement can be measured through model-dependent analysis of the plurality of spatiotemporal trajectories. Movement characterized in this way can include, but not be limited to, the fraction of immobile molecules (“f_{bound}”) as defined by two-state model fitting.

As used herein, the term “movement” encompasses changes in the direction as well as changes, both increases and decreases, in the speed and duration at which a target is traveling. Accordingly, tracking movement can, in certain embodiments, include determining that the target is not moving, e.g., when the target either is or is essentially in a static bound state. As noted above, such movement can be characterized in a variety of ways, including, but not limited to, quantifying (a) a diffusion coefficient of the plurality of spatiotemporal trajectories obtained from a maximum likelihood estimator; (b) geometric mean posterior diffusion coefficient of the plurality of spatiotemporal trajectories; (c) the median of the jump length distribution of the plurality of spatiotemporal trajectories; (b) 3rd quartile of the jump length distribution of the plurality of spatiotemporal trajectories; (c) median radius of gyration of the plurality of spatiotemporal trajectories; (d) mean posterior diffusion coefficient of the plurality of spatiotemporal trajectories; (f) mean squared displacement of the plurality of spatiotemporal trajectories; (g) median bond angle of the plurality of spatiotemporal trajectories; (i) the spatiotemporal trajectory length of the plurality of spatiotemporal trajectories; (j) anisotropy decay time; (k) state occupation via inference; (l) spatial extent of detection; (m) number and wavelength of conjugated fluorescent labels; (n) occupancy in different diffusive states obtained from state arrays; and/or (o) polarization of conjugated fluorescent labels.

As used herein the term “spatiotemporal trajectory” refers to the set of spatial coordinates corresponding to the position of an observation of a molecule linked in time. In certain embodiments, a plurality of spatiotemporal trajectories can be constructed algorithmically by linking a plurality of molecules whose positions have been determined in successive time points. In certain embodiments, a plurality of spatiotemporal trajectories can be constructed conservatively by linking only spots within a fixed search radius when no other links are plausible. In certain embodiments, a plurality of spatiotemporal trajectories can be constructed probabilistically.

In certain embodiments, a molecule's movement can be quantified by analysis of changes in the rotational motion of the molecule at sequential timepoints. For example, but not by way limitation, movement characterized in this way can be determined as a measurement of fluorescence polarization. Fluorescence polarization is a measurement of changes in the dipole moment orientation of a fluorescent molecule (e.g., coupled to a target molecule or test molecule) over the time period between absorption and emission events. For example, but not by way of limitation, if a fluorophore is excited with polarized light, a slowly rotating molecule comprising that fluorophore will emit more light retaining the original polarization than a faster rotating molecule. Thus, by measuring the fluorescence polarization, it is possible to determine a target molecule's or a test molecule's rotational motion and make use of that information to calculate measurements of rotational motion.

In certain embodiments, the measurements of rotational motion described herein will be calculated as a function of anisotropy decay time. The systems described herein allow for detection of anisotropy decay time and which can be calculated using strategies known in the art, e.g., those outlined in “Time-Dependent Anisotropy Decays” Lakowicz, J. R. (eds) Principles of Fluorescence Spectroscopy. Springer, Boston, MA. https://doi.org/10.1007/978-0-387-46312-4_11), which is hereby incorporated by reference in its entirety. In certain embodiments, the measurements of rotational motion described herein will be calculated as a function of the intensity change of fluorescence polarization. The systems described herein allow for detection of intensity changes of fluorescence polarization, which can then be calculated using strategies known in the art. In certain embodiments, the measurements of rotational motion described herein will be calculated as a function of the relaxation time of rotational diffusion. The systems described herein allow for detection of intensity changes of relaxation time of rotational diffusion, which can then be calculated using strategies known in the art.

In certain embodiments, a target molecule's movement can be quantified by analysis of changes in the conformation of the target molecule in sequential timepoints. In certain embodiments, movement characterized in this way can include, but not be limited to, measurements of changes in the secondary, tertiary, and quaternary structure of a target protein, e.g. folding and unfolding processes and/or restructuring of the target protein's internal conformation.

In certain embodiments, a molecule's movement can be quantified by analysis of changes in the intermolecular binding of the target molecule in sequential timepoints. In certain embodiments, movement characterized in this way can include, but not be limited to, measurements of homogenous or heterogenous binding events, e.g., oligomerization processes.

As used herein, the movement being detected, including, but not limited to, any change in movement, can occur in response to any environmental or other factor (e.g., presence of a test molecule). For example, but not by way of limitation, the movement, or lack thereof, can be elicited by: (A) molecule addition; (B) a change in temperature; (C) a change in oxygen concentration, e.g., introduction of a hypoxic condition; (D) mechanical stress; (E) a change in pH; (F) a change in light exposure (e.g., increasing or decreasing intensity); (G) a change in solution composition, e.g. changes in viscosity and flow; and/or (H) a change in electric or ionic stimulation.

As used herein, the term “plurality” refers to a number larger than one. In certain embodiments, the term “plurality of target molecules” refers to a number of target molecules larger than one. For example, but not by way of limitation, a “plurality of target molecules” can include at least about 10, at least about 50, at least about 100, at least about 200, at least about 300, at least about 400, at least about 500, at least about 600, at least about 700, at least about 800, at least about 900, at least about 1000, at least about 5,000, at least about 10,000, at least about 50,000, at least about 100,000, at least about 500,000 or at least about 1,000,000 target molecules. In certain embodiments, the term “plurality of test molecules” refers to a number of test molecules larger than one. For example, but not by way of limitation, a plurality of test molecules can include at least about 10, at least about 50, at least about 100, at least about 200, at least about 300, at least about 400, at least about 500, at least about 600, at least about 700, at least about 800, at least about 900, at least about 1000, at least about 5,000, at least about 10,000, at least about 50,000, at least about 100,000, at least about 500,000 or at least about 1,000,000 test molecules.

As used herein “intrinsically disordered proteins” (IDPs) or “intrinsically disordered regions” (IDRs) refer to proteins and peptide fragments of various lengths that do not exist as stably folded structures that can be defined by classical structural biology tools such as X-ray crystallography and electron microscopy under physiological conditions. See, e.g., Uversky and Dunker 2010 Biochimica et Biophysica Acta (1804:1231-1264) (doi:10.1016/j.bbapap.2010.01.017) and van der Lee et al., 2014 Chemical Reviews (114:6589-6631) (doi:10.1021/cr400525m)

As used herein, the term “high throughput” in connection with the compositions, methods, and systems described herein refers to the ability to rapidly process a plurality of samples. For example, but not by way of limitation, the high throughput compositions, methods, and systems described herein can be configured to utilize a variety of sample processing plates, including 6-well plates, 12-well plates, 24-well plates, 48-well plates, 96-well plates, 384-well plates, 1536-well plates, or other multiwell plates capable of holding any number of separated samples. Moreover, the high throughput compositions, methods, and systems of the present disclosure can, in certain embodiments, can be configured to take advantage of robotic sample and plate processing as well as other computer-controlled sample and plate processing strategies. In certain embodiments, high throughput systems include, but not by way of limitation, microfluidic flow chambers. In certain embodiments, high throughput methods include the use of, but not by way of limitation, microfluidic flow chambers.

### 2. MOLECULES

The present disclosure provides high throughput methods for analyzing characteristics of single target molecules in cell-free systems. In certain embodiments, the present disclosure provides high throughput methods for analyzing characteristics of single target molecules in the presence of test molecules in cell-free systems. In certain embodiments, the target molecule and/or the test molecule to be analyzed by the present disclosure can be any chemically-defined entity.

In certain embodiments, the target molecule and/or test molecule can be an organic molecule less than about 10 kDa, less than about 5 kDa, less than about 1 kDa, less than about 250 Da, or less than about 100 Da. In certain embodiments, the target molecule and/or test molecule can be an inhibitor or agonist. For example, but not by way of limitation, the target molecule and/or test molecule can be a kinase inhibitor, e.g., a tyrosine kinase inhibitor.

In certain embodiments, the target molecule and/or test molecule is a protein. In certain embodiments, the target molecule and/or test molecule is a protein comprising a naturally occurring, e.g., wild type, amino acid sequence or fragment thereof. In certain embodiments, the target molecule and/or test molecule is a fusion of two or more proteins or fragments thereof. In certain embodiments, the target molecule and/or test molecule is a protein comprising one or more artificial, i.e., non-naturally occurring, amino acid sequences. In certain embodiments, the target molecule and/or test molecule is a protein that comprises natural and/or unnatural modifications, e.g., lipid protein conjugates, glycoproteins, and artificially modified proteins such as dye conjugated proteins and drug conjugated proteins, including antibody drug conjugates. In certain embodiments, a protein that can be analyzed by the methods of the present disclosure can include at least about 20, at least about 30, at least about 40, at least about 50, at least about 60, at least about 70, at least about 80, at least about 90, at least about 100, at least about 200, at least about 300, at least about 400, at least about 500, at least about 1,000, at least about 5,000, at least about 10,000, at least about 15,000, at least about 20,000 or at least about 30,000 amino acids. In certain embodiments, proteins that can be analyzed using the high throughput methods of the present disclosure include any protein including, but not limited to, proteins that contain one or more disulfide bonds, including multi-chain polypeptides comprising one or more inter- and/or intrachain disulfide bonds, as well as multi-chain polypeptides that do not comprise intrachain disulfide bonds.

In certain embodiments, a target protein and/or test protein of the present disclosure can be a signaling protein, e.g., a protein hormone, a cytokine, a kinase, a phosphatase, a receptor or other enzyme or transcription factor. In certain embodiments, a target protein and/or test protein of the present disclosure is a receptor. In certain embodiments, a target protein and/or test protein of the present disclosure is an enzyme. In certain embodiments, a target protein and/or test protein of the present disclosure is a contractile protein. In certain embodiments, a target protein and/or test protein of the present disclosure is a structural protein. In certain embodiments, a target protein and/or test protein of the present disclosure is a storage protein. In certain embodiments, a target protein and/or test protein of the present disclosure is a transport protein. In certain embodiments, a target protein and/or test protein of the present disclosure is an ion channel. In certain embodiments, a target protein and/or test protein of the present disclosure is an antibody or an antibody fragment.

In certain embodiments, a target protein and/or test protein can include one or more disordered domains. For example, but not by way of limitation, a target protein and/or test protein includes at least one polypeptide segment that lacks a stable three-dimensional structure. In certain embodiments, the target protein and/or test protein can include one or more structure domains and one or more disordered domains. In certain embodiments, the target protein and/or test protein does not include a structured domain. Non-limiting examples of proteins that include disordered domains comprise the Glucocorticoid receptor, Growth hormone receptor, Cellular tumor antigen p53, T-cell surface glycoprotein CD4, SHC-transforming protein 1, Estrogen receptor, Vitamin D3 receptor, Cyclin-H, Serine/threonine-protein kinase pim-1, Aryl hydrocarbon receptor, Androgen receptor, Cryptochrome-2, T-cell surface glycoprotein CD3 delta chain, Sulfotransferase 2B1, DNA repair protein RAD52 homolog, Huntingtin-interacting protein K and Ras-related protein Ral-A. Additional non-limiting examples of proteins that include disordered domains are disclosed in data bases and references listed in Uversky and Dunker 2010 Biochimica et Biophysica Acta (1804: 1231-1264) (doi: 10.1016/j.bbapap.2010.01.017) and van der Lee et al., 2014 Chemical Reviews (114:6589-6631) (doi: 10.1021/cr400525m), the entire contents of which are disclosed herein by reference in their entirety.

In certain embodiments, the target molecule and/or test molecule is a peptide. For example, but not by way of limitation, a peptide that can be analyzed by the methods of the present disclosure can include at about 2, about 5, about 10, about 15, about 20, about 25, about 30, about 35 or about 40 amino acids. In certain embodiments, the target molecule and/or test molecule is a peptide containing unnatural amino acids or other modifications.

In certain embodiments, the target molecule and/or test molecule is a nucleic acid. In certain embodiments, the nucleic acid can have a defined sequence. In certain embodiments, the nucleic acid comprises (A) ribonucleic acid (RNA), including, for example, modified RNA; (B) deoxyribonucleic acid (DNA), including, for example, modified DNA; as well as (C) combinations of (A) and (B). In certain embodiments, the nucleic acid comprises one or more categories of artificial nucleic acids including, for example, morpholino, locked nucleic acid (LNA), peptide nucleic acid (PNA), and xeno nucleic acid (XNA). In certain embodiments, the nucleic acid will be a single-stranded or double-stranded small interfering ribonucleic acid (e.g., a double-stranded siRNA), an antisense oligonucleotide, a ribozyme, a microRNA, or an aptamer. In certain embodiments, the nucleic acid molecule can be linear or circular. In certain embodiments, the nucleic acid can contain naturally occurring or non-naturally occurring nucleotides. Non-limiting examples of non-naturally occurring nucleotides include modified nucleotide bases with derivatized sugars or phosphate backbone linkages or chemically modified residues.

In certain embodiments, the target molecule and/or test molecule is a carbohydrate. In certain embodiments, the carbohydrate can be a monosaccharide (e.g., glucose, fructose, xylose, ribose and galactose), a disaccharide (e.g., sucrose) or an oligo- or polysaccharide (e.g., cellulose and starch).

In certain embodiments, the target molecule and/or test molecule is a lipid. For example, but not by way of limitation, the lipid can be a triglyceride, a phospholipid and/or a sterol.

In certain embodiments, the target molecule and/or test molecule is a component of a mixture comprising a bacterial extract, cell extract, tissue extract, plant extract, or animal extract. In certain embodiments, the bacterial extract, cell extract, tissue extract, plant extract, or animal extract is a lysate. In certain embodiments, the target molecule and/or test molecule is a component of a mixture comprising serum, blood, and other biological samples. In certain embodiments, the target molecule and/or test molecule is a component of a mixture comprising one or more buffers and/or glycerol.

In certain embodiments, the target molecule can be a wild type form of the target molecule, e.g., the target molecule can be a protein that is a wild type form of the protein.

In certain embodiments, the target molecule can be a modified form of the target molecule. For example, but not by way of limitation, the modified form of a target molecule can be a mutated form of the target molecule. For example, but not by way of limitation, the target molecule can be a protein that is a mutant form of the protein. In certain embodiments, the mutant form of the protein includes at least one amino acid substitution (e.g., at least two, at least three, at least four, at least five, at least six, at least seven, at least eight, at least nine or at least ten or more amino acid substitutions) compared to the wild type form of the protein. In certain embodiments, the modified target molecule can be a post-translationally modified form of a target protein. In certain embodiments, the post-translational modifications include, but are not limited to, glycosylation, acetylation, phosphorylation, methylation, glycosylation and lipidation. Additional non-limiting examples of post-translational modifications are disclosed in Ramazi and Zahiri, Database (Oxford):baab012 (2021), the entire contents of which are disclosed herein by reference in its entirety. In certain embodiments, the modified target molecule can be a protein that contains unnatural amino acid(s). In certain embodiments, the modified target molecule can be a protein that contains unnatural amino acid(s) that are artificially modified. In certain embodiments, the modified target molecule can comprise conjugates, e.g., protein conjugates such as fusion proteins and proteins operably linked to non-protein compositions.

In certain embodiments, the target molecule is a homolog of a target molecule. In certain embodiments, the target molecule is an ortholog of a target molecule. In certain embodiments, the target molecule is a paralog of a target molecule.

In certain embodiments, the target molecule and/or test molecule can be present in a mixture of molecules, e.g., in a mixture of defined composition. For example, but not by way of limitation, the target molecule can be present in a mixture (e.g., sample) that includes one or more different target molecules, e.g., one or more target proteins, one or more target peptides, one or more target organic molecules of less than 1 kDa, one or more target carbohydrates, one or more target lipids, one or more target nucleic acids, one or more target proteins, one or more target peptides, one or more target organic molecules less than 1 kDa, one or more target carbohydrates, one or more target lipids and/or one or more target nucleic acids. In certain embodiments, a sample or mixture can include a first target molecule and a second target molecule. In certain embodiments, the first and second target molecules are different species of the target molecule. In certain embodiments, the first target molecule and the second target molecule are the same or different conformational states of the target molecule. In certain embodiments, distinguishing between the first target molecule and the second target molecule is achieved by a difference in their labels, e.g., fluorescent labels. In certain embodiments, more than two target molecules are analyzed, e.g., three or more target molecules are analyzed. In certain embodiments, the target molecule can be labeled. For example, but not by way of limitation, the target molecule can be fluorescently labeled, e.g., to be detected using an exemplary method of the present disclosure. In certain embodiments, the target molecule can be labeled via the addition of a fluorescent protein or a tag that can facilitate fluorescent labeling. Non-limiting examples of such fluorescent proteins include: Green Fluorescent Protein (GFP), Venus, monomeric Infrared Fluorescent Protein (mIFP), Long Stokes Shift monomeric Orange (LssmOrange), Tag Red Fluorescent Protein 657 (TagRFP657), monomeric Orange2 (mOrange2), monomeric Apple(mApple), Sapphire, monomeric Tag Blue Fluorescent Protein (mTagBFP2), tdTomato, monomeric Cherry (mCherry), Enhanced Yellow Fluorescent Protein (EYFP), monomeric Cerulean3 (mCerulean3), and Enhanced Green Fluorescent Protein (EGFP). Nonlimiting examples of tags that can facilitate fluorescent labeling include: Halo tag, SNAP tag, CLIP tag, TMP tag, and SunTag. In certain embodiments, the target molecule can be labeled to a fluorescent artificial material, e.g., polystyrene beads, Quantum dots and/or Nanodiamonds, to emit a fluorescent signal. In certain embodiments, the target molecule can be labeled via a common tags used for recombinant protein expression, nonlimiting examples including His-, GST-, FLAG-, HA-, Avi-tag. In certain embodiments, the target molecule can be biotinylated and labeled via avidin and derivatives. In certain embodiments, the target molecule can be labeled via a ligand, nonlimiting examples including natural or unnatural ligand that can bind to the protein or tag. In certain embodiments, the target molecule can be labeled via an antibody. In certain embodiments, the target molecule can be labeled via chemical reactions, nonlimiting examples including NHS-, Malemide-mediated conjugations. In certain embodiments, the target molecule can be labeled via chemical reactions specific for incorporated unnatural amino acids. In certain embodiments, the target molecules can be labeled via nonspecific absorption. In certain embodiments, the target molecules can be labeled by conjugation of a synthetic nanomaterial or polymer.

Alternatively or additionally, the test molecule can be labeled. For example, but not by way of limitation, the test molecule can be fluorescently labeled, e.g., to be detected using an exemplary method of the present disclosure. In certain embodiments, the test molecule can be labeled via a fluorescent protein. Nonlimiting examples of such fluorescent proteins include: Green Fluorescent Protein (GFP), Venus, monomeric Infrared Fluorescent Protein (mIFP), Long Stokes Shift monomeric Orange (LssmOrange), Tag Red Fluorescent Protein 657 (TagRFP657), monomeric Orange2 (mOrange2), monomeric Apple(mApple), Sapphire, monomeric Tag Blue Fluorescent Protein (mTagBFP2), tdTomato, monomeric Cherry (mCherry), Enhanced Yellow Fluorescent Protein (EYFP), monomeric Cerulean3 (mCerulean3), and Enhanced Green Fluorescent Protein (EGFP). Nonlimiting examples of tags that can facilitate fluorescent labeling include: Halo tag, SNAP tag, CLIP tag, TMP tag, and SunTag. In certain embodiments, the test molecule can be labeled to a fluorescent artificial material, e.g., polystyrene beads, Quantum dots and/or Nanodiamonds, to emit a fluorescent signal. In certain embodiments, the test molecule can be labeled via a ligand, nonlimiting examples including natural or unnatural ligand. In certain embodiments, the test molecule can be labeled via an antibody. In certain embodiments, the test molecule can be labeled via chemical reactions, nonlimiting examples including NHS-, Maleimide-mediated conjugations. In certain embodiments, the test molecules can be labeled via nonspecific absorption. In certain embodiments, the test molecules can be labeled by conjugation of a synthetic nanomaterial or polymer.

In certain embodiments, the target molecule and/or test molecule can be purified. For example, but not by way of limitation, the target molecule and/or test molecule can be purified from at least some of the components with which it was associate either when initially produced or generated. In certain embodiments, a purified target molecule and/or purified test molecule is at least about 10%, at least about 20%, at least about 30%, at least about 40%, at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99% pure. For example, but not by way of limitation, a target and/or test protein or peptide can be purified from the cell (and its components) in which it was expressed. In certain embodiments, the target molecule and/or test molecule can be partially purified. In certain embodiments, the target molecule and/or test molecule can be partially purified extract from biological samples. In certain embodiments, the target molecule and/or test molecule can be crude extract from biological samples. In certain embodiments, the target molecule and/or test molecule can be crude biological samples, nonlimiting examples including cell culture media, saliva, blood, serum, and other body fluids. In certain embodiments, the extract is a lysate (e.g., a cell or tissue lysate).

### 3. SMT METHODS FOR BIOPHYSICAL ANALYSIS

The present disclosure provides methods, e.g., high throughput methods, for analyzing single molecules in cell-free samples. In certain embodiments, the present disclosure provides methods for determining the movement of target molecules. In certain embodiments, the movement of target molecules is performed by tracking the target molecules over time to provide a plurality of spatiotemporal trajectories and/or measurements of rotational motion. Such movement can be in the presence or absence of other sample constituents, e.g., test molecules or specific sample solutions.

In certain embodiments, the movement of target molecules will be determined by analysis of a plurality of spatiotemporal trajectories and/or measurements of rotational motion detected by the method described herein. For example, but not by way of limitation, such analysis can address movement determined as: (a) a diffusion coefficient of the plurality of spatiotemporal trajectories obtained from a maximum likelihood estimator; (b) geometric mean posterior diffusion coefficient of the plurality of spatiotemporal trajectories; (c) the median of the jump length distribution of the plurality of spatiotemporal trajectories; (b) 3rd quartile of the jump length distribution of the plurality of spatiotemporal trajectories; (c) median radius of gyration of the plurality of spatiotemporal trajectories; (d) mean posterior diffusion coefficient of the plurality of spatiotemporal trajectories; (f) mean squared displacement of the plurality of spatiotemporal trajectories; (g) median bond angle of the plurality of spatiotemporal trajectories; (i) the spatiotemporal trajectory length of the plurality of spatiotemporal trajectories; (j) anisotropy decay time; (k) state occupation via inference; (l) spatial extent of detection; (m) number and wavelength of conjugated fluorescent labels; (n) occupancy in different diffusive states obtained from state arrays; and/or (o) polarization of conjugated fluorescent labels.

The sensitivity of the presently disclosed methods provides for the observation of a change in the movement of a target molecule compared to the movement of a reference target molecule. In certain embodiments, the movement of a reference target molecule can be the movement of a target molecule in the absence of a test molecule. In certain embodiments, the movement of a reference target molecule can be the movement of a target molecule in the presence of a reference solution. In certain embodiments, the movement of a reference target molecule can be the movement of a target molecule that is a different form of the target molecule being analyzed by the methods of the present disclosure. In certain embodiments, the movement of a reference target molecule can be the movement of a target molecule that is the same form of the target molecule being analyzed by the methods of the present disclosure.

In certain embodiments the movement of a target molecule is determined as the diffusion coefficient associated with the target molecule. In certain embodiments, the movement, e.g., diffusion coefficient, of target molecules determined by the methods, e.g., high throughput methods, of the present disclosure can be used to determine the identity of the target molecule and/or identify interactions between the target molecule and other molecules, e.g., test molecules or other solution components, in the cell-free sample.

In certain embodiments, the movement of a target molecule and the movement of a reference target molecule are determined as diffusion coefficients. In certain embodiments, the change in diffusion coefficient between the target molecule and the reference target molecule is at least about 0.001%. For example, but not by way of limitation, the change in the diffusion coefficient observed by the disclosed methods is at least about 0.001%, at least about 0.005%, at least about 0.01%, at least about 0.05%, at least about 0.1%, at least about 0.5%, at least about 1%, at least about 1.5%, at least about 2%, at least about 2.5%, at least about 3%, at least about 3.5%, at least about 4%, at least about 4.5%, at least about 5%, at least about 5.5%, at least about 6%, at least about 6.5%, at least about 7%, at least about 7.5%, at least about 8%, at least about 8.5%, at least about 9%, at least about 9.5% or at least about 10%. In certain embodiments, the change in the diffusion coefficient compared to the reference diffusion coefficient is at least about 0.001%. In certain embodiments, the change in the diffusion coefficient compared to the reference diffusion coefficient is at least about 0.005%. In certain embodiments, the change in the diffusion coefficient compared to the reference diffusion coefficient is at least about 0.01%. In certain embodiments, the change in the diffusion coefficient compared to the reference diffusion coefficient is at least about 0.05%. In certain embodiments, the change in the diffusion coefficient compared to the reference diffusion coefficient is at least about 0.1%. In certain embodiments, the change in the diffusion coefficient compared to the reference diffusion coefficient is at least about 0.5%. In certain embodiments, the change in the diffusion coefficient compared to a reference diffusion coefficient is at least about 1%. In certain embodiments, the change in the diffusion coefficient compared to the reference diffusion coefficient is at least about 2%. In certain embodiments, the change in the diffusion coefficient compared to the reference diffusion coefficient is at least about 3%. In certain embodiments, the change in the diffusion coefficient compared to the reference diffusion coefficient is at least about 4%. In certain embodiments, the change in the diffusion coefficient compared to the reference diffusion coefficient is at least about 5%. In certain embodiments, the change in the diffusion coefficient compared to the reference diffusion coefficient is at least about 6%. In certain embodiments, the change in the diffusion coefficient compared to the reference diffusion coefficient is at least about 7%. In certain embodiments, the change in the diffusion coefficient compared to the reference diffusion coefficient is at least about 8%. In certain embodiments, the change in the diffusion coefficient compared to the reference diffusion coefficient is at least about 9%. In certain embodiments, the change in the diffusion coefficient compared to the reference diffusion coefficient is at least about 10%.

In certain embodiments, the expected diffusion coefficient of a target molecule and the expected diffusion coefficient of a test molecule are estimated. In certain embodiments, diffusion coefficients of the target molecule and/or of the test molecule are measured with optimized tracking settings (e.g., search radius) for each frame rate. In certain embodiments, the measured diffusion coefficient of each molecule with the optimized tracking settings are used to fit estimated Stokes radius of each molecule in experimental conditions. In certain embodiments, the estimated Stokes radius is used to estimate the expected diffusion coefficient of the target molecule and of the test molecule. In certain embodiments, a comprehensive tracking model is used to estimate the expected diffusion coefficient of the target molecule and of the test molecule. In certain embodiments, the comprehensive tracking model accounts for jump truncation, tracking errors, and other effects. In certain embodiments, the comprehensive tracking model and the estimated Stokes radius is used to estimate the expected diffusion coefficient of the target molecule and of the test molecule.

### 3.1 Analysis of Forms of a Target Molecule

The present disclosure provides methods, e.g., high throughput methods, for analyzing the movement of a target molecule to determine the form of the target molecule. As used herein, “form” is not limited to the three dimensional conformation of a target molecule, but rather encompasses additional alternatives. For example, but not by way of limitation, a target molecule can take a variety of forms depending on the nature of the target molecule, e.g., a protein target molecule will have a wild type form, but can also have one or more modified forms, including, but not limited to, mutant forms, post-translational modified forms, alternatively spliced forms, or conjugated forms. For example, but not by way of limitation, methods of the present disclosure can allow for determining whether a target molecule is a post-translationally modified form of the target molecule (e.g., a post-translationally modified form of a target protein), whether a target molecule is an alternatively spliced form of the target molecule (e.g., an alternatively spliced form of a target protein), whether a target molecule is of a particular conformation (e.g., an open conformation of a target protein or a closed conformation of a target protein), a wild type form of the target molecule (e.g., a wild type form of a target protein), a mutant form of the target molecule (e.g., a mutant form of a target protein), a homolog of the target molecule (e.g., a homolog of the target protein), an ortholog of the target molecule (e.g., an ortholog of the target protein), a paralog of the target molecule (e.g., a paralog of the target protein), or an artificially modified form of the target molecule (e.g., an artificially modified from of the target protein).

In certain embodiments, methods, e.g., high throughput methods, for analyzing the movement of a target molecule to determine the form of the target molecule can include comparing the movement of the target molecule to the movement of a reference target molecule. In certain embodiments, the reference target molecule can be a different form of the target molecule being analyzed. In certain embodiments, the reference target molecule can be the same form of the target molecule being analyzed.

In certain embodiments, methods for determining the form of a target molecule can comprise: (a) tracking a plurality of the target molecule over time in a cell-free sample to provide a plurality of spatiotemporal trajectories and/or measurements of rotational motion; (b) analyzing the plurality of spatiotemporal trajectories and/or measurements of rotational motion to determine the target molecule's movement; and (c) comparing the target molecule's movement obtained in (b) with a reference target molecule's movement, wherein the reference target molecule's movement is the movement of one form of the target molecule; wherein: (i) a change in the target molecule's movement compared to the reference target molecule's movement indicates that the target molecule is of a different form than the reference target molecule; or (ii) no change in the target molecule's movement compared to the reference target molecule's movement indicates that the target molecule is of the same form of the target molecule. In certain embodiments, the duration and/or reversibility of the target molecule's movement change compared to the duration and/or reversibility of the reference target molecule's movement change indicates that the target molecule is of a different form than the reference target molecule. In certain embodiments, the duration of the target molecule's movement change compared to the duration of the reference target molecule's movement change indicates that the target molecule is of a different form than the reference target molecule. In certain embodiments, the reversibility of the target molecule's movement change compared to the reversibility of the reference target molecule's movement change indicates that the target molecule is of a different form than the reference target molecule.

In certain embodiments, the reference target molecule's movement that is compared to a target molecule's movement can correspond to the movement of a reference target molecule that is measured when the reference target molecule exists in a different form relative to the target molecule being analyzed by the methods of the present disclosure. For example, but not by way of limitation, the movement of the reference target molecule can be measured under conditions where the reference target molecule exists in a distinct conformation relative to the conformation of the target molecule to which its movement is being compared. In certain embodiments, the reference target molecule's movement corresponds to the movement of the reference target molecule when it is in the same form as the target molecule being analyzed by the methods of the present disclosure.

3.2 Analysis of Target Molecule Interactions with Test Solutions

The present disclosure further provides methods, e.g., high throughput methods, for analyzing the movement of a target molecule in a test solution. In certain embodiments, the test solution is a solution of known composition. In certain embodiments, the cell-free sample comprises the test solution. In certain embodiments, the test solution is a biological sample taken from a subject. In certain embodiments, the biological samples of different levels of purity comprise the test solution.

In certain embodiments, the test solution includes an agent for altering the conformation and/or oxidation or charge state of the target molecule. In certain embodiments, the test solution includes an agent for altering the conformation state of the target molecule, e.g., a target protein.

In certain embodiments, the test solution comprises one or more of: salts (e.g., sodium, magnesium, and calcium), buffers (e.g., acetate, citrate, bis-tris, carbonate, CAPS, TAPS, bicine, tris, tricine, TAPSO, HEPES, TES, MOPS, PIPES, cacodylate, SSC, MES, succinic acid, or phosphates), amino acids, acids, bases, surfactants, detergents (e.g., SDS, triton X-100, or Tween-20), chelators (e.g., ethylenediaminetetraacetic acid, phosphonates, or citric acid), preservatives, antibiotics, alcohols (e.g., methanol, ethanol, propanol, or isopropanol), reducing compounds, chaotropic agents, viscosity agents, oxidizing compounds, dyes, or biomolecules (e.g., ATP, GTP, NADPH, nucleic acids, proteins, enzymes (e.g., RNase or Proteinase K)).

In certain embodiments, the test solution includes an agent that reduces the structural stability of target molecules. In certain embodiments, the test solution includes a chaotropic agent. Non-limiting examples of chaotropic agents include n-butanol, ethanol, acetone, acetonitrile, guanidinium chloride, lithium perchlorate, lithium acetate, magnesium chloride, phenol, 2-propanol, sodium dodecyl sulfate, thiourea and urea. In certain embodiments, the chaotropic agent is guanidinium chloride. In certain embodiments, the chaotropic agent is urea. In certain embodiments, the chaotropic agent is acetone. In certain embodiments, the chaotropic agent is acetonitrile.

In certain embodiments, the test solution comprises an agent that includes the viscosity of the solution, e.g., a viscosity agent. In certain embodiments, the viscosity agent is glycerol. In certain embodiments, viscosity agent, e.g., glycerol, is present in the solution at a concentration of about 1% volume per volume (% v/v) or greater, about 2% v/v or greater, about 5% v/v or greater, about 10% v/v or greater, about 15% v/v or greater, about 20% v/v or greater, about 25% v/v or greater, about 30% v/v, about 35% v/v or greater, about 40% v/v or greater, about 45% v/v or greater, about 50% v/v or greater, about 55% v/v or greater, about 60% v/v or greater, about 65% v/v or greater, about 70% v/v or greater, about 75% v/v or greater, about 80% v/v or greater, about 85% v/v or greater, about 90% v/v or greater or about 95% v/v or greater.

In certain embodiments, the test solution comprises a gradient. In certain embodiments, the gradient is a temperature gradient, a chemical gradient, or a combination thereof. In certain embodiments, the gradient is a temperature gradient, e.g., the solution can include gradient of higher temperature to lower temperature. In certain embodiments, the temperature gradient is formed by the use of a laser. In certain embodiments, the gradient is a chemical gradient, e.g., the solution can include gradient of higher chemical concentration to lower chemical concentration.

In certain embodiments, the test solution includes at least two phases. In certain embodiments, the at least two phases comprise two liquid phases. In certain embodiments, the at least two phases comprise a liquid phase and a solid phase. For example, but not by way of limitation, the test solution can include two liquid phases comprising an aqueous phase and an oil phase. Alternatively, the test solution can include two or more aqueous phases. In certain embodiments, the test solution comprises a liquid phase and a solid phase where the liquid phase is an aqueous phase and the solid phase is a precipitate. In certain embodiments, the test solution comprises a liquid phase and a solid phase where the liquid phase is an aqueous phase and the solid phase is an aggregation. In certain embodiments, the test solution includes cells or other solid particles. In certain embodiments, the methods of the present disclosure allow for a determination of the movement of a target molecule at and/or across the interface between two phases of a solution.

In certain embodiments, the test solution is actively flowing through the interrogation area of the equipped device. As an example, but not limited to, control of the flow of the test solution can be realized by a microfluidic chip operated in flow-through. In certain embodiments, the flow is controlled with peristaltic or syringe pumps. In certain embodiments, the microfluidic chip contains one or more channels, one or more inlets, one or more outlets, and one or more interrogation areas. In certain embodiments, the microfluidic chip consists of, but not limited to, glass and/or polymers. In certain embodiments, the microfluidic flow profile is controlled by, but limited to, sheath flows.

In certain embodiments, the test solution is a homogenous solution. For example, but not by way of limitation, the test solution can have a uniform composition. In certain embodiments, the concentration of components, e.g., chemicals or agents as exemplified herein, is consistent throughout the solution. In certain embodiments, the concentration of a viscosity agent (e.g., glycerol) is consistent throughout the test solution.

In certain embodiments, the methods of the present disclosure comprise: (a) tracking a plurality of the target molecule over time in a test solution to provide a plurality of spatiotemporal trajectories and/or measurements of rotational motion; (b) analyzing the plurality of spatiotemporal trajectories and/or measurements of rotational motion to determine the target molecule's movement in the test solution; and (c) comparing the target molecule's movement obtained in (b) with a reference target molecule's movement, wherein the reference target molecule's movement is the movement of the target molecule in a reference solution; wherein: (i) a change in the target molecule's movement compared to the reference target molecule's movement indicates that the target molecule is affected by the test solution; or (ii) no change in the target molecule's movement compared to the reference target molecule's movement indicates that the target molecule is not affected by the test solution.

In certain embodiments, the comparing of step (c) further comprises comparing the duration and/or reversibility of the target molecule's movement change with the duration and/or reversibility of the reference target molecule's movement change. For example, but not by way of limitation, the duration of the target molecule's movement change compared to the duration of the reference target molecule's movement change indicates that the target molecule is affected by the test solution. In certain embodiments, the reversibility of the target molecule's movement change compared to the reversibility of the reference target molecule's movement change indicates that the target molecule is affected by the test solution.

In certain embodiments, the methods of the present disclosure comprise: (a) tracking a plurality of the target molecule over time in a test solution to provide a plurality of spatiotemporal trajectories and/or measurements of rotational motion; (b) analyzing the plurality of spatiotemporal trajectories and/or measurements of rotational motion to determine the target molecule's movement in the test solution; and (c) comparing the target molecule's movement obtained in (b) with a reference target molecule's movement, wherein the reference target molecule's movement is the movement of the target molecule in a reference solution; wherein: the comparing of step (c) is used to determine a property of the test solution. In certain embodiments, the comparing of step (c) further comprises comparing the duration and/or reversibility of the target molecule's movement change with the duration and/or reversibility of the reference target molecule's movement change.

In certain embodiments, the determined property of the test solution is pH, an ion concentration, an organic molecule concentration, or a viscoelastic property. In certain embodiments, the viscoelastic property is viscosity. In certain embodiments, the property of the test solution is used to diagnose a disease in the subject. In certain embodiments, the disease is cancer.

In certain embodiments, the reference solution can be a solution having the same composition as the test solution. In certain embodiments, the reference solution can be a solution having the same composition as the test solution except for one component, e.g., the viscosity agent or the chaotropic agent. In certain embodiments, the reference solution can be a solution that has a different composition as the test solution. In certain embodiments, the reference solution can be a second biological sample taken from the subject.

In certain embodiments, the interaction between the target protein and the test solution results in a conformational change in the target molecule. In certain embodiments, the interaction between the target protein and the test solution is reversible. In certain embodiments, the interaction between the target protein and the test solution is irreversible. In certain embodiments, the conformational change in the target molecule results in a change in the target molecule's temperature stability.

3.3 Analyzing Target Molecule Interactions with Test Molecules

Intermolecular interactions are essential aspects of the function of biological molecules. For example, but not by way of limitation, these crucial interactions include antibody-antigen, enzyme-coenzyme, enzyme-cofactor, enzyme-substrate, enzyme-allosteric/orthosteric regulator, ligand-receptor, ligand-coreceptor, protein-protein, protein-DNA, protein-RNA, protein-lipid, protein-cell surface, protein-viral particle, protein-nanoparticle interactions. The present disclosure provides methods, including high throughput methods, for identifying an interaction between a target molecule and a test molecule. In certain embodiments, the methods include detecting a direct or indirect interaction between the target molecule and the test molecule.

In certain embodiments, the methods, e.g., high throughput methods, for identifying an interaction between a target molecule and a test molecule comprises: (a) contacting a cell-free sample comprising a plurality of the target molecule with a plurality of the test molecule; (b) tracking a plurality of the target molecules over time to provide a plurality of spatiotemporal trajectories and/or measurements of rotational motion; (c) analyzing the plurality of spatiotemporal trajectories and/or measurements of rotational motion to determine the target molecule's movement in the presence of the test molecule; and (d) comparing the target molecule's movement obtained in (c) with a reference target molecule's movement, wherein the reference target molecule's movement is the movement of the target molecule in the absence of the test molecule; wherein a change in the target molecule's movement compared to the reference target molecule's movement indicates an interaction between the target molecule and the test molecule. In certain embodiments, the duration and/or reversibility of the target molecule's movement change compared to the duration and/or reversibility of the reference target molecule's movement change indicates an interaction between the target molecule and the test molecule. In certain embodiments, the interaction between the target molecule and the test molecule is reversible. In certain embodiments, the interaction between the target molecule and the test molecule is irreversible. In certain embodiments, the interaction between the target protein and the test molecule results in oligomerization of the target protein.

In certain embodiments, the methods, e.g., high throughput methods, of the present disclosure allow for identifying an interaction between a target molecule and a test molecule that induces a conformation change in the target molecule. For example, but not by way of limitation, methods of the present disclosure can be used to analyze a library of test molecules to identify the test molecules that cause a conformational change in the target molecule. For example, but not by way of limitation, methods of the present disclosure can identify test molecules that bind allosterically to the target molecule. In certain embodiments, methods of the present disclosure can identify test molecules that bind orthosterically to the target molecule. In certain embodiments, the methods can comprise: (a) contacting cell-free samples comprising a plurality of the target molecule with a plurality of test molecules, where each sample is contacted with a different test molecule; (b) tracking a plurality of the target molecules in each sample over time to provide a plurality of spatiotemporal trajectories and/or measurements of rotational motion; (c) analyzing the plurality of spatiotemporal trajectories and/or measurements of rotational motion to determine the target molecule's movement in the presence of the test molecules; and (d) comparing the target molecule's movement obtained in (c) with a reference target molecule's movement, wherein the reference target molecule's movement is the movement of the target molecule in the presence of a test molecule that does not induce a conformational change in the target molecule; wherein a change in the target molecule's movement compared to the reference target molecule's movement indicates an interaction between the target molecule and the test molecule that induces a conformational change in the target molecule. In certain embodiments, the duration and/or reversibility of the target molecule's movement change compared to the duration and/or reversibility of the reference target molecule's movement change indicates an interaction between the target molecule and the test molecule that induces a conformational change in the target molecule. In certain embodiments, the conformational change in the target molecule results in a change in the target molecule's temperature stability.

The present disclosure further includes methods, e.g., high throughput methods, for determining a dose response of a target molecule to a test molecule. For example, but not by way of limitation, the method can comprise: (a) contacting a plurality of cell-free samples comprising a plurality of the target molecule with the test molecule, where plurality of cell-free samples are contacted with a range of test molecule doses; (b) tracking a plurality of the target molecules over time in the presence of the range of doses of the test molecule to provide a plurality of spatiotemporal trajectories and/or measurements of rotational motion; (c) analyzing the plurality of spatiotemporal trajectories and/or measurements of rotational motion of the target molecules in the presence of the range of doses of the test molecule to determine the target molecule's movement at each dose of test molecule; (d) comparing the target molecule's movement obtained at different test molecule doses in (c) to determine the dose response of the target molecule to the test molecule.

The present disclosure further includes methods, e.g., high throughput methods, for determining a difference in dose response to a test molecule by two target molecules comprising: (a) contacting a first plurality of cell-free samples comprising a plurality of the first target molecule with the test molecule, where first plurality of cell-free samples are contacted with a range of test molecule doses; (b) tracking a plurality of the target molecules over time in the presence of the range of doses of the test molecule to provide a first plurality of spatiotemporal trajectories and/or measurements of rotational motion; (c) analyzing the plurality of spatiotemporal trajectories and/or measurements of rotational motion of the target molecules in the presence of the range of doses of the test molecule to determine the first target molecule's movement at each dose of test molecule; (d) comparing the target molecule's movement obtained at different test molecule doses in step (c) to determine the dose response of the target molecule to the test molecule; (e) repeating steps (a)-(d) with a second target molecule to determine the dose response of the second target molecule to the test molecule; and (f) comparing the dose response of the first target molecule to the dose response of the second target molecule to determine the difference in the response of the first and second target molecules to the test molecule. In certain embodiments, more than two target molecules can be analyzed, e.g., three target molecules.

In certain embodiments, the present disclosure provides methods, e.g., high throughput methods, for identifying an interaction between a test molecule and a target protein comprising a disordered domain. In certain embodiments, the target protein does not comprise an structured domain. In certain embodiments, the methods comprise: (a) contacting a cell-free sample comprising a plurality of the target protein with a plurality of the test molecule; (b) tracking a plurality of the target protein over time to provide a plurality of spatiotemporal trajectories and/or measurements of rotational motion; (c) analyzing the plurality of spatiotemporal trajectories and/or measurements of rotational motion to determine the target protein's movement in the presence of the test molecule; and (d) comparing the target protein's movement obtained in (c) with a reference target protein's movement, wherein the reference target protein's movement is the movement of the target protein in the absence of the test molecule; wherein a change in the movement of the target protein comprising the disordered domain compared to the reference target protein's movement indicates an interaction between the target protein comprising the disordered domain and the test molecule. In certain embodiments, the duration and/or reversibility of the movement change of the target protein comprising the disordered domain compared to the duration and/or reversibility of the reference target protein's movement change indicates an interaction between the target protein comprising the disordered domain and the test molecule. In certain embodiments, the interaction between the target protein comprising the disordered domain and the test molecule is reversible. In certain embodiments, the interaction between the target protein comprising the disordered domain and the test molecule is irreversible. In certain embodiments, a decrease in the diffusion coefficient of the target protein comprising the disordered domain compared to the reference target protein diffusion coefficient indicates an interaction between the target protein comprising the disordered domain and the test molecule. In certain embodiments, an increase in the diffusion coefficient of the target protein comprising the disordered domain compared to the reference target protein diffusion coefficient indicates an interaction between the target protein comprising the disordered domain and the test molecule.

The present disclosure further provides methods, e.g., high throughput methods, for identifying a test molecule that can distinguish between at least two target molecules. In certain embodiments, the methods comprise: (a) contacting a cell-free sample comprising a plurality of a first target molecule with a plurality of the test molecule; (b) tracking a plurality of the first target molecule over time to provide a plurality of spatiotemporal trajectories and/or measurements of rotational motion; (c) analyzing the plurality of spatiotemporal trajectories and/or measurements of rotational motion to determine the first target molecule's movement in the presence of the test molecule; (d) contacting a cell-free sample comprising a plurality of a second target molecule with a plurality of the test molecule; (e) tracking a plurality of the second target molecule over time to provide a plurality of spatiotemporal trajectories and/or measurements of rotational motion; (f) analyzing the plurality of spatiotemporal trajectories and/or measurements of rotational motion to determine the movement of the second target molecule in the presence of the test molecule; and (g) comparing the target molecule's movement obtained in (c) and (f); wherein a change in the movement of the first and second target molecule indicates that the test molecule can distinguish between the two target molecules. In certain embodiments, distinguishing between the first and second target molecule is achieved by differences in their fluorescent labels. In certain embodiments, more than two target molecules can be analyzed, e.g., three target molecules. For example, but not by way of limitation, the method can further include contacting a cell-free sample comprising a plurality of a third target molecule with a plurality of the test molecule; tracking a plurality of the third target molecules over time to provide a third plurality of spatiotemporal trajectories and/or measurements of rotational motion; analyzing the third plurality of spatiotemporal trajectories and/or measurements of rotational motion to determine the movement of the third target molecules in the presence of the test molecule; and comparing the third target molecule's movement to the movements obtained in (c) and (f); wherein a change in the movement of the third target molecule relative to the first and second target molecules indicates that the test molecule can distinguish between the three target molecules.

The present disclosure further provides methods, e.g., high throughput methods, for identifying a test molecule that can distinguish between at least two target molecules. In certain embodiments, the methods comprise: (a) contacting a cell-free sample comprising a plurality of a first target molecule and a plurality of a second target molecule with a plurality of the test molecule; (b) tracking a plurality of the first target molecules over time to provide a plurality of spatiotemporal trajectories and/or measurements of rotational motion; (c) analyzing the plurality of spatiotemporal trajectories and/or measurements of rotational motion to determine the first target molecule's movement in the presence of the test molecule; (d) tracking a plurality of the second target molecules over time to provide a plurality of spatiotemporal trajectories and/or measurements of rotational motion; (e) analyzing the plurality of spatiotemporal trajectories and/or measurements of rotational motion to determine the second target molecule's movement in the presence of the test molecule; and (f) comparing the first and second target molecules' movement obtained in (c) and (f); wherein a change in the movement of the first and second target molecule indicates that the test molecule can distinguish between the two target molecules.

### 3.4 Single Molecule Fluorescence Polarization

The present disclosure further provides methods for characterizing the molecular dynamics of a target molecule by exploiting the orientation-dependent fluorescence properties of fluorophores sufficiently rigidly attached to the target molecule. In certain embodiments, the disclosed methods comprise detecting changes in fluorophore orientation and rotational freedom upon oligomer formation. In certain embodiments, the disclosed methods comprise detecting how conformational changes affect the orientation and dynamics of fluorophores attached to specific sites within a molecule. In certain embodiments, the disclosed methods comprise characterizing dynamic structural transitions, such as protein folding-unfolding events or nucleic acid conformational changes. In certain embodiments, the disclosed methods comprise time-resolved anisotrophy measurements and polarization-sensitive imaging techniques coupled with advanced optical controls. In certain embodiments, the disclosed methods comprise (a) Time-Correlated Single-Photon Counting (TCSPC), (b) step-scan pump-probe techniques, (c) ultra-long lifetime fluorophores, and/or (d) advanced optical techniques.

In certain embodiments, TCSPC is used to detect individual photon arrival times with picosecond accuracy. In certain embodiments, TCSPC comprises 2D TCSPC via Single Photon Avalanche Diode (SPAD) arrays and photon-counting modules. In certain embodiments, TCSPC is used to resolve dynamics on a nanosecond scale. In certain embodiments, TCSPC is paired with polarization-sensitive readouts to study fast rotational dynamics and short fluorescence lifetimes typical of many biological fluorophores. In certain embodiments, TCSPC is used to discriminate between different fluorophore states and/or orientations.

In certain embodiments, step-scan pump-probe techniques comprise modulating the polarization of excitation and probing light and the resulting fluorescence emission over time provides insights into the orientation/rotation and dynamics of fluorophores. In certain embodiments, step-scan pump-probe techniques comprises using step-scan pump-probe spectromicroscopy to measure polarization-dependent and time-correlated fluorescence depletion dynamics. In certain embodiments, step-scan pump-probe techniques are used for optically filtering subpopulations of a distinct molecular size, orientation, or dynamic, and/or for studying dynamics in systems where fluorophore orientation changes rapidly post-excitation, e.g., in protein dynamics or structural changes in membranes.

In certain embodiments, ultra-long lifetime fluorophores exhibit fluorescence lifetimes longer than typical fluorescence decay times. In certain embodiments, ultra-long lifetime fluorophores exhibit fluorescence lifetimes microseconds longer than typical fluorescence decay times. In certain embodiments, ultra-long lifetime fluorophores exhibit fluorescence lifetimes nanoseconds longer than typical fluorescence decay times. In certain embodiments, ultra-long lifetime fluorophores are used to extend depolarization times significantly beyond typical fluorescent times. Non-limiting examples of ultra-long lifetime fluorophores are carbon dots, quantum dots, and nanodiamonds. In certain embodiments, ultra-long lifetime fluorophores are used to measure rotational dynamics and depolarization processes over extended timescales.

In certain embodiments, advanced optical techniques are used for precise spatial manipulation and control of light polarization states. In certain embodiments, advanced optical techniques comprise wobble anisotropy and polarization filtering. In certain embodiments, advanced optical techniques comprise advanced optical methodologies involving phase and polarization control. In certain embodiments, advanced optical techniques are used to achieve high-precision polarization-dependent measurements.

### 4. SYSTEMS FOR BIOPHYSICAL ANALYSIS

### 4.1 Image Acquisition Systems

In certain embodiments, aspects of the current subject matter can be implemented using a SMT workflow, e.g., an htSMT workflow, where such workflow incorporates systems for image acquisition. For example, such image acquisition can incorporate the imaging of samples to generate a series of images and/or videos. In certain embodiments, the exemplary image acquisition system comprises: a light source configured to emit light relayed by one or more optical elements in an optical relay, the optical relay being configured to shape the light emitted from the light source to form a shaped beam such that the shaped beam has a uniform intensity across a longer dimension of the linear shape; an optical element, e.g., a galvo mirror, configured to translate the shaped beam, and which can be position either before or after the optical relay configured to form a shaped beam such that the shaped beam has a uniform intensity across a longer dimension of the linear shape; and one or more optical elements, e.g., a dichroic mirror (2-100), configured to direct the shaped beam to an objective, whereby a portion of the sample plane is illuminated by an inclined beam, resulting in the emission of light from the sample, e.g., fluorescence emission, which is focused by the objective, through a series of optical elements, e.g., a lens and an emission filter, to an image collection system.

In certain embodiments, a microscopy system for use performing the methods of the present disclosure can include (a) a stage for supporting a cell-free sample, wherein the cell-free sample comprises a target molecule, e.g., fluorescent molecule, (b) a light source for emitting a light beam capable of inducing a light-based response from the target molecule, e.g., fluorescent molecule, in the cell-free sample, (c) an objective for focusing the light beam on the cell-free sample in the sample plane, wherein the target molecule, e.g., fluorescent molecule, in the sample is disposed in a field of view in the sample plane, (d) a detector device for monitoring the light-based response from the target molecule, e.g., fluorescent molecule, over a period of time (e.g., in the presence of a test molecule), (e) a memory and (f) a processor in communication with the memory and the detector device. In certain embodiments, the processor is capable of determining a diffusion coefficient of the target molecule, e.g., fluorescent molecule, and comparing the diffusion coefficient with a reference diffusion coefficient. In certain embodiments, the processor is capable of determining a diffusion coefficient of the target molecule, e.g., fluorescent molecule, in the presence of a test molecule. In certain embodiments, the reference diffusion coefficient is a diffusion coefficient of the target molecule, e.g., fluorescent molecule, in the absence of the test molecule.

### 4.2 Light Source

In certain embodiments, a system of the present disclosure includes a light source configured to emit light. The light source, in certain implementations of the image acquisition systems disclosed herein, can be configured to emit light of a single wavelength. In certain implementations of the image acquisition systems disclosed herein, the light source can be configured to emit light of two, three, four, five, or more individual wavelengths. In certain implementations, the wavelength(s) of light emitted by the light source are predetermined. For example, but not by way of limitation, the wavelength(s) can be predetermined such that the emitted light elicits fluorescence emission when illuminating a sample, e.g., a sample comprising a fluorescent molecule, e.g., a fluorescent target molecule and/or a fluorescent test molecule. In certain embodiments, the wavelength(s) employed in connection with the methods described herein will fall within a range of 400 nm to 900 nm. In certain embodiments, the light source will emit light having a wavelength between 400 nm to 408, between 550 nm to 565 nm, or between 638 nm to 650 nm. In certain non-limiting implementations, the light source is configured to comprise three lasers with nominal central wavelengths 405 nm, 560 nm, 640 nm that could vary within absorption band of the fluorophores used. In certain embodiments, a 560 nm wavelength is used to excite dyes (e.g., JF549) attached to HaloTag or SortaseTag. In certain instances, a 642 or a 646 nm wavelength is used to excite dyes (e.g., JF₆₄₆) attached to HaloTag.

In certain non-limiting implementations, the light source is used to catalyze photochemical reactions. For example, but not by way of limitation, the wavelength(s) and illumination intensities can be such that cleavage of a chemical bond occurs. As an additional example, but not by way of limitation, the wavelength(s) and illumination intensities may induce the adoption of a non-radiative dark state (i.e., “photobleached molecule”). As an additional example, but not by way of limitation, the wavelength(s) and illumination intensities may induce radiative or non-radiative energy transfer between fluorophores within the sample.

In certain implementations of the image acquisition systems described herein, the light source can be configured to deliver a predetermined amount of power to the back focal plane of the objective. For example, but not by way of limitation, the light source delivers greater than 10 mW with respect to certain wavelengths, e.g., 405 nm, and/or greater than 150 mW with respect to other wavelengths, e.g., 640 nm. Additionally, or alternatively, in instances where the light source comprises three lasers emitting at 405 nm, 560 nm, and 640 nm wavelengths, respectively the light source can be configured to deliver predetermined amounts of power, to the back focal plane of the objective. For example, but not by way of limitation the 405 nm can be configured to deliver >10 mW; the 560 nm can be configured to deliver >150 mW; and the 640 nm can be configured to deliver >50 mW).

In certain implementations of the image acquisition systems described herein, the light source is configured to emit pulsed light. For example, but not by way of limitation, the light source can be configured to emit stroboscopic pulsed light. In certain implementations of the image acquisition systems described herein, the light source is configured to emit pulsed light in synchrony with the start of image acquisition. In certain, non-limiting implementations, the light source will pulse at specific time intervals depending on the number of frames per second being captured. For example, but not by way of limitation, in a 200 frame per second (FPS) mode, the laser is ON for 4 ms OFF for 1 ms. In certain implementations of the SMT workflow, the light source is configured to go from 90% to 10% power in less than about 0.4 ms. In certain implementations of the SMT workflow, the light source is configured to go from 90% to 10% power in less than about 0.2 ms. In certain embodiments, the light source will be configured to achieve 200, 400, 600, or 800 FPS.

The emission of light by the light source and the direction of that light to the optical relay, can, in certain implementations of the image acquisition systems disclosed herein, be facilitated using a single mode fiber. Alternatively, a multimode fiber can be employed in certain implementations of the image acquisition systems disclosed herein. For example, but not by way of limitation, the multimode fiber can be configured with a predetermined shape for sample illumination.

In certain implementations of the image acquisition systems described herein, for example with respect to systems configured for high throughput sample analysis, the light source can be configured to exhibit low drift in power output. In certain implementations, such low drift configurations increase sample processing consistency to facilitate high throughout analyses. For example, but not by way of limitation, such low drift power output configurations maintain power output within about 0% to about 15% variation, about 0% to about 10% variation, about 10% variation, about 9% variation, about 8% variation, about 7% variation, about 6% variation, about 5% variation, about 4% variation, about 3% variation, about 2% variation or about 1% variation.

In certain embodiments, such low drift power output configurations maintain power output within about 0% to about 15% variation, about 0% to about 10% variation, about 10% variation, about 9% variation, about 8% variation, about 7% variation, about 6% variation, about 5% variation, about 4% variation, about 3% variation, about 2% variation or about 1% variation in the context of changing ambient (room) temperature, e.g., 17° C.+/−5° C. In certain embodiments, this is achieved using temperature sensors and/or close-loop heaters to maintain internal light source (e.g., laser engine) temperatures stable, thereby reducing output power drift. For example, but not by way of limitation, the light source can be thermally insulated from the fluctuations of the ambient temperature using an insulated enclosure design. Additionally, or alternatively, closed-loop heaters can be strategically placed at specific locations in the system, e.g., the fiber coupler to reduce output drift. Additionally, or alternatively, water jackets and/or chillers can be used to reduce heat build-up from the laser heads. Moreover, these thermal controls, used individually or in combination, result in shorter warm up times to reach operating steady state and maintained more stable internal operating temperatures when lasers would be powered off and on.

### 4.3 Optical Elements & Sample Illumination

In certain embodiments, a system of the present disclosure includes a light source configured to emit light, which is relayed by one or more optical elements in an optical relay, the optical relay being configured to shape the light emitted from the light source to form a shaped beam. The particular optical elements of any particular optical relay implementation can be selected and configured to produce the appropriately shaped beam as well as provide for the appropriate translation of that beam.

In certain, non-limiting, implementations of the optical relay of the presently disclosed image acquisition systems, the optical relay will comprise one or more lenses and/or other optical elements. For example, but not by way of limitation, the selection and orientation of lenses and other optical elements in the optical relay will be configured to appropriately shape the light beam being directed to the sample. In certain non-limiting implementations, the optical relay will comprise optical elements to collimate the emitted light, e.g., a collimator, from the light source. Additionally, or alternatively, the optical relay will comprise additional optical elements, e.g., a Powell lens or other elements adapted to produce a beam fan, one or more cylinder lenses, one or more slits to adjust light sheet extent, one or more achromatic lenses and/or one or more mirrors, one or more of which can be a galvo mirror capable of translating the light. The particular attributes of the optical element will be predetermined to produce an appropriately shaped light beam. For example, but not by way of limitation, the SMT systems of the present disclosure can achieve uniform horizontal FOV as well as uniform vertical FOV. Such uniformity in horizontal and vertical FOVs contrasts with other strategies that provide non-uniform horizontal FOV and/or non-uniform vertical FOV.

To achieve uniform horizontal FOV as well as uniform vertical FOV, the optical relay of the SMT systems described herein comprise an optical element or assembly capable of producing a beam that is elongated along the X plane, and narrow along Y plane and wherein the light beam has a uniform intensity across a longer dimension of the linear shape. In certain non-limiting implementations, the optical relay of the SMT systems described herein will comprise a Powell lens to shape the light beam such that it has a uniform intensity across a longer dimension of the linear shape. The optical relay of the SMT systems described herein can comprise additional or alternative optical elements or assemblies to shape the light beam such that it has a uniform intensity across a longer dimension of the linear shape. For example, but not by way of limitation, the optical relay of the SMT systems described herein can comprise a diffraction element or assembly configured to shape the light beam such that it has a uniform intensity across a longer dimension of the linear shape.

In certain, non-limiting implementations of the optical relays of the presently disclosed image acquisition systems, the optical relay will comprise one or more optical elements or assemblies configured to translate the light beam relative to the sample plane of the sample to be analyzed, e.g., in a direction orthogonal to the longer dimension of the light beam. For example, but not by way of limitation, such optical elements or assemblies configured to translate the light beam relative to the sample plane of the sample to be analyzed can comprise a galvo mirror or a piezo element configured to translate the light beam. Additionally, or alternatively, such optical elements or assemblies configured to translate the light beam relative to the sample plane of the sample to be analyzed can comprise a computer-controlled motor. In certain embodiments, the optical element or assemblies configured to translate the light beam can be positioned before or after optical element or assembly, e.g., a Powell lens, configured to shape the light beam such that it has a uniform intensity across a longer dimension of the linear shape.

In certain embodiments, the system comprises an optical relay configured to shape the light emitted from the light source to form a shaped beam, which is then directed by an optical element, e.g., a dichroic mirror, configured to direct the shaped beam to an objective, whereby the sample plane is illuminated by an inclined beam.

In certain, non-limiting implementations of the image acquisition systems of the present disclosure, an objective directs the inclined beam on the sample plane to be analyzed. In certain, non-limiting implementations of the image acquisition systems of the present disclosure the objective is a water immersion objective. The use of a water immersion objective facilitates high throughput sample analysis by eliminating the oil present in connection with the use of oil immersion objectives, thereby allowing for higher image quality and less distortion. The presence of oil present issues in the context of automated systems, where the oil can spread to components, including optical elements that can be fouled by exposure to oil, water-immersion objectives are better index-matched for imaging aqueous samples. In certain, non-limiting implementations, the objective 60×1.27 NA water immersion objective (Nikon). In certain implementations of the workflows described herein, the water immersion objective will be heated by a heating element. For example, such heating element will maintain the water immersion objective at a temperature sufficient to avoid inducing a change in temperature of the sample contained in the sample plate.

### 4.4. Image Acquisition

In certain, non-limiting implementations of the image acquisition systems of the present disclosure, the objective is also used to focus the fluorescence emitted by the sample in response to the illumination provided by the inclined beam. In certain, non-limiting implementations, the objective-focused fluorescence emission is passed through an emission filter, e.g., a bandpass emission filter matched to the spectrum of the fluorophore under observation and mounted in high-speed filter wheel (Finger Lakes Instruments), and collected by a detector device. In certain, non-limiting implementations, the objective-focused fluorescence emission is directed to an optical relay prior to collection by the detector device. For example, but not by way of limitation, such an optical relay can comprise one or more lenses and one or more additional optical elements, e.g., an element configured to reject additional scattered light, prior to collection by the detector device. In certain, non-limiting implementations, the objective-focused fluorescence emission is directed through another diachroic mirror to split the emission over multiple regions of the detector. In certain, non-limiting implementations, the objective-focused fluorescence emission is directed through another diachroic mirror to split the emission over multiple detectors.

In certain, non-limiting implementations of the image acquisition systems of the present disclosure, the detector device is configured to synchronize detection with the translation of the inclined beam across the sample plane. For example, but not by way of limitation, the detector device can be a CMOS camera, e.g., a back illuminated CMOS camera (the Hamamatsu Fusion BT).

In certain implementations of the image acquisition systems of the present disclosure, the CMOS camera can be run such that, for each field of view, a series of SMT frames are collected. For example, but not by way of limitation, 1-20,000 SMT frames, 1-15,000 SMT frames, 1-10,000 SMT frames, 1-5,000 SMT frames, 1-1,000 SMT frames, 2-500 SMT frames, 5-250 SMT frames, 10-200 SMT frames, 100-200 SMT frames, or 200 SMT frames are collected per field of view. In certain implementations, the CMOS camera can be configured to run at a frame rate from about 0.5 to about 2000 Hz. In certain implementations, the CMOS camera can be configured to run at a frame rate of from 0.5 to 1000 Hz or in certain implementations, at 200 Hz. In certain embodiments, the CMOS camera can be configured to run at a frame rate of from 100 Hz to 1250 Hz. For example, but not by way of limitation, certain SMT implementations can be performed at 200 Hz. In certain embodiments, certain SMT implementations can be performed at 400 Hz. In certain embodiments, certain SMT implementations can be performed at 800 Hz. In certain embodiments, certain SMT implementations can be performed at 1000 Hz. In certain embodiments, certain SMT implementations can be performed at 1200 Hz. In certain embodiments, certain SMT implementations can be performed at 1250 Hz. In certain embodiments, certain SMT implementations can be performed at 1400 Hz. In certain embodiments, certain SMT implementations can be performed at 1600 Hz. In certain embodiments, certain SMT implementations can be performed at 1800 Hz. In certain embodiments, certain SMT implementations can be performed at 2000 Hz. In certain embodiments, certain SMT implementations can be performed at a frame rate of about 100 Hz or higher, about 200 Hz or higher, about 400 Hz or higher, about 600 Hz or higher, about 800 Hz or higher, about 1000 Hz or higher, about 1200 Hz or higher, about 1400 Hz or higher, about 1600 Hz or higher or about 1800 Hz or higher. In certain embodiments, certain SMT implementations can be performed at a frame rate up to about 1200 Hz. In certain embodiments, certain SMT implementations can be performed at a frame rate up to about 1400 Hz. In certain embodiments, certain SMT implementations can be performed at a frame rate up to about 1600 Hz. In certain embodiments, certain SMT implementations can be performed at a frame rate up to about 1800 Hz. In certain embodiments, certain SMT implementations can be performed at a frame rate up to about 2000 Hz. In certain embodiments, the CMOS camera will be configured to achieve 200, 400, or 800 FPS.

In certain, non-limiting implementations of the image acquisition systems of the present disclosure, the detector device is configured to transmit a signal with each frame to trigger other elements of the imaging system. For example, but not by way of limitation, the detector device may trigger the illumination from the light source so as to collect fluorescence emission associated with stroboscopic laser pulses. For example, but not by way of limitation, such fluorescence emission collection is associated with 10 to 100 ms frames and a 2 ms stroboscopic laser pulse. In certain embodiments, fluorescence emission collection is associated with a stroboscopic laser pulse of about 0.1 to about 1 ms. In certain embodiments, fluorescence emission collection is associated with a stroboscopic laser pulse of about 0.2 to about 1 ms, of about 0.3 to about 1 ms, of about 0.4 to about 1 ms, of about 0.1 to about 0.9 ms, of about 0.1 to about 0.8 msec, of about 0.1 to about 0.7 ms, of about 0.1 to about 0.6 ms, of about 0.1 to about 0.5 ms, of about 0.1 to about 0.4 ms, of about 0.2 to about 0.6 ms, of about 0.2 to about 0.5 ms, of about 0.2 to about 0.4 ms or of about 0.3 to about 0.5 ms. In certain embodiments, fluorescence emission collection is associated with a stroboscopic laser pulse of about 0.1 to about 0.6 ms. In certain embodiments, fluorescence emission collection is associated with a stroboscopic laser pulse of about 0.1 to about 0.5 ms. In certain embodiments, fluorescence emission collection is associated with a stroboscopic laser pulse of about 0.2 to about 0.4 ms. In certain embodiments, fluorescence emission collection is associated with a stroboscopic laser pulse of about 0.2 ms. In certain embodiments, fluorescence emission collection is associated with a stroboscopic laser pulse of about 0.4 ms.

In certain implementations, the imaging acquisition system can be configured to acquire a predetermined field of view (FOV), e.g., a detected FOV. In certain embodiments, the FOV, e.g., detected FOV, can have a size of about 150 μm to about 250 μm in a first dimension by about 100 μm to about 210 μm in a second dimension. In certain embodiments, the FOV, e.g., detected FOV, can have a size of about 200 μm to about 250 μm in a first dimension by about 150 μm to about 210 μm in a second dimension or the FOV, e.g., detected FOV, can have a size of about 225 μm to about 250 μm in a first dimension by about 175 μm to about 210 μm in a second dimension. For example, but not by way of limitation, the FOV, e.g., detected FOV, can have a size of about 250 μm in a first dimension by about 190 μm in a second dimension, e.g., as disclosed in Example 1.

In certain embodiments, a certain percentage of an FOV, e.g., a detected FOV, provides useable data. In certain embodiments, at least 75% of the FOV, at least 80% of the FOV, at least 85% of the FOV, at least 90% of the FOV, at least 95% of the FOV, at least 96% of the FOV, at least 97% of the FOV, at least 98% of the FOV, at least 99% of the FOV or 100% of the FOV provides useable data. In certain embodiments, at least 75% of the FOV, e.g., detected FOV, provides useable data. In certain embodiments, at least 80% of the FOV, e.g., detected FOV, provides useable data. In certain embodiments, at least 85% of the FOV, e.g., detected FOV, provides useable data. In certain embodiments, at least 90% of the FOV, e.g., detected FOV, provides useable data. In certain embodiments, at least 95% of the FOV, e.g., detected FOV, provides useable data. In certain embodiments, at least 96% of the FOV, e.g., detected FOV, provides useable data. In certain embodiments, at least 97% of the FOV, e.g., detected FOV, provides useable data. In certain embodiments, at least 98% of the FOV, e.g., detected FOV, provides useable data. In certain embodiments, at least 99% of the FOV, e.g., detected FOV, provides useable data. In certain embodiments, 100% of the FOV, e.g., detected FOV, provides useable data. In certain embodiments, a percentage equal to or greater than about 75% of the FOV provides useable data, e.g., a percentage equal to or greater than about 80% of the FOV, a percentage equal to or greater than about 85% of the FOV, a percentage equal to or greater than about 90% of the FOV, a percentage equal to or greater than about 95% of the FOV, a percentage equal to or greater than about 96% of the FOV, a percentage equal to or greater than about 97% of the FOV, a percentage equal to or greater than about 98% of the FOV or a percentage equal to or greater than about 99% of the FOV provides useable data. In certain embodiments, a certain percentage of an FOV, e.g., a detected FOV, achieves sufficient laser illumination for tracking protein movement. For example, but not by way of limitation, at least 75% of the FOV, at least 80% of the FOV, at least 85% of the FOV, at least 90% of the FOV, at least 95% of the FOV, at least 96% of the FOV, at least 97% of the FOV, at least 98% of the FOV, at least 99% of the FOV or 100% of the FOV achieves sufficient laser illumination for tracking protein movement. In certain embodiments, at least 75% of the FOV, e.g., detected FOV, achieves sufficient laser illumination for tracking protein movement. In certain embodiments, at least 80% of the FOV, e.g., detected FOV, achieves sufficient laser illumination for tracking protein movement. In certain embodiments, at least 85% of the FOV, e.g., detected FOV, achieves sufficient laser illumination for tracking protein movement. In certain embodiments, at least 90% of the FOV, e.g., detected FOV, achieves sufficient laser illumination for tracking protein movement. In certain embodiments, at least 95% of the FOV, e.g., detected FOV, achieves sufficient laser illumination for tracking protein movement. In certain embodiments, at least 96% of the FOV, e.g., detected FOV, achieves sufficient laser illumination for tracking protein movement. In certain embodiments, at least 97% of the FOV, e.g., detected FOV, achieves sufficient laser illumination for tracking protein movement. In certain embodiments, at least 98% of the FOV, e.g., detected FOV, achieves sufficient laser illumination for tracking protein movement. In certain embodiments, at least 99% of the FOV, e.g., detected FOV, achieves sufficient laser illumination for tracking protein movement. In certain embodiments, 100% of the FOV, e.g., detected FOV, achieves sufficient laser illumination for tracking protein movement. In certain embodiments, a percentage equal to or greater than about 75% of the FOV achieves sufficient laser illumination for tracking protein movement, e.g., a percentage equal to or greater than about 80% of the FOV, a percentage equal to or greater than about 85% of the FOV, a percentage equal to or greater than about 90% of the FOV, a percentage equal to or greater than about 95% of the FOV, a percentage equal to or greater than about 96% of the FOV, a percentage equal to or greater than about 97% of the FOV, a percentage equal to or greater than about 98% of the FOV or a percentage equal to or greater than about 99% of the FOV provides sufficient laser illumination for tracking protein movement.

In certain implementations, the imaging acquisition system can be configured to acquire a predetermined image dimension per frame, referred to herein as the Region of Interest (ROI). In certain implementations, the ROI will differ depending on the frame rate employed. For example, at, at 200 FPS, 2304×768 pixels will define the ROI, which amounts to 248.832×82.944 μm² in the sample plane; at 400 FPS, 2304×432 pixels with define the ROI, which amounts to 248.832×46.6 μm² in the sample plane; at 800 FPS, 2304×192 pixels, which amounts to 248.832×20.928 μm² in the sample plane. In certain embodiments, the imaging acquisition system can be configured to achieve 200, 400, or 800 FPS.

In certain implementations, the imaging acquisition system can be configured to perform predetermined sweep rates at predetermined frame rates. For example, but not by way of limitation, the sweep rate can be 82.94 μm/4 ms which is equivalent to 20.7 μm/ms which is equivalent to 2.07 cm/s. In certain embodiments, the imaging acquisition system can be configured to perform sweep rates to achieve 200, 400, 600, or 800 FPS.

In certain implementations, the detector device can be used to collect fluorescence emission at multiple wavelengths. For example, but not by way of limitation, fluorescence emission of additional fluorophores can be collected at the same frame rate or different frame rates for the same fields of view to provide downstream registration of SMT tracks to other labeled components. Additional channels of the detector device can be used as desired to expand the number of simultaneously captured fluorescence emissions for the same fields of view to provide downstream registration of SMT tracks to other components, e.g., test molecules, within the sample.

### 4.5 SMT Software

The present disclosure provides an example system for a high-throughput single-molecule imaging platform that measures molecule movement in cell-free samples. Experiments can be performed to collect large amounts of data from a plurality of target molecules. The experiments can include the application of various identifiers to target molecules such as labels which can be subsequently fluoresced or otherwise detected (e.g., using a laser or other light source). The samples forming part of such experiments can be organized into plates having a plurality of wells. Each well can have one or more associated fields of view (FOVs). FOVs can be locations within or corresponding to a single well. A sequence of images can be generated for the FOVs to result in one or more movies, which can include SMT movies as well as non-SMT movies. SMT movies can be used to track the paths of individual labeled molecules such as proteins, generating a plurality of spatiotemporal trajectories. Each spatiotemporal trajectory may be comprised of a plurality of spots, which include the spatiotemporal coordinates of a labeled molecule at a particular time. Separately from the tracking, and in some instances in parallel with the tracking, the movies can be utilized to identify molecules through the use of machine-learning and/or computer vision-based image segmentation to generate masks. Masks are spatial regions within a FOV produced by the segmentation. Each mask can belong to a mask category.

Tracking data and segmentation data can, in certain embodiments, be combined to generate a plurality of metrics associated with various aspects of the samples, e.g., the position of phase changes or other sample attributes. In other words, the spatiotemporal trajectories (e.g., spatiotemporal trajectory data) can be combined with the machine learning processed image segmentation data and further analyzed using statistical/machine learning methods. Processing of the combined data can be used to generate metrics such as hit scores associated with compounds and/or targets within a sample that may be stored in a database structure.

The present disclosure provides an exemplary data flow a high-throughput single-molecule imaging platform that measures protein movement in cell-free samples. Experiment specifications that define experiments can be provided as data input via one or more clients. The experiment specifications can define various parameters for the experiments such as dyes, compounds, treatments, and the like. In certain embodiments, an imaging system (e.g., imaging system) can capture a sequence of images that generate one or SMT movies and/or non-SMT movies or segmentation movies (e.g., movies) which characterize molecular movement. The SMT movies can characterize movement of individual fluorescent molecules and/or contain images of individual fluorescent molecules. The segmentation movies can comprise a sequence of images that characterize movement of labeled molecules and/or component thereof.

The SMT movies can be analyzed to perform operations relating to molecule tracking which can include detecting, subpixel localization, and linking to identify spatiotemporal trajectories of molecules across various images within the SMT movies. More specifically, during detection one or more spots within the SMT movies can be detected or recovered. Each spot can be equipped with spatiotemporal coordinates. These spatiotemporal coordinates can be estimated by using subpixel localization techniques. Linking can be performed on the spots to ultimately identify spatiotemporal trajectories.

Links, as used herein, are potential associations between two spots. Each link is directed, beginning at one spot and ending at another. A “correct link” joins two spots produced by the same emitter in different frames; otherwise, a link is “incorrect.” One objective of the linking algorithm is to estimate which links are correct. Links are referred to herein in the format a: i→j This is taken to mean: link a, which begins at spot i and ends at spot j. Links satisfy at least three of the following constraints: (a) links go forward in time, (b) links may not join two spots that are farther apart than some limit (referred to herein as the “search radius”), and (c) links may not join two spots that are temporally separated by more than some limit (referred to herein as the “gap limit”). A spot-link graph is a graph of spots and links for one SMT movie. The spots are the vertices and the links are the edges of this graph. Because links go forward in time, the spot-link graph is a directed acyclic graph. A matching is a subset of the links in a spot-link graph such that no two links in this subset begin or end at the same spot. Spatiotemporal trajectories 715 are used herein to refer to sequences of contiguous (end-to-end) links in the same matching. Dynamical metrics can be determined using a plurality of spatiotemporal trajectories. Such parameters can comprise attributes of a spot that characterize the spot's movement. Such parameters can comprise one or more of velocity, diffusion coefficient, or anomaly parameter(s) for each spot. The dynamical parameter(s) for spot i are herein referred to as θᵢ. The set of dynamical parameters for all spots in a spot-link graph are herein referred to as θ.

Experiment information such as the dynamical metrics, the image metrics, and any data from which either metric is derived (e.g., segmentation information) can be provided to a data repository for storage. Such data repository can store, for example, any results of experiment such as the dynamical metrics, image metrics, and/or any data from which either metric is derived. Data repository can comprise local persistence and/or dedicated servers accessed locally or by way of the cloud. Data repository can also store metadata associated therewith and/or metadata associated with the experiment specification. The experiment information (e.g., results and metadata from historical experiments, etc.) can be provided to data repository via a repository application program interface (API). The repository API can also interface with a web-based graphical user interface front end that provides such information for display on clients.

Example dynamical metrics can also include state arrays. State arrays are a framework for learning interpretable dynamical models from SMT spatiotemporal trajectories, and can be used for gaining additional insight into the movement of a target protein. In some variations, state arrays can be generated/populated using the segmentation information. State arrays can be used to quantify relative occupancies of a molecule in different conformations (e.g., open and closed). Fractional occupancies can be calculated by taking the average of each state array bin, weighted by each bin's occupancy, over the total weighted average diffusion coefficient.

In certain embodiments, the computer-implemented environment can include an imaging system that interacts with a computing architecture to perform the various algorithms described herein. The imaging system can interface with one or more clients (e.g., clients via a web application having a graphical user interface such). The one or more clients can interface with one or more servers accessible through the network(s). The one or more clients can host a frame grabber that captures images from a camera (e.g., movies). Those images can be temporarily stored on the one or more clients and periodically transferred to the one or more servers for remote storage via network. The one or more servers can also contain or have access to one or more data stores for storing data collected and/or extracted from a sample by imaging system. In some variations, the network may include or interface with one or more network storage arrays for storing data such as the captured images (e.g., movies).

In some variations, the sample computing device architecture can be that of client(s) and/or of server(s) and some components described in relation to diagram may be optional for the client(s) and/or servers(s). A bus can serve as the information highway interconnecting the other illustrated components of the hardware. A processing system labeled CPU (central processing unit) (e.g., one or more computer processors/data processors at a given computer or at multiple computers), can perform calculations and logic operations required to execute a program. Optionally or additionally, a processing system labeled GPU (graphics processing unit) (e.g., one or more computer processors/data processors at a given computer or at multiple computers), can perform calculations and logic operations required to execute a program. A non-transitory processor-readable storage medium, such as read only memory (ROM) and random access memory (RAM), can be in communication with the processing system and/or processing system and can include one or more programming instructions for the operations specified here. Optionally, program instructions can be stored on a non-transitory computer-readable storage medium such as a magnetic disk, optical disk, recordable memory device, flash memory, solid state drive or other physical storage medium.

In one example, a disk controller can interface with one or more optional removable storage or local storage to the system bus. The removable storage can be external or internal disk drives, or solid state drives, or external hard drives. The local storage can be internal hard drives and/or memory. As indicated previously, these various examples of removable storage, local storage, and disk controllers are optional devices. The system bus can also include at least one communications interface to allow for communication with external devices either physically connected to the computing system or available externally through a wired or wireless network such as cloud storage and remote services. In some cases, the at least one communications interface 1024 includes or otherwise comprises a network interface.

In some variations, such as for client(s), to provide for interaction with a user, the subject matter described herein can be implemented on a computing device having a display device (e.g., LCD (liquid crystal display) or LED (light-emitting diode) monitor) for displaying information obtained from the bus via a display interface to the user and an input device such as keyboard and/or a pointing device (e.g., a mouse or a trackball) and/or a touchscreen by which the user can provide input to the computer. Other kinds of input devices can be used to provide for interaction with a user as well; for example, feedback provided to the user can be any form of sensory feedback (e.g., visual feedback, auditory feedback by way of a microphone, or tactile feedback); and input from the user can be received in any form, including acoustic, speech, or tactile input. The input device and the microphone can be coupled to and convey information via the bus by way of an input device interface. By way of example, input device can be an imaging system configured with abilities to capture a sequence of images as described herein. A frame grabber can capture or grab individual frames from analog or digital data encapsulating the sequence of images obtained from the bus. Frame grabber may include memory that can store individual or multiple frames. Frame grabber can also provide individual or multiple frames to bus for further storage on, for example, local storage and/or removable storage.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

### 5. EXAMPLES

The presently disclosed subject matter will be better understood by reference to the following Examples, which are provided as exemplary of the presently disclosed subject matter, and not by way of limitation. This section describes the experimental materials and methods used for generating particular non-limiting exemplary data disclosed herein in Examples 1-5.

### Protein Expression and Purification:

The HaloTag expression vector (pH6HTN His6HaloTag® T7 Vectors) was purchased from Promega, and the expression vector for Halo-Protein 1 fusion was constructed by inserting sequence coding for Protein 1 to the C-terminus of HaloTag right after the TEV cleavage site. Both plasmids were transfected to E. coli BL21(DE3), grown in LB media at 37° C. to an of OD600 of 0.6-0.8, and induced at 16° C. overnight with 0.1 mM IPTG. Cells were harvested by centrifugation, lysed by BugBuster® protein extraction reagent (EMD Millipore), and proteins were purified by standard Ni-NTA methods. Eluted proteins were further purified by a size exclusion column pre-equilibrated with a buffer containing (25 mM HEPES, pH 7.9, 100 mM KCl, 12.5 mM MgCl₂, 0.1 mM EDTA, 10% glycerol, and 1 mM DTT). Pooled peak fractions were quantified by NanoDrop, aliquoted and snap frozen with liquid nitrogen for long term storage at −80° C.

Protein 2 was subcloned into pGEX4T-1 expression vector right after the Thrombin cleavage site, proceeded by a TEV cleavage site. Plasmids were transfected to E. coli BL21 (DE3), grow in LB media at 37° C. to OD600 of 0.6-0.8, and induced at 16° C. overnight with 0.1 mM IPTG. Cells were harvested by centrifugation, lysed by BugBuster® protein extraction reagent (EMD Millipore), and proteins were purified by standard glutathione agarose beads. GST tag was removed by Thrombin cleavage followed by a size exclusion column pre-equilibrated with a buffer containing (25 mM HEPES, pH 7.5, 150 mM NaCl, and 1 mM TCEP). Pooled peak fractions were quantified by NanoDrop, supplemented with glycerol to 10% v/v, aliquoted and snap frozen with liquid nitrogen for long term storage at −80° C.

DNA encoding Protein 3 and Protein 3 mutant were cloned into the pET11a bacterial expression vector with a N-terminal 6× His tag followed by TEV cleavage site and three additional glycine residues (a “GGGG” sequence will be left at the N-terminus after TEV cleavage). Sequenced plasmid was transformed into chemically competent E. coli (BL21DE3). Post single colony inoculation, bacteria were grown in LB at 37° C. until OD600 reaches 0.6-0.8 where 0.5 mM IPTG was added to induce for protein expression overnight at 16° C. Bacterial culture was resuspended in buffer (25 mM HEPES, pH 8.0, 150 mM NaCl, 1 mM TCEP, 5% glycerol) with freshly supplemented protease inhibitor then harvested, mechanically sonicated prior to incubating with a Ni-NTA affinity resin for batch purification. Proteins were washed with buffer containing 45 mM imidazole and eluted with lysis buffer containing 300 mM imidazole. TEV protease was used to cleave off the affinity tag overnight for the subsequent reverse affinity purification to yield the cleaved Protein 3 and Protein 3 mutant. Final proteins were further purified in size exclusion chromatography to buffer exchange (25 mM HEPES, pH 7.5, 150 mM NaCl). Peak fractions were pooled and supplemented with glycerol to 5% and stored at −80° C. after snap freezing with liquid nitrogen.

The protein sequence of Protein 4 was subcloned into pET17b vector with a N-terminal 6× His-tag. Transformed (BL21-DE3) E. coli were grown in Turbo Broth at 37° C. until OD600 reached 0.6. 0.1 mM IPTG was added to induce for protein expression at room temperature for an additional 4 hours. Harvested pellet was lysed in buffer (50 mM HEPES, pH 7.5, 150 mM NaCl, 1 mM TCEP+25 mM imidazole, 1 mM PMSF, supplemented with benzonase nuclease, and protease inhibitor) and loaded onto Ni-NTA column pre-equilibrated with the lysis buffer. Protein was eluted using lysis buffer supplemented with 500 mM imidazole and subsequently injected into HiTrap Q-FF column pre-equilibrated with buffer (50 mM HEPES, pH 8.0, 150 mM NaCl, 1 mM TCEP) and S75 Superdex size exclusion column in buffer (50 mM HEPES, pH 8.0, 150 mM NaCl, 1 mM TCEP) for further purification. Peak fractions were pooled and supplemented with glycerol to 5% and stored at −80° C. after snap freezing with liquid nitrogen.

The protein sequence encoding for Protein 5 was subcloned into pFASTBAC1 insect expression vector with an N-terminal GST tag and TEV cleavage site and C-terminal AviTag and StrepTag II affinity tag. This donor plasmid subsequently was transformed into DH10Bac E. Coli for bacmid integration and plated on Bluo-gal selection agar plate (Kan, Gen, Tet) for blue/white colony selection with the proper gene insertion product verified via PCR. To generate the baculovirus, bacmid was scaled up in LB and purified via Bacmid Maxiprep kit (Machery-Nagel) and pre-complexed with ExpiSf Expifectamine reagent (ThermoFisher Scientific) to transiently transfected ExpiSf9 cell (ThermoFisher Scientific). The harvested baculovirus was infected with ExpiSf9 at 1:50 dilution to induce for protein expression. The cell pellet was harvested and lysed using Insect Popculture detergent (Millipore Sigma) diluted in lysis buffer (50 mM HEPE, pH 8.0, 500 mM NaCl, 1 mM EDTA, 1 mM TCEP, 1 mM PMSF, supplemented with benzonase nuclease and protease inhibitor) at room temperature for at least 30 mins. Following lysate clarification, the soluble fraction was loaded onto StrepTactin affinity column (IBA Biosciences), MonoQ anion exchange column (Cytiva) and S200 Superdex size exclusion column (Cytiva) to remove aggregation and contaminants and exchange to a buffer containing (50 mM HEPES, pH 8.0, 250 mM NaCl, 1 mM TCEP). Pooled peak fractions were supplemented with glycerol to 10% and stored at −80° C. after snap freezing with liquid nitrogen.

### Protein Labeling:

Halo-tagged proteins 10˜60 μM were mixed with 100˜200 μM Halo-JF549 dyes (Tocris or in-house synthesized) in a buffer containing (50 mM HEPES pH 7.4, 150 mM NaCl, 0.01% NP40 alternative, 0.5 mM EDTA and 1 mM DTT), and incubated at room temperature (23° C.) for 15˜30 minutes. Free dyes were removed by a size exclusion column pre-equilibrated with a buffer (25 mM HEPES pH 7.9, 100 mM KCl, 12.5 mM MgCl₂, 0.1 mM EDTA, 10% glycerol, and 1 mM DTT). Pooled peak fractions were quantified by NanoDrop, aliquoted and snap frozen with liquid nitrogen for long term storage at −80° C.

For sortase based enzymatic ligation, the N-terminus is either cleaved via thrombin protease or TEV protease to expose the free Gly residues for labelling. Cleaved product was then incubated in labeling buffer (50 mM HEPE pH 7.5, 150 mM NaCl, 10 mM CalCl₂), 5 μM of recombinant purified His-tagged sortase (SrtAstaph pentamutant, DOI:10.1002/cpps.38), and 10 molar in excess (peptide donor to protein substrate) of LPETGG-JF549 peptide (JF549 conjugated to N-terminus of peptide LPETGG, Elim Biopharm) dissolved in water. After room temperature incubation at least 30-60 minutes, the reaction is cleaned up via magnetic separation using DYNABEADS™ His-tag and subsequently injected into size exclusion column for further separation of the labelled product and peptide-dye and exchange buffer (50 mM HEPES, pH 8.0, 150 mM NaCl, 1 mM TCEP). The ratio of the absorptions at 549 nm and 280 nm was used to estimate the labelling efficiency (40%-70%). The final product was supplemented with glycerol to 10% v/v and stored at −80° C.

### Single-Molecule Tracking and Data Analysis:

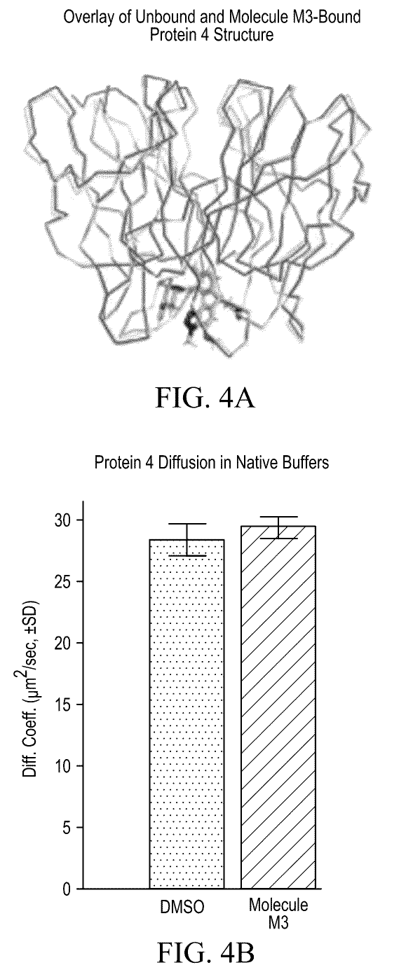

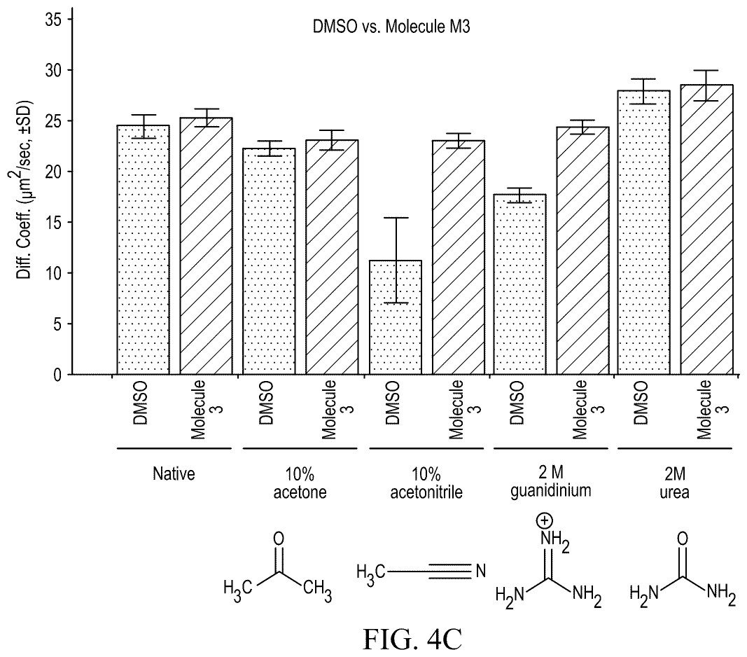

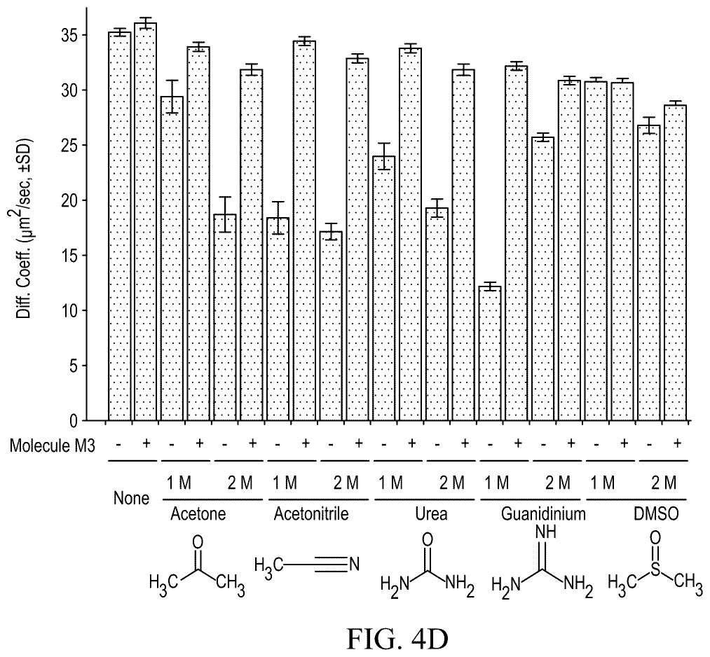

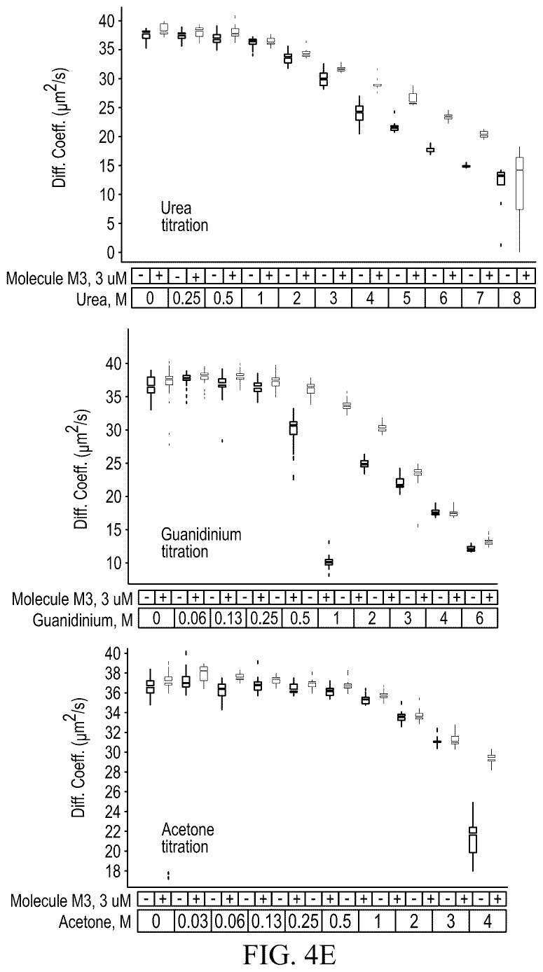

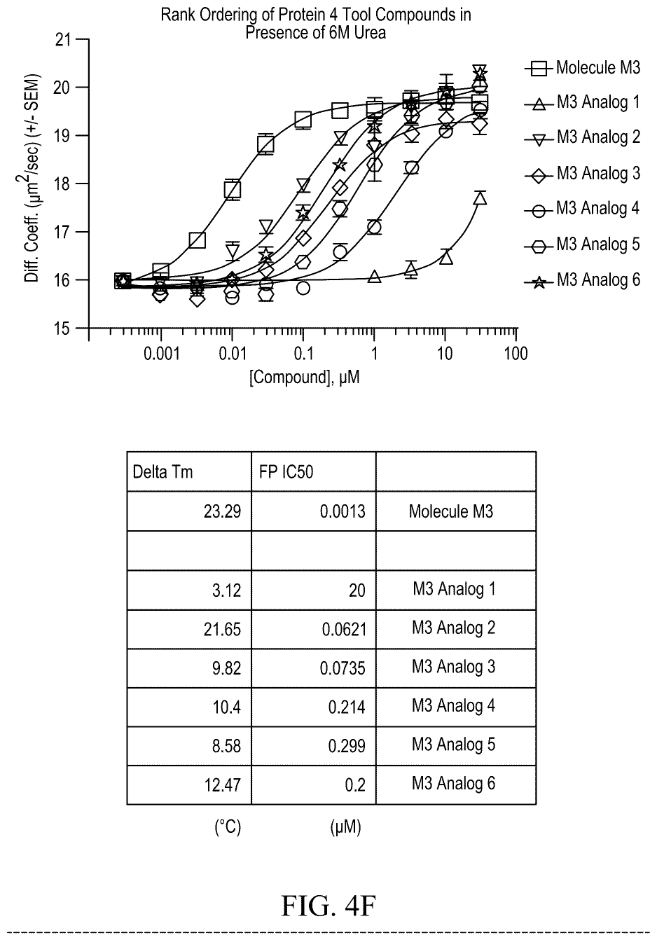

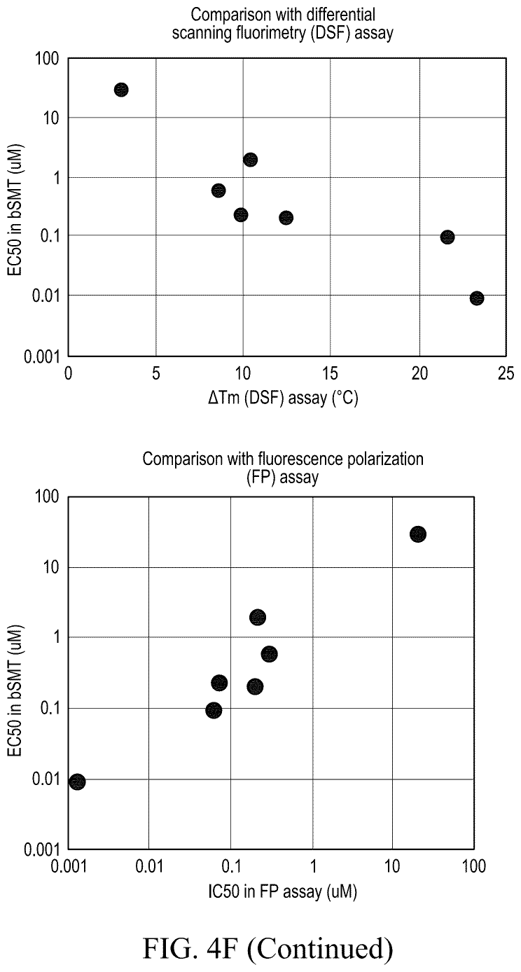

Labeled proteins were diluted to 4-200 pM in a buffer containing (25 mM HEPES, pH 7.9, 100 mM KCl, 12.5 mM MgCl₂, 0.1 mM EDTA, 1 mM DTT, 0.01% NP40, 0.3 mg/mL BSA) with glycerol supplemented to specific concentrations, and other additives such as chaotropic agents as specified. Small molecule compounds, i.e., organic molecules of less than 1 KDa, dissolved in DMSO were added to reaction based on DMSO tolerability of each assayed protein. 30 μL of each reaction was aliquoted onto 384-well glass-bottom CellVis plate for imaging under single-molecule imaging microscope. Protein 1 peptide used to disrupt a Protein 2-Protein 1 interaction was custom synthesized (Elim Biopharm) and supplemented as specified. Molecule MX used with Protein 3 was custom synthesized (CPC Scientific Inc.) and supplemented as specified. Pre-incubation before imaging may amplify the changes caused by the presence of chaotropic agents and small molecule binders. These incubation steps were inserted to specified experiments: 37° C. 1 hr followed by 4° C. 3 days (FIG. 4D), 37° C. 2 hr (FIG. 4E, middle panel), 4° C. 3 days followed by 37° C. 1 hr (FIG. 4E, bottom panel), and room temperature (25° C.) 2 hr (FIG. 4F).

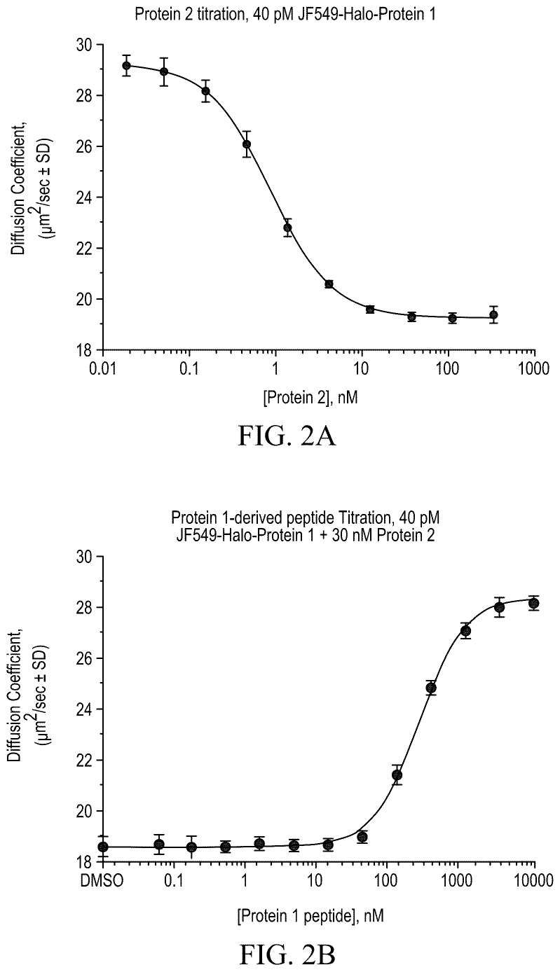

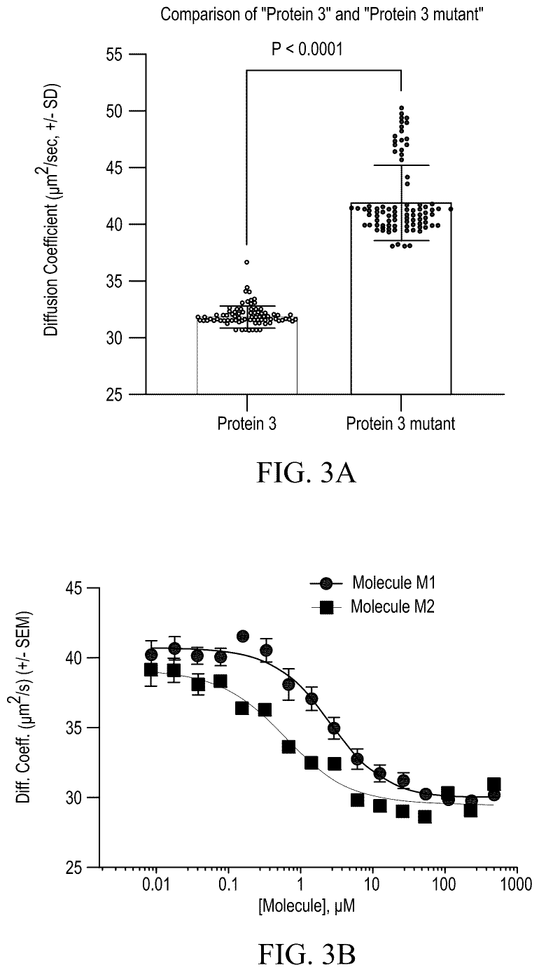

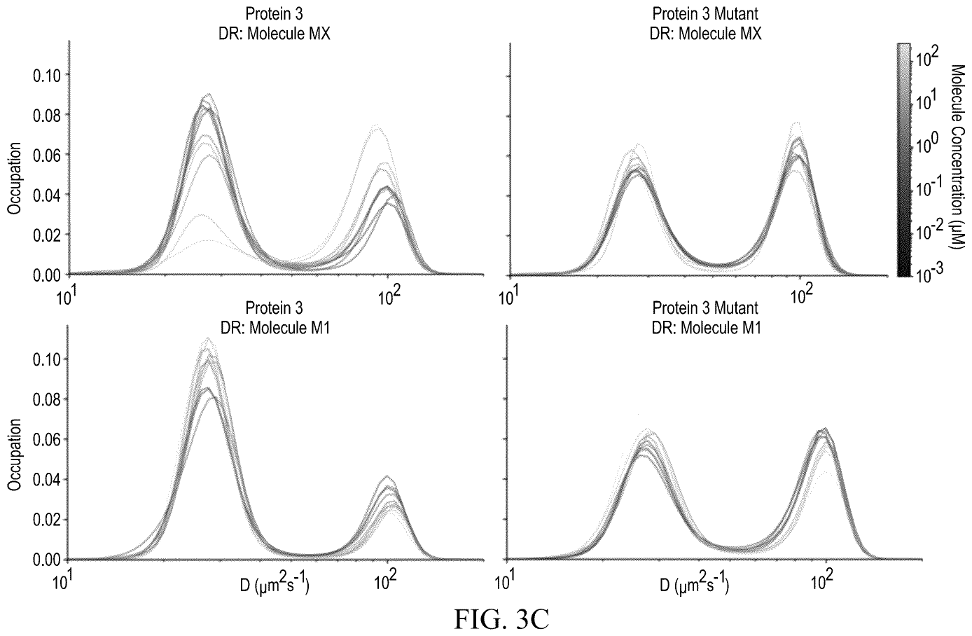

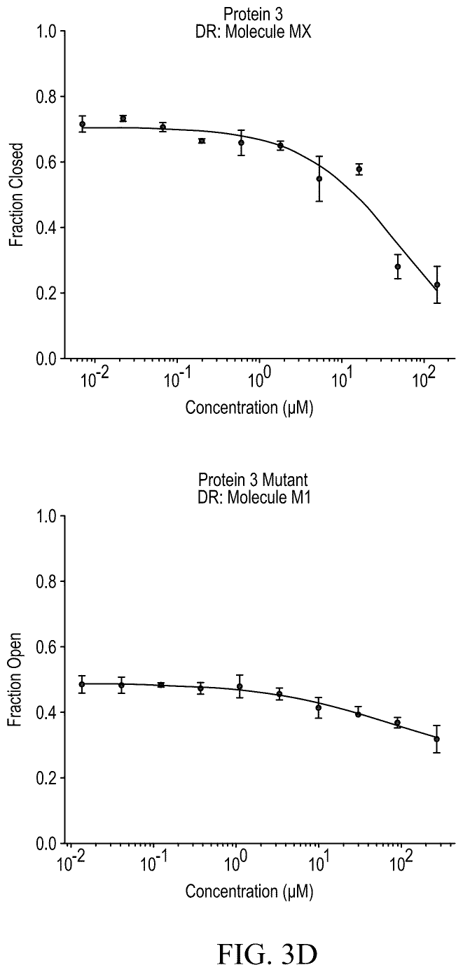

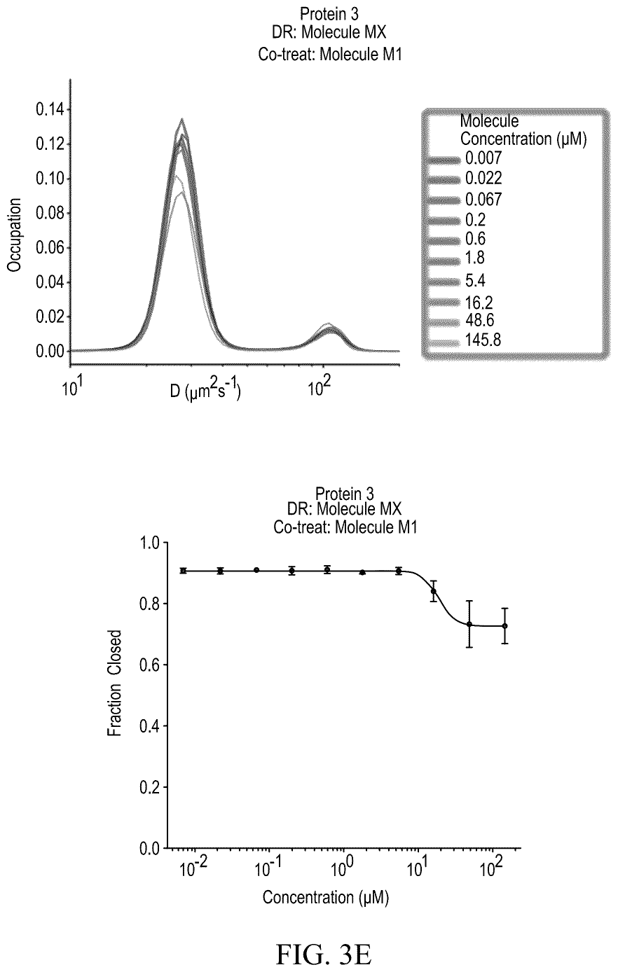

Single molecule tracking (SMT) images were acquired on an Oblique Line Scanning (OLS) microscope at 200 Hz (FIGS. 2A-3B and FIGS. 4B-6D) or 800 Hz (FIGS. 3C-3E). Exemplary OLS microscopes are described in PCT/US2023/085589. The data were processed with a custom pipeline operating on image sequences produced by the microscope. Briefly, individual emitters were detected by applying a generalized log-likelihood ratio test to every 11×11 subwindow in the image as described above (See Signal to noise ratio definition and quantification section below). Emitters were detected by identifying pixels with a log likelihood ratio exceeding 16. Detected emitters were localized to subpixel precision in a two-stage procedure. First, the subpixel location was estimated by computing points of maximum radial symmetry. Second, this estimate was used to seed an iterative Levenberg-Marquardt fitting routine to a 2D integrated Gaussian within a 11×11 pixel subwindow centered on the detection.

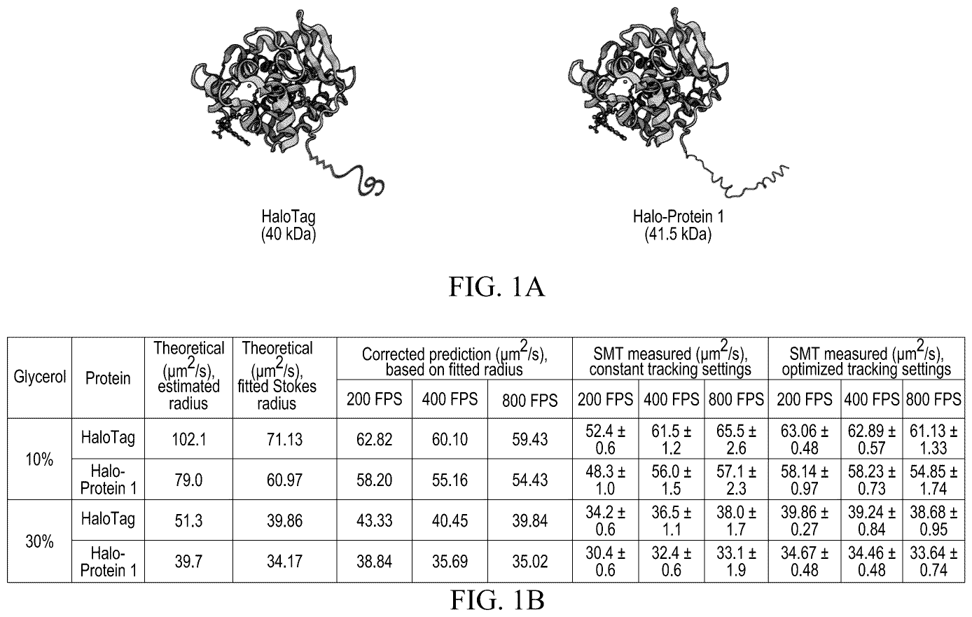

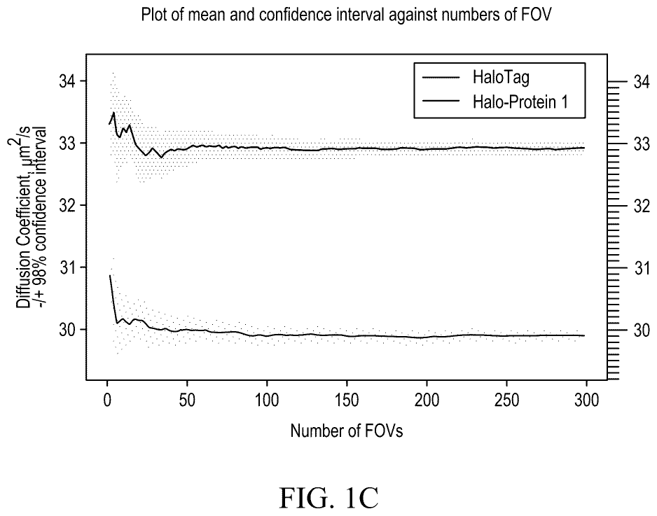

Localized emitters were linked in time to produce spatiotemporal trajectories using a modification of Sbalzerini's hill-climbing algorithm that uses Gibbs sampling to estimate data association uncertainty. In SMT, links longer than 2.5 μm (FIGS. 2A-3B and FIGS. 4B-6D) or 1.5 μm (FIGS. 3C-3E) and links over more than 2 gap frames were prohibited to limit association error.

### Signal to Noise Ratio Definition and Quantification:

Signal to noise ratio (SNR) is defined based on the likelihood ratio for a hypothesis test comparing: a target-absent condition, where the local image is modeled by the sum of a constant offset, and independent Gaussian-distributed noise; and a target-present condition, where the local image is modeled by the sum of a centrally located Gaussian peak (with known width but unknown amplitude), independent Gaussian-distributed noise, and a constant offset. The SNR is expressed as:

$$SNR = - \frac{{w}_{s}^{2}}{2} ln [ 1 - \frac{{( A {h}_{G} )}^{2}}{( {A}^{2} {h}_{u} ) - {( A {h}_{u} )}^{2} / {w}_{s}^{2}} ]$$

where:
A is the image, cropped to the current region of interest (ROI);
wₛ is the side length, in pixels, of the square ROI;
[symbol] is the inner product operator;
h_{G} is a zero-mean detection kernel, matched to the expected Gaussian target profile, and with

$$\sum {h}_{G}^{2} = 1$$

where the summation is taken over the ROI;
hᵤ is a uniform kernel (i.e., it has a value of 1 over the entire ROI).

### Differential Scanning Fluorimetry (DSF):

Unlabeled Protein 4 was diluted to 3 μM in buffer (25 mM HEPES, pH 7.4, 150 mM NaCl), with DMSO supplemented to 0.5% alone (vector control) or with tested compounds to 50 μM. Melting temperature (Tm) was measured with Prometheus Panta NanoDSF equipment and data processed with associated software.

### Fluorescence Polarization (FP) Assay:

20 nM of Protein 4 was mixed with 5 nM FAM labeled Protein 6 peptide (which binds to the same site of protein 4 as Molecule M3, with C-terminus capped with amine, Elim Biopharm) in a buffer containing (10 mM HEPES pH 7.5, 150 mM NaCl, 50 mM EDTA, 0.005% Tween 20, 0.1 mg/ml BSA and 1 mM DTT). Compounds were added with 0.2% DMSO. Signals were measured in 384-well plates with EnVision 2013 plate reader (Perkin Elmer). Half maximal inhibitory concentration (IC50) was determined using Prism software (GraphPad).

### Measurements on Spatiotemporal Trajectories:

When reporting the number of spatiotemporal trajectories, singlets (spatiotemporal trajectories with 1 detection) were excluded, as these do not contribute information to most dynamical estimates.

Average diffusion coefficients were computed with the mean squared displacement method (Dₑₛₜ=MSD_{2D}/4Δf). This estimator is expected to overestimate the diffusion coefficient by σ_{loc}²/Δt, where σ_{loc}² is the variance of the 1D localization error and Δt is the frame interval.

To resolve spatiotemporal trajectories in multiple dynamical states, the coefficients of a Brownian mixture model over a grid of diffusion coefficient values and localization error values were inferred using state arrays, a variational Bayesian routine based on the Dirichlet process mixture. Mixture components were selected as the Cartesian product of 100 diffusion coefficients log-spaced between 0.01 and 100 μm²/s and 31 localization error values from 0.02 to 0.08 μm (1D standard deviations). Occupations are reported as the mean posterior probabilities of each diffusion coefficient marginalized over all values of localization error. To make inference tractable, inference was limited to 10000 spatiotemporal trajectories randomly sampled from each well.

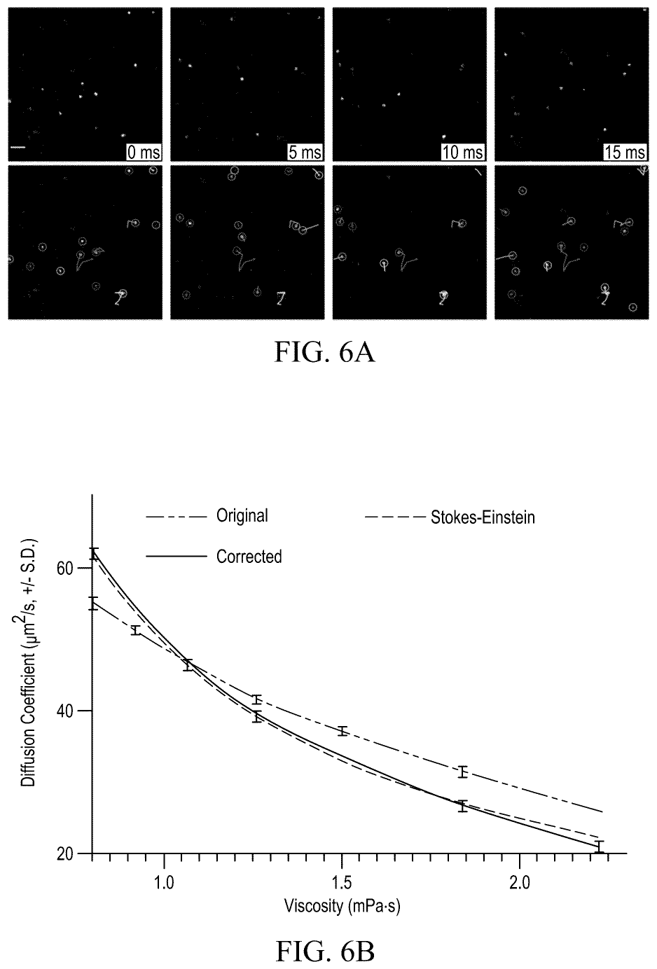

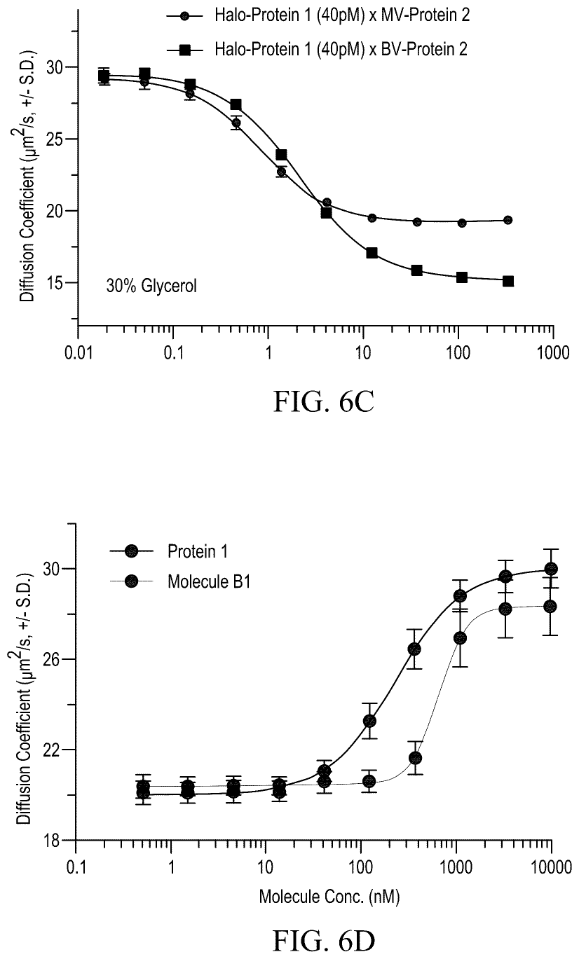

Bias estimation in single population samples was performed using analytical calculations that capture the probability of false linking and jump-length-distribution truncation due to a finite search radius. Spatial locations are uniformly distributed, allowing the probability of false linking to be estimated based on the observed emitter concentration and a nearest-neighbor linking model. This probabilistic analysis also allows characterization of the false-link jump length distribution. Single-population bias estimates incorporate these false-linking artifacts, and the prohibition on jump-length observations greater than the search radius. As shown in FIG. 6A, these bias expressions are used to improve diffusion-coefficient estimation accuracy.

### Empirical Estimate of Linking Precision:

To estimate the accuracy of the linking algorithm, a bootstrapping procedure was used. Detections from the first and second halves of the movie were superimposed, and the tracking algorithm was run on the resulting set of detections while blinded to the origin of each detection. From this the fraction of links generated where individual detections were joined from different halves of the movie were computed. Since this fraction neither accounts for erroneous links between detections in the same half of the movie nor for the effects of photobleaching, it forms a lower bound on the linking error rate (ERLB).

5.1. Example 1: SMT can Distinguish Proteins with Subtle Differences by Movement

This example shows the use of a method of the present disclosure for distinguishing between target proteins by analyzing the movement of the target proteins. This example further shows that a method of the present disclosure can be used to measure the diffusive properties of purified proteins in solution.

As described by the Stokes-Einstein equation, movement of a particle is dependent on the temperature, viscosity of the media and effective hydrated radius of the particle. For example, the size, shape, surface chemical/physical features and interaction with surrounding media can all contribute and affect the effective hydrated radius, which can in turn affect the movement of target molecules under certain conditions (e.g., temperature and media). Therefore, tracking the movement of particles can provide valuable information about their structural and functional properties.

To validate that the SMT system can reliably measure the movement of proteins in solution, the diffusion of JF₅₄₉-labeled HisHalo was measured with increasing concentrations of glycerol to modulate viscosity. His-Halo was diluted to 50 pM in imaging buffer consisting of 25 mM HEPES pH 7.4, 150 mM NaCl, 1 mM DTT, 0.03% (w/v) BSA, 0.01% NP-40, with glycerol levels spanning 0 to 40%. Dilutions were transferred to a 384 well-plate, which was incubated at 37° C. for 15 minutes prior to imaging using OLS. 22-44 well replicates were captured per concentration and acquired 4 FOVs per well at 200 frames per second, and the glycerol titration experiment was performed on two plates using the same protein preparation. Viscosity calculations were performed. As shown in FIG. 6A and FIG. 6B, the diffusion coefficient was estimated from tracking results and corrected using analytical expressions for tracking biases, resulting in a close match to the theoretical values predicted by the Stokes-Einstein equation.

Further experiments were performed to confirm that the SMT system can be used to distinguish between proteins. HaloTag and a HaloTag fused to a peptide from Protein 1 (referred to herein as “Halo-Protein 1”) were expressed and purified (FIG. 1A). The diffusion of both the HaloTag and Halo-Protein 1 were measured at 37° C. in the presence of different glycerol concentrations.

To estimate the expected diffusion coefficient, the hydration radius of HaloTag and Halo-Protein 1 were determined in an aqueous solution (50 mM HEPES, pH 7.5, 150 mM NaCl) using dynamic light scattering (DLS) and 2.39 nm and 3.09 nm was determined for HaloTag and Halo-Protein 1, respectively. This value is largely in agreement based on the estimation of HaloTag using a public resource (52.14.70.9/Run_hullrad.html), which gives a hydration radius of 2.14 nm for the core domain of the HaloTag protein. Without being limited to a particular theory, the slight difference is likely due to the presence of a tail of unstructured sequence in the purified protein used in this experiment. Another resource (www.met.reading.ac.uk/˜sws04cdw/viscosity_calc.html) was used to estimate the dynamic viscosity of glycerol as 0.00093 N*s/m² and 0.00185 N*s/m², for 10% v/v and 30% v/v glycerol, respectively. With the Boltz constant of 1.38E⁻²³, at a temperature of 37° C., the diffusion coefficient of HaloTag is expected to be 102.1 μm²/s and 51.3 μm²/s in 10% and 30% glycerol, respectively.

As shown in FIG. 1B, when HaloTag was measured under the SMT conditions at 200 frame per second (FPS), the diffusion coefficients for HaloTag at 10% and 30% glycerol were determined to be 52.4 and 34.2 μm²/s, respectively, which is overall slower than expected (FIG. 1B, column “Theoretical (μm²/s), estimated radius”). Increasing frame rates, however, results in measurements that mirror more closely the theoretical expectations. Similar results were observed with Halo-Protein 1 (FIG. 1B, column “SMT measured (μm²/s), constant tracking tr settings”). Remaining discrepancies between SMT measurements and theoretical values can be attributed to two sources. First, better measurements of diffusion coefficients were obtained when tracking settings (e.g., search radius) were optimized for each frame rate (FIG. 1B, last column). Second, the Stokes radius or hydration radius of a protein will depend strongly on the medium. The measured diffusion coefficients can be used with optimized tracking settings to fit an estimated Stokes radius in our experimental conditions. The Stokes radius of HaloTag was estimated to be 3.6 nm and that of HaloTag-Protein1 to be 4.2 nm. Using these radii gives the theoretical values in FIG. 1B, column “Theoretical (μm²/s), fitted Stokes radius”. Finally, the correction applied in FIG. 6B, which is based on a comprehensive tracking model that accounts for jump truncation, tracking errors and other effects, can be used to estimate the diffusion coefficients we will measure in our assay, given our estimated Stokes radii (FIG. 1B, “Corrected prediction (μm²/s), based on fitted radius”). The values in the “Corrected prediction” column and the “SMT measured, optimized tracking settings” column agree very well.).

FIG. 1C provides a plot of the average diffusion coefficient-/+98% confidence intervals of HaloTag and Halo-Protein 1 against the number of fields of view (FOVs) used for the calculation, as a guideline on data collection. Interestingly, despite the close similarity of these two proteins, the SMT technology can distinguish them under each of the tested conditions. This points to small changes in amino acid sequence of a protein having an effect on its movement (e.g., due to changing its hydration radius and interaction with surrounding molecules) significant enough that SMT is capable of detecting these kinds of otherwise subtle changes. In addition, despite the intrinsic difficulty in generating spatiotemporal trajectories for fast-diffusing protein in solution, measurements using the presently disclosed methods were validated by showing a strong overall agreement between experimental measurements and the corresponding Stokes-Einstein theory.

### 5.2. Example 2: SMT can Identify Protein-Protein Interactions

This example illustrates the use of a method of the present disclosure for identifying interactions between a protein and its ligand.

A direct application of the SMT methods disclosed herein is to detect protein-protein interactions, e.g., if a labeled protein binds to a partner of a significant size, the movement of the labeled protein should decrease significantly. It has been well documented that the N-terminus of Protein 1 binds to Protein 2 at high affinity. To determine if SMT can be used to identify protein-protein interaction, the interaction of Halo-Protein 1 (41.5 kD) with recombinant Protein 2 (85.6 kD) was analyzed.

GST-Protein 2 (dimer Protein 2) was made via bacterial expression as described above. The GST tag was removed via thrombin cleavage and purified by SEC to obtain monomer Protein 2. His-Halo-Protein 1 (1-53) was made via bacterial expression as described above, then labeled with JF549 and purified by SEC. Final concentration was determined via Nanodrop and gel band intensity imaged on Cy3 channel of ImageQuant800.

Halo-Protein 1 and Protein 2 were diluted in imaging buffer (25 mM HEPES-K pH=7.6, 0.1 M EDTA pH=8.0, 12.5 mM magnesium chloride, 100 mM potassium chloride, 0.2 mM PMSF, 1 mM DTT, 0.3 mg/ml BSA, 0.01% NP40, and 30% glycerol, pH=7.9) to final concentrations of 40 pM and 30 nM, respectively. Compounds were printed onto a 384 Cell Vis glass bottom plate via Echo 655, then incubated with Protein 2 for 10 minutes at room temperature prior to addition of Halo-Protein 1. Protein solutions were dispensed using an Integra VIAFLO. Plates were then incubated at 37° C. for 2 hours prior to imaging.

It was found that in the presence of 30% glycerol, titration of Protein 2 gradually slowed down the diffusion of Halo-Protein 1 (from ˜29.2 μm²/s to ˜19.4 μm²/s, measured at 40 pM) (FIG. 2A and FIG. 6C). As shown in FIG. 2A and FIG. 6C, a concentration-dependent decrease in Halo-Protein 1 diffusion with increasing concentrations of monovalent Protein 2 was observed. The apparent kD of 0.9 nM is largely in agreement with previous reports given the difference in buffer composition and measurement temperature. In addition, when a bivalent version of Protein 2 was titrated in, Halo-Protein 1 diffusion was shifted to a minimum of 15.08 μm²/s versus 19.39 μm²/s when monovalent Protein 2 was added. Additionally, when a free unlabeled Protein 1 peptide was titrated to the mixture of 40 pM Halo-Protein 1 and 30 nM Protein 2, the diffusion of Halo-Protein 1 was reversed in a dose dependent manner, suggesting competitive disruption (FIG. 2B and FIG. 6D).

To demonstrate application of the presently disclosed method in the context of drug discovery, a small-molecule inhibitor and a Protein 1 competing peptide were tested for their ability to alter the protein motion of JF₅₄₉-labeled Protein 1 in complex with Protein 2. A known inhibitor of Protein 2, Molecule B1, was able to increase the diffusion of JF₅₄₉-labeled Protein 1 in a dose-dependent response with a maximum effect similar to the competitive peptide (FIG. 6D).

As shown by these data, SMT measurement of protein diffusion can detect protein-protein interactions and protein-ligand interactions as well as inhibition of such interactions by competitive ligands in solution with potential sensitivity into the picomolar range.

The relevance of the disclosed method was demonstrated by capturing the interaction between two proteins, Protein 1 and Protein 2. The ability to leverage the disclosed method was also demonstrated by monitoring the disruption of this protein interaction with a competitive peptide and small molecule inhibitors, suggesting that an application of the disclosed method can be drug discovery. The presently disclosed method requires picomolar concentrations of purified protein enabling assay development of difficult to purify proteins and the sensitivity to measure sub-nanomolar affinities. It is hypothesized that ligand interactions can alter protein diffusion in ways beyond those caused by disruption of protein-protein interactions, including ligand-induced confirmational changes, protein stability changes and perhaps more subtle changes to protein hydration shell resulting in small but measurable changes in diffusion.

### 5.3. Example 3: Detection of Protein Conformation Changes

Many enzymes are regulated allosterically, which refers to binding of an effector to a site other than the enzyme's active site(s), with the effector regulating the enzymes' function by inducing structural/conformational changes. Such changes are likely to affect diffusion by changing the overall shape and surface features (chemical properties and topology) of proteins, which in turn affect their effective hydration radius and interaction with surrounding solution molecules. To test this feasibility, Protein 3 can be used as a model. Protein 3 is a phosphatase that is known to go through conformational changes through its activity cycles. Under resting conditions, it's largely in a more closed, inactive state. Upon binding to activating peptides, it resumes a more open and active conformation. Cancer cells often exploit this mechanism to gain proliferative advantages, by mutating critical amino residues that regulating this process. One example is Protein 3 mutant, which leads to predominantly an open and active conformation.

Because a fluorescent tag can, in certain instances, contribute to the overall movement characteristics of targeted proteins, multiple ways of protein labeling can be exploited. HaloTag method is a convenient self-catalyzed process but it requires the addition of a relatively large module of about 40 kD. As an alternative, SortaseTagging requires a much smaller attachment which is only ˜6 amino acid residues as an end product, that allows conjugation of fluorophore-attached small peptide by the enzyme sortase. These two methods provide flexibilities in the size and shape of the final labeled proteins for the convenience of SMT. The diffusion of SortaseTagged Protein 3 and Protein 3 mutant were compared using SMT.

Significant differences in their ensemble average diffusion were observed, with Protein 3 being around 32 μm²/s while the Protein 3 mutant being ˜42 μm²/s (FIG. 3A). When state arrays are used to estimate the occupancy of these proteins in different diffusive states, it is observed that both Protein 3 and Protein 3 mutant exist in 2 states, one with a peak diffusion coefficient around 30 μm²/s and another around 100 μm²/s (FIG. 3C). Protein 3 exists predominantly in the 30 μm²/s state, whereas the mutant exhibits more comparable occupancy in the 2 states but slightly more in the 100 μm²/s state. It is noted that faster states are consistently under-sampled in SMT, so Protein 3 mutant likely exists predominantly in the faster state, but by state arrays, occupancy in the two states looks more equivalent than they actually are.

This leads to the ensemble average diffusion coefficient of Protein 3 being around 32 μm²/s, while the mutant's ensemble average diffusion coefficient is higher (42 μm²/s). This also leads to the hypothesis that the state with a peak diffusion coefficient around 30 μm²/s represents the closed state, while the state with a peak diffusion coefficient around 100 μm²/s represents the open state, and that both Protein 3 and Protein 3 mutant exist as an equilibrium between these two states.

The effective hydration radius of Protein 3 and Protein 3 mutant have been previously reported to be 2.62 and 2.92 nm, respectively, which would suggest that the Protein 3 mutant should diffuse about 10% slower based on radius alone. Because the opposite trend was observed, there are other factors, beyond the pure hydration radius, that are contributing to the diffusion behavior of these proteins, which can be further exploited to understand their structure and function.

To confirm that the diffusion changes caused by mutation is a direct reflection of conformational changes, chemical compounds (Molecule M1 and Molecule M2) were titrated in that have been reported to switch the Protein 3 mutant back to the wild type-like conformation. A reversion in a dose dependent manner, indeed, was observed at the population level (FIG. 3B), and the rank order of the two compounds tested agrees with literature reports. This experiment demonstrated the effective use of SMT to detect organic molecules of less than 1 KDa binding to a protein as well as an exploration of the structural consequences.

To further confirm that conformational changes in Protein 3 can be detected, and that the two populations observed by state arrays represent the two conformations that Protein 3 is known to adopt, the effects of Molecule M1 and another molecule, Molecule MX, were observed by state array (FIG. 3C). Molecule MX is a binding-pocket interactor (rather than an allosteric interactor like Molecule M1) and should bias Protein 3 and Protein 3 mutant towards a conformation more like that of Protein 3 mutant's dominant conformation (i.e., the open conformation). In a dose-dependent manner, Molecule MX does indeed shift the occupancy of Protein 3 from predominantly the slower state to predominantly the faster state, consistent with the slower state representing Protein 3's main (closed) conformation, and the faster state representing Protein 3 mutant's main (open) state. Similarly, in a dose-dependent manner, Molecule M1 shifts the occupancy of Protein 3 mutant more towards the slower state, consistent with its known activity of biasing Protein 3 mutant towards a conformation more like that of Protein 3. The reverse is not true—Molecule MX has minimal effect on Protein 3 mutant's relative state occupancies, and Molecule M1 actually biases Protein 3 even further to the slower state.

State arrays can provide a superior method for quantifying relative occupancies in each conformation in dose titration experiments. Computing “fraction closed” (for Protein 3) or “fraction open” (for Protein 3 mutant) as a function of Molecule MX or M1 concentration respectively can give a bigger signal window than looking at ensemble-average diffusion coefficients (FIG. 3D compared to FIG. 3B). Here, the fractional occupancy in each state is determined by taking the average of the 10 to 50 μm²/s (for closed) or 50 to 200 μm²/s (for open) state array bins, weighted by each bin's occupancy, over the total weighted average diffusion coefficient over 10-200 μm²/s. Errors are standard errors over replicates.

Finally, it is demonstrated that this technology can be used to identify molecules that distinguish between these two conformational states. By state array analysis, in the presence of a constant concentration of Molecule M1 (which should bias Protein 3 to the slower state), it takes a higher concentration of Molecule MX to shift the occupancy of Protein 3 to the faster state than when Molecule MX is added alone (FIG. 3E). This indicates that Molecule M1 is selectively stabilizing the slower state. It is noted that Molecule M1 and Molecule MX bind to different sites (allosteric vs binding pocket), so this is not a competition effect. Consistent with this, even at the highest concentrations of Molecule MX in the presence of Molecule M1, the fraction of the closed state for Protein 3 plateaus at 0.7, much higher than the 0.2 fraction closed at the highest dose in the absence of Molecule M1. Since the effect of Molecule M1 cannot be completely reversed, Molecule MX is not competing Molecule M1 off.

5.4. Example 4: Detecting Binding to Proteins with Rigid Structures

This example illustrates the use of a method of the present disclosure for analyzing the interactions of a protein with an organic molecule of less than 1 KDa.

Many protein-ligand interactions do not lead to significant changes in three-dimension (3D) structures. While traditional organic molecule drugs generally fall in the range of 250 Da to 1 KDa, this would only amount to a few percent or less of mass of many proteins. Given the limited overall addition in mass by the binding of such a ligand to a protein, the effect of such binding on the hydration shell of the protein could be minimum, and therefore it could be challenging to detect this category of interactions using SMT. As an example, recombinantly expressed Protein 4, which has a well characterized organic molecule binder Molecule M3, was chosen for analysis. Unlike Protein 3 mutant, Protein 4 binding to its organic molecule ligand barely caused any changes to the 3D structure of the protein (FIG. 4A). A SortaseTag was attached to the protein and labeled with a JF549 fluorophore, and its diffusion was tracked either in the presence of DMSO (vector control) or Molecule M3. The presence of Molecule M3 did not cause any significant changes to the protein's diffusion (FIG. 4B).

In search for buffer conditions that might allow the detection of an organic molecule of less than 1 KDa binding to Protein 4 by diffusion measurement, chaotropic agents that are known to destabilize protein structures such as acetone, acetonitrile, DMSO, guanidinium and urea were analyzed (FIGS. 4C-4D). Without being bound by theory, it is hypothesized that chaotropic agents may partially unfold proteins, leading to one or more intermediate or unfolded states, and that the presence of an organic molecule binder could potentially stabilize one structure over others. Given that these structures would be expected to have different effective hydrated radius, they would be expected to lead to differentiated diffusion. As shown (FIGS. 4C-4D), multiple of the chaotropic agents tested, under multiple conditions, increased the difference between DMSO control (without Molecule M3) and Molecule M3 treated samples, with acetone, acetonitrile, urea and guanidinium being more significant than DMSO.

To analyze the impact of various experimental conditions, concentrations of chaotropic agents were titrated across a range of concentrations. Similar to the observations illustrated in FIGS. 4C-4D, when urea, guanidinium, and acetone were titrated in FIG. 4E, a general trend in reduction of diffusion was also observed. Without being limited to a particular theory, an explanation is that protein unfolding leads to increased effective hydrated radius, thereby reducing diffusion. In addition, at certain concentrations, urea and guanidinium provided conditions distinguishing the diffusion of DMSO and Molecule M3 treated Protein 4 proteins with larger dynamic windows, more specifically urea at 6-7 M and guanidinium at around 1 M (FIG. 4E). The overall trend is that Molecule M3 leads to increased diffusion, consistent with the hypothesis that it stabilizes protein structure and reduces unfolding. Unlike the smooth transition with urea, titration of guanidinium resulted in a substantial drop in the diffusion of DMSO treated protein at around 1 M. The complexity of this titration curve implies that additional factors are affecting the diffusion of tested proteins, and by modulating buffer composition new insights concerning the diffusion states of the tested proteins are possible.

To test whether this method is suitable for structure-activity-relationship (SAR) analysis, a dose titration of a series of Molecule M3 analogs to Protein 4 in the presence of 6 M urea was performed (FIG. 4F). A gradual increase in diffusion of Protein 4 was observed upon the addition of these compounds, and apparent EC50 were determined to compare their potencies. These compounds have also been characterized using other conventional assays such as fluorescence polarization (FP, measuring the replacement of a fluorescently labeled probe binding to the same pocket) and differential scanning fluorimetry (DSF, measuring the melting/unfolding of protein) assays commonly used for SAR analysis. The rank order of potency measured by SMT is largely in agreement with FP, and the more potent molecules as measured by SMT generally exhibited larger increases in melting temperature (Tm) as measured by DSF. The results indicate that SMT is indeed suitable for use in SAR analysis.

### 5.5. Example 5: Tracking Diffusion of an Intrinsically Disordered Region

This example illustrates the use of a method of the present disclosure for analyzing the interactions of a protein comprising an intrinsically disordered region (IDR) with other molecules, e.g., organic molecules of less than 1 KDa.

The human proteome comprises a continuum of proteins/domains from the structured to the unstructured. IDRs are abundant within this continuum and play crucial roles in biology. Due to the lack of stable structures, however, IDRs are not amenable to conventional biophysical approaches, e.g., crystallography and electron microscopy. In contrast to other proteins/domains existing with relatively stable tertiary structures, IDRs generally exist as ensembles of interchangeable conformations. Because these conformational changes can lead to changes in the protein's effective hydrated radius, IDR-containing proteins are amenable to analyses focusing on movement and tracking such movement can provide insights on an IDR's conformational profile, including how it is related to the protein's biological function.

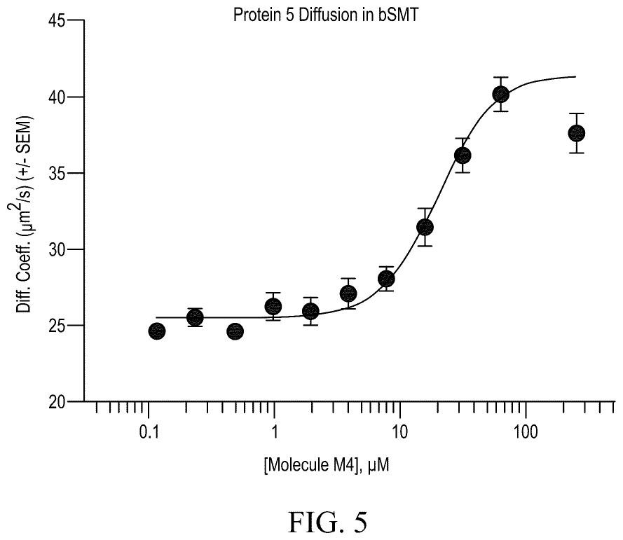

Protein 5 was analyzed as a model of IDR, which has been reported to be largely void of secondary structure. The movement of the Protein 5 labeled with JF549 via a SortaseTag at the N-terminus was tracked and its diffusion coefficient was determined. Molecule M4 is a covalent modifier reported to bind to Protein 5. When the compound was titrated in, a gradual increase in Protein 5 diffusion was observed (FIG. 5). Without being limited to a particular theory, these results point to a change in the interaction between Protein 5 and buffer components, e.g., the direct interaction between the protein and Molecule M4. Therefore, SMT provides an entirely new tool to study IDRs and their interaction with ligands.

### 5.6. Example 6: Single Molecule Fluorescence Polarization

This example illustrates the use of single molecule fluorescence polarization to exploit the orientation-dependent fluorescence properties of fluorophores sufficiently rigidly attached to target biomolecules, thereby enabling the characterization of the latter's molecular dynamics. This approach, complemented by translational single-molecule tracking (SMT), provides insights into molecule size, conformational changes, and binding dynamics across diverse microenvironments. While translational SMT is, in addition to molecule size, influenced by factors like local conditions such as viscosity, matrix-like or cellular boundaries, and microfluidic flows, single molecule fluorescence polarization, in its initial approximation, operates independently of the molecule's surroundings. Single molecule fluorescence polarization can capture temporal dynamics through spatiotemporal correlation functions and offers valuable information about molecule size and changes due to binding and conformational events.

### Methodology:

Several methodologies can be used for performing single molecule fluorescence polarization: (1) Time-Correlated Single-Photon Counting (TCSPC), (2) step-scan pump-probe techniques, (3) ultra-long lifetime fluorophores, and (4) advanced optical techniques.

Time-Correlated Single-Photon Counting (TCSPC), by detecting individual photon arrival times with picosecond accuracy, allows for the precise measurement of fluorescence lifetimes, which can be important for resolving dynamics on the nanosecond scale. TCSPC uses 2D TCSPC via Single Photon Avalanche Diode (SPAD) arrays and photon-counting modules to achieve extremely high temporal resolution. This method paired with polarization-sensitive readouts is ideal for studying fast rotational dynamics and short fluorescence lifetimes typical of many biological fluorophores. It enhances the ability to discriminate between different fluorophore states or orientations.

Step-scan pump-probe techniques modulate the polarization of excitation and probing light and the resulting fluorescence emission over time provides insights into the orientation/rotation and dynamics of fluorophores. Step-scan pump-probe techniques use step-scan pump-probe spectromicroscopy to measure polarization-dependent and time-correlated fluorescence depletion dynamics. It is useful for optically filtering subpopulations of a distinct molecular size, orientation, or dynamic, as well as for studying dynamics in systems where fluorophore orientation changes rapidly post-excitation, such as in protein dynamics or structural changes in membranes.

Ultra-long lifetime fluorophores exhibit fluorescence lifetimes much longer than the typical fluorescence decay times of organic fluorophores (e.g., nanoseconds to microseconds or longer). Ultra-long lifetime fluorophores (e.g., carbon dots, quantum dots, nanodiamonds) can be used to extend depolarization times significantly beyond typical fluorescence lifetimes. Ultra-long lifetime fluorophores can be used to measure rotational dynamics and depolarization processes over extended timescales, enabling the study of slow molecular motions or environments with restricted mobility.

Advanced optical techniques, e.g., stress-engineered optics, enable precise spatial manipulation and control of light polarization states, which are important for techniques such as wobble anisotropy and polarization filtering. Advanced optical techniques include advanced optical methodologies involving phase and polarization control. These techniques achieve high-precision polarization-dependent measurements, allowing extraction of detailed information about fluorophore orientation and dynamics.

### DISCUSSION

At the single-molecule and single-photon levels, single molecule fluorescence polarization utilizes fluorescence characteristics governed by the alignment of a fluorophore's transition dipole moment. For common fluorophores such as rhodamine-based JF549, absorption and emission probabilities correlate with the fluorophore's orientation relative to incident light polarization, known as fluorescence anisotropy. This phenomenon, influenced by the molecule's rotational dynamics within its excited state lifetime, provides a temporal contrast that reveals molecular size, inter- and intramolecular dynamics, and binding events.

Current camera-based translational SMT faces challenges due to its integration time (>20 μs), significantly longer than typical fluorescence lifetimes (<100 ns) and rotational dynamics (<100 ns). To address this, the temporally-averaged fluorescence intensity dependency at specific polarizations (i.e., wobble anisotropy) can be measured using the methodology described herein.

In the context of oligomerization, single molecule fluorescence polarization provides contrast by detecting changes in fluorophore orientation and rotational freedom upon oligomer formation. This contrast arises from alterations in the anisotropic properties of fluorescence emission as monomers transition to oligomeric states.

Additionally, single molecule fluorescence polarization reveals how conformational changes affect the orientation and dynamics of fluorophores attached to specific sites within a molecule. This capability enables the characterization of dynamic structural transitions, such as protein folding-unfolding events or nucleic acid conformational changes, with exquisite sensitivity and spatial resolution.

Key methodologies include time-resolved anisotropy measurements, which track changes in fluorescence polarization over time, revealing the kinetics of oligomer formation, dissociation, and intramolecular conformational dynamics. Polarization-sensitive imaging techniques, coupled with advanced optical controls, facilitate the spatial mapping of these events within complex biological environments.

Leveraging single molecule fluorescence polarization enables the characterization of both oligomerization processes and intramolecular conformational dynamics at a level of detail inaccessible to ensemble averaging methods. This approach not only provides insights into the stoichiometry and structural dynamics of biomolecules but also offers a means to correlate these dynamics with functional changes in biological systems. Ultimately, single molecule fluorescence polarization can unravel the complexities of molecular assembly pathways and dynamic structural changes, and advance our understanding of biological function and disease mechanisms.

### 6. EXEMPLARY EMBODIMENTS

A. The present disclosure provides a method of identifying an interaction between a target molecule and a test molecule comprising: (a) contacting a cell-free sample comprising a plurality of the target molecule with a plurality of the test molecule; (b) tracking a plurality of the target molecules over time to provide a plurality of spatiotemporal trajectories and/or measurements of rotational motion; (c) analyzing the plurality of spatiotemporal trajectories and/or measurements of rotational motion to determine the target molecule's movement in the presence of the test molecule; and (d) comparing the target molecule's movement obtained in (c) with a reference target molecule's movement, wherein the reference target molecule's movement is the movement of the target molecule in the absence of the test molecule; wherein a change in the target molecule's movement compared to the reference target molecule's movement indicates an interaction between the target molecule and the test molecule.
A1. The method of A, wherein the method is a high throughput method.
A2. The method of A or A1, wherein the change in movement is calculated as a change in: (a) diffusion coefficient obtained from a maximum likelihood estimator; (b) geometric mean posterior diffusion coefficient; (c) median of the jump length distribution; (d) 3rd quartile of the jump length distribution; (e) median radius of gyration; (f) mean posterior diffusion coefficient; (g) mean squared displacement; (h) median bond angle; (i) spatiotemporal trajectory length; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.
A3. The method of any one of A-A2, wherein the change in movement is calculated as a change in diffusion coefficient.
A4. The method of any one of A-A3, wherein an increase or decrease in the target molecule's movement compared to the reference target molecule's movement indicates an interaction between the target molecule and the test molecule.
A5. The method of any one of A-A4, wherein the target molecule is an organic molecule less than 1 KDa.
A6. The method of any one of A-A5, wherein the target molecule is selected from the group consisting of a peptide, a protein, a nucleic acid, a carbohydrate, and a lipid.
A7. The method of A6, wherein the protein is selected from the group consisting of an antibody, a receptor, and an enzyme.
A8. The method of A6 or A7, wherein the protein comprises a disordered domain.
A9. The method of A8, wherein the protein does not comprise a structured domain.
A10. The method of A6, wherein the peptide is a ligand.
A11. The method of any one of A-A4, wherein the target molecule is a nanomaterial.
A12. The method of any one of A-A4, wherein the target molecule is a synthetic polymer.
A13. The method of any one of A-A12, wherein the test molecule is an organic molecule less than 1 KDa.
A14. The method of any one of A-A13, wherein the test molecule is selected from the group consisting of a peptide, a protein, a nucleic acid, a carbohydrate, and a lipid.
A15. The method of A14, wherein the protein is selected from the group consisting of an antibody, a receptor, and an enzyme.
A16. The method of A14 or A15, wherein the protein comprises a disordered domain.
A17. The method of A16, wherein the protein does not comprise a structured domain.
A18. The method of A14, wherein the peptide is a ligand.
A19. The method of any one of A-A12, wherein the test molecule is a nanomaterial.
A20. The method of any one of A-A12, wherein the test molecule is a synthetic polymer.
A21. The method of any one of A-A20, wherein the target molecule is labeled.
A22. The method of any one of A-A21, wherein the target molecule is fluorescently labeled.
A23. The method of any one of A-A22, wherein the test molecule is labeled.
A24. The method of any one of A-A23, wherein the test molecule is fluorescently labeled.
A25. The method of any one of A-A24, wherein the change in the target molecule's movement compared to the reference target molecule's movement indicates a direct or indirect interaction between the target molecule and the test molecule.
A26. The method of any one of A-A25, wherein the interaction between the target molecule and the test molecule results in a conformational change in the target molecule.
A27. The method of any one of A-A25, wherein the interaction between the target molecule and the test molecule results in an increase in the mass of the target molecule.
A28. The method of any one of A-A27, wherein the cell-free sample comprises a solution.
A29. The method of A28, wherein the solution comprises a chaotropic agent, a carrier, a viscosity agent, or a combination thereof.
A30. The method of A29, wherein the solution comprises a viscosity agent.
A31. The method of A30, wherein the viscosity agent comprises glycerol.
A32. The method of A31, wherein the solution comprises at least about 30% glycerol.
A33. The method of any one of A28-A32, wherein the solution comprises a temperature gradient, a chemical gradient, at least two phases, or a combination thereof.
A34. The method of any one of A-A33, wherein the cell-free sample has a volume of about 0.1 μl to about 100 μl.
A35. The method of any one of A-A34, wherein the change in movement is measured as a change in the diffusion coefficient of the target molecule as compared to the reference target molecule's diffusion coefficient, and the change is at least about 0.001%, at least about 0.005%, at least 0.01%, at least about 0.05%, at least about 0.1%, at least about 0.5%, at least about 1%, at least about 1.5%, at least about 2% or at least about 3%, at least about 4%, at least about 5%, at least about 6%, at least about 7%, at least about 8%, at least about 9% or at least about 10%.
A36. The method of any one of A-A35, wherein the duration of the target molecule's movement change compared to the duration of the reference target molecule's movement change indicates an interaction between the target molecule and the test molecule.
A37. The method of any one of A-A36, wherein the reversibility of the target molecule's movement change compared to the reversibility of the reference target molecule's movement change indicates an interaction between the target molecule and the test molecule.
A38. The method of any one of A-A37, wherein the interaction between the target molecule and the test molecule results in an oligomerization of the target molecule.
A39. The method of any one of A-A38, wherein the interaction between the target molecule and the test molecule is reversible.
A40. The method of any one of A-A39, wherein the interaction between the target molecule and the test molecule is irreversible.
A41. The method of any one of A-A40, wherein the target molecule and/or test molecule is a component of a mixture composed of a bacterial extract, cell extract, tissue extract, plant extract, or animal extract.
A42. The method of A41, wherein the bacterial extract, cell extract, tissue extract, plant extract, or animal extract is a lysate.
A43. The method of any one of A-A42, wherein the target molecule and/or test molecule is a component of a mixture composed of serum, blood, and other biological samples.
A44. The method of any one of A-A43, wherein the target molecule and/or test molecule is a component of a mixture composed of buffers and glycerol.
A45. The method of any one of A-A44, wherein the target molecule and/or the test molecule is labeled with a fluorescent protein.
A46. The method of any one of A-A45, wherein the target molecule and/or the test molecule is labeled by conjugation of a synthetic nanomaterial or polymer.
A47. The method of any one of A-A46, wherein the target molecule and/or the test molecule is labeled with a fluorescent protein.
A48. The method of any one of A-A47, wherein the plurality of target molecules are conjugated to a plurality of fluorescent labels.
A49. The method of any one of claims A-A48, wherein the target molecule and/or the test molecule is labeled by conjugation to a synthetic nanomaterial or polymer.
A50. The method of any one of A-A49, wherein the interaction between the target molecule and the test molecule results in a change in the temperature stability of the target molecule.
B. The present disclosure provides a method for identifying an interaction between a test molecule and a target protein comprising a disordered domain comprising: (a) contacting a cell-free sample comprising a plurality of the target protein with a plurality of the test molecule; (b) tracking a plurality of the target protein over time to provide a plurality of spatiotemporal trajectories and/or measurements of rotational motion; (c) analyzing the plurality of spatiotemporal trajectories and/or measurements of rotational motion to determine the target protein's movement in the presence of the test molecule; and (d) comparing the target protein's movement obtained in (c) with a reference target protein's movement, wherein the reference target protein's movement is the movement of the target protein in the absence of the test molecule; wherein a change in the movement of the target protein comprising the disordered domain compared to the reference target protein's movement indicates an interaction between the target protein comprising the disordered domain and the test molecule.
B1. The method of B, wherein the method is a high throughput method.
B2. The method of B or B1, wherein the change in movement is calculated as a change in: (a) diffusion coefficient obtained from a maximum likelihood estimator; (b) geometric mean posterior diffusion coefficient; (c) median of the jump length distribution; (d) 3rd quartile of the jump length distribution; (e) median radius of gyration; (f) mean posterior diffusion coefficient; (g) mean squared displacement; (h) median bond angle; (i) spatiotemporal trajectory length; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.
B3. The method of any one of B-B2, wherein the change in movement is calculated as a change in diffusion coefficient.
B4. The method of any one of B-B3, wherein an increase or decrease in the movement of the target protein comprising the disordered domain compared to the reference target protein's movement indicates an interaction between the target protein comprising the disordered domain and the test molecule.
B5. The method of any one of B-B4, wherein the target protein comprising the disordered domain is selected from the group consisting of an antibody, a receptor, and an enzyme.
B6. The method of any one of B-B5, wherein the target protein comprising the disordered domain does not comprise a structured domain.
B7. The method of any one of B-B6, wherein the test molecule is an organic molecule less than 1 KDa.
B8. The method of any one of B-B7, wherein the test molecule is selected from the group consisting of a peptide, a protein, a nucleic acid, a carbohydrate, and a lipid.
B9. The method of B8, wherein the protein is selected from the group consisting of an antibody, a receptor, and an enzyme.
B10. The method of B8 or B9, wherein the protein comprises a disordered domain.
B11. The method of B10, wherein the protein does not comprise a structured domain.
B12. The method of B8, wherein the peptide is a ligand.
B13. The method of any one of B-B12, wherein the target protein comprising the disordered domain is labeled.
B14. The method of any one of B-B13, wherein the target protein comprising the disordered domain is fluorescently labeled.
B15. The method of any one of B-B14, wherein the test molecule is labeled.
B16. The method of any one of B-B15, wherein the test molecule is fluorescently labeled.
B17. The method of any one of B-B16, wherein the change in the movement of the target protein comprising the disordered domain compared to the reference target protein's movement indicates a direct or indirect interaction between the target protein comprising the disordered domain and the test molecule.
B18. The method of any one of B-B17, wherein the interaction between the target protein comprising the disordered domain and the test molecule results in a conformational change in the target protein.
B19. The method of any one of B-B17, wherein the interaction between the target protein comprising the disordered domain and the test molecule results in an increase in the mass of the target protein.
B20. The method of any one of B-B19, wherein the cell-free sample comprises a solution.
B21. The method of B20, wherein the solution comprises a viscosity agent.
B22. The method of B20 or B21, wherein the solution comprises a chaotropic agent, a carrier, a viscosity agent, or a combination thereof.
B23. The method of B22, wherein the viscosity agent comprises glycerol.
B24. The method of B23, wherein the solution comprises at least about 30% glycerol.
B25. The method of any one of B20-B24, wherein the solution comprises a temperature gradient, a chemical gradient, at least two phases, or a combination thereof.
B26. The method of any one of B-B25, wherein the cell-free sample has a volume of about 0.1 μl to about 100 μl.
B27. The method of any one of B-B26, wherein the change in movement is measured as a change in the diffusion coefficient of the target protein as compared to the reference target protein's diffusion coefficient, and the change is at least about 0.001%, at least about 0.005%, at least 0.01%, at least about 0.05%, at least about 0.1%, at least about 0.5%, at least about 1%, at least about 1.5%, at least about 2% or at least about 3%, at least about 4%, at least about 5%, at least about 6%, at least about 7%, at least about 8%, at least about 9% or at least about 10%.
B28. The method of any one of B-B27, wherein the duration of the movement change of the target protein comprising the disorder domain compared to the duration of the reference target protein's movement change indicates an interaction between the target protein and the test molecule.
B29. The method of any one of B-B28, wherein the reversibility of the movement change of the target protein comprising the disorder domain compared to the reversibility of the reference target protein's movement change indicates an interaction between the target protein and the test molecule.
B30. The method of any one of B-B29, wherein the interaction between the target protein comprising the disordered domain and test molecule results in an oligomerization of the target protein.
B31. The method of any one of B-B30, wherein the interaction between the target protein comprising the disordered domain and the test molecule is reversible.
B32. The method of any one of B-B31, wherein the interaction between the target protein comprising the disordered domain and the test molecule is irreversible.
B33. The method of any one of B-B32, wherein the target protein comprising the disordered domain and/or test molecule is a component of a mixture composed of a bacterial extract, cell extract, tissue extract, plant extract, or animal extract.
B34. The method of B33, wherein the bacterial extract, cell extract, tissue extract, plant extract, or animal extract is a lysate.
B35. The method of any one of B-B34, wherein the target protein comprising the disordered domain and/or test molecule is a component of a mixture composed of serum, blood, and other biological samples.
B36. The method of any one of B-B35, wherein the target protein comprising the disordered domain and/or test molecule is a component of a mixture composed of buffers and glycerol.
B37. The method of any one of B-B36, wherein the target protein and/or the test molecule is labeled with a fluorescent protein.
B38. The method of any one of B-B37, wherein the target protein and/or the test molecule is labeled by conjugation of a synthetic nanomaterial or polymer.
B39. The method of any one of B-B38, wherein the conformational change in the target protein results in a change in the temperature stability of the target protein.
C. The present disclosure provides a method for analyzing a target molecule in a test solution comprising: (a) tracking a plurality of the target molecules over time in the test solution to provide a plurality of spatiotemporal trajectories and/or measurements of rotational motion; (b) analyzing the plurality of spatiotemporal trajectories and/or measurements of rotational motion to determine the target molecule's movement in the test solution; and (c) comparing the target molecule's movement obtained in (b) with a reference target molecule's movement, wherein the reference target molecule's movement is the movement of the target molecule in a reference solution; wherein: (i) a change in the target molecule's movement compared to the reference target molecule's movement indicates that the target molecule is affected by the test solution; or (ii) no change in the target molecule's movement compared to the reference target molecule's movement indicates that the target molecule is not affected by the test solution.
C1. The method of C, wherein the method is a high throughput method.
C2. The method of C or C1, wherein the change in movement is calculated as a change in: (a) diffusion coefficient obtained from a maximum likelihood estimator; (b) geometric mean posterior diffusion coefficient; (c) median of the jump length distribution; (d) 3rd quartile of the jump length distribution; (e) median radius of gyration; (f) mean posterior diffusion coefficient; (g) mean squared displacement; (g) median bond angle; (i) spatiotemporal trajectory length; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.
C3. The method of any one of C-C2, wherein the change in movement is calculated as a change in diffusion coefficient.
C4. The method of any one of C-C3, wherein the target molecule is an organic molecule less than 1 KDa.
C5. The method of any one of C-C4, wherein the target molecule is selected from the group consisting of a peptide, a protein, a nucleic acid, a carbohydrate, and a lipid.
C6. The method of C5, wherein the protein is selected from the group consisting of an antibody, a receptor, and an enzyme.
C7. The method of C5 or C6, wherein the protein comprises a disordered domain.
C8. The method of C7, wherein the protein does not comprise a structured domain.
C9. The method of C5, wherein the peptide is a ligand.
C10. The method of any one of C-C3, wherein the target molecule is a nanomaterial.
C11. The method of any one of C-C3, wherein the target molecule is a synthetic polymer.
C12. The method of any one of C-C11, wherein the target molecule is labeled.
C13. The method of any one of C-C12, wherein the target molecule is fluorescently labeled.
C14. The method of any one of C-C13, wherein the test solution comprises a chaotropic agent.
C15. The method of C14, wherein the chaotropic agent is urea.
C16. The method of any one of C-C15, wherein the test solution comprises a viscosity agent.
C17. The method of C16, wherein the viscosity agent is glycerol.
C18. The method of any one of C-C17, wherein the test solution comprises a gradient.
C19. The method of C17, wherein the gradient is a temperature gradient, a chemical gradient, or a combination thereof.
C20. The method of any one of C-C19, wherein the test solution comprises at least two phases, or a combination thereof.
C21. The method of any one of C-C20, wherein the interaction between the target protein and the test solution results in a conformational change in the target molecule.
C22. The method of any one of C-C21, wherein the duration of the target molecule's movement change compared to the duration of the reference target molecule's movement change indicates that the target molecule is affected by the test solution.
C23. The method of any one of C-C22, wherein the reversibility of the target molecule's movement change compared to the reversibility of the reference target molecule's movement change indicates that the target molecule is affected by the test solution.
C24. The method of any one of C-C23, wherein the interaction between the target molecule and the test solution is reversible.
C25. The method of any one of C-C24, wherein the interaction between the target molecule and the test solution is irreversible.
C26. The method of any one of C-C25, wherein the target molecule and/or reference target molecule is a component of a mixture composed of a bacterial extract, cell extract, tissue extract, plant extract, or animal extract.
C27. The method of C26, wherein the bacterial extract, cell extract, tissue extract, plant extract, or animal extract is a lysate.
C28. The method of any one of C-C27, wherein the target molecule and/or reference target molecule is a component of a mixture composed of serum, blood, and other biological samples.
C29. The method of any one of C-C28, wherein the target molecule and/or reference target molecule is a component of a mixture comprising one or more buffers and glycerol.
C30. The method of any one of C-C29, wherein the target molecule and/or reference target molecule is labeled with a fluorescent protein.
C31. The method of any one of C-C30, wherein the target molecule and/or reference target molecule is labeled by conjugation of a synthetic nanomaterial or polymer.
D. The present disclosure provides a method for determining the form of a target molecule comprising: (a) tracking a plurality of the target molecules over time in a cell-free sample to provide a plurality of spatiotemporal trajectories and/or measurements of rotational motion; (b) analyzing the plurality of spatiotemporal trajectories and/or measurements of rotational motion to determine the target molecule's movement; and (c) comparing the target molecule's movement obtained in (b) with a reference target molecule's movement, wherein the reference target molecule's movement is the movement of one form of the target molecule; wherein: (i) a change in the target molecule's movement compared to the reference target molecule's movement indicates that the target molecule is of a different form than the reference target molecule; or (ii) no change in the target molecule's movement compared to the reference target molecule's movement indicates that the target molecule is of the same form as the reference target molecule.
D1. The method of D, wherein the method is a high throughput method.
D2. The method of D or D1, wherein the change in movement is calculated as a change in: (a) diffusion coefficient obtained from a maximum likelihood estimator; (b) geometric mean posterior diffusion coefficient; (c) median of the jump length distribution; (c) 3rd quartile of the jump length distribution; (d) median radius of gyration; (e) mean posterior diffusion coefficient; (f) mean squared displacement; (h) median bond angle; (i) spatiotemporal trajectory length; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.
D3. The method of any one of D-D2, wherein the change in movement is calculated as a change in diffusion coefficient.
D4. The method of any one of D-D3, wherein the target molecule is an organic molecule less than 1 KDa.
D5. The method of any one of D-D4, wherein the target molecule is selected from the group consisting of a peptide, a protein, a nucleic acid, a carbohydrate, and a lipid.
D6. The method of D5, wherein the protein is selected from the group consisting of an antibody, a receptor, and an enzyme.
D7. The method of D5 or D6, wherein the protein comprises a disordered domain.
D8. The method of D7, wherein the protein does not comprise a structured domain.
D9. The method of D5, wherein the peptide is a ligand.
D10. The method of any one of D-D3, wherein the target molecule is a nanomaterial.
D11. The method of any one of D-D3, wherein the target molecule is a synthetic polymer.
D12. The method of any one of D-D11, wherein the target molecule is labeled.
D13. The method of any one of D-D12, wherein the target molecule is fluorescently labeled.
D14. The method of any one of D-D13, wherein the cell-free sample comprises a solution.
D15. The method of D14, wherein the solution comprises a chaotropic agent, a carrier, a viscosity agent, or a combination thereof.
D16. The method of D15, wherein the solution comprises a chaotropic agent.
D17. The method of D16, wherein the chaotropic agent is urea.
D18. The method of any one of D14-D17, wherein the solution comprises a viscosity agent.
D19. The method of D18, wherein the viscosity agent comprises glycerol.
D20. The method of D19, wherein the solution comprises at least about 30% glycerol.
D21. The method of any one of D14-D20, wherein the solution comprises a temperature gradient, a chemical gradient, at least two phases, or a combination thereof.
D22. The method of any one of D-D21, wherein the cell-free sample has a volume of about 0.1 μl to about 100 μl.
D23. The method of any one of D-D22, wherein the change in movement is measured as a change in the diffusion coefficient of the target molecule as compared to the reference target molecule's diffusion coefficient, and the change is at least about 0.001%, at least about 0.005%, at least 0.01%, at least about 0.05%, at least about 0.1%, at least about 0.5%, at least about 1%, at least about 1.5%, at least about 2% or at least about 3%, at least about 4%, at least about 5%, at least about 6%, at least about 7%, at least about 8%, at least about 9% or at least about 10%.
D24. The method of any one of D-D23, wherein the form of the target molecule is a post-translationally modified form of the target molecule.
D25. The method of any one of D-D23, wherein the form of the target molecule is a wild type form of the target molecule.
D26. The method of any one of D-D23, wherein the form of the target molecule is a mutant form of the target molecule.
D27. The method of any one of D-D26, wherein the duration of the target molecule's movement change compared to the duration of the reference target molecule's movement change indicates that the target molecule is of a different form than the reference target molecule.
D28. The method of any one of D-D27, wherein the reversibility of the target molecule's movement change compared to the reversibility of the reference target molecule's movement change indicates that the target molecule is of a different form than the reference target molecule.
D29. The method of any one of D-D28, wherein the target molecule and/or reference target molecule is a component of a mixture composed of a bacterial extract, cell extract, tissue extract, plant extract, or animal extract.
D30. The method of D29, wherein the bacterial extract, cell extract, tissue extract, plant extract, or animal extract is a lysate.
D31. The method of any one of D-D30, wherein the target molecule and/or reference target molecule is a component of a mixture composed of serum, blood, and other biological samples.
D32. The method of any one of D-D31, wherein the target molecule and/or reference target molecule is a component of a mixture composed of buffers and glycerol. D33. The method of any one of D-D32, wherein the target molecule and/or reference target molecule is labeled with a fluorescent protein.
D34. The method of any one of D-D33, wherein the target molecule and/or reference target molecule is labeled by conjugation of a synthetic nanomaterial or polymer.
E. The present disclosure provides a method for identifying a test molecule that can distinguish between at least two target molecules comprising: (a) contacting a cell-free sample comprising a plurality of a first target molecule with a plurality of the test molecule; (b) tracking a plurality of the first target molecules over time to provide a plurality of spatiotemporal trajectories and/or measurements of rotational motion; (c) analyzing the plurality of spatiotemporal trajectories and/or measurements of rotational motion to determine the first target molecule's movement in the presence of the test molecule; (d) contacting a cell-free sample comprising a plurality of a second target molecule with a plurality of the test molecule; (e) tracking a plurality of the second target molecules over time to provide a plurality of spatiotemporal trajectories and/or measurements of rotational motion; (f) analyzing the plurality of spatiotemporal trajectories and/or measurements of rotational motion to determine the second target molecule's movement in the presence of the test molecule; and (g) comparing the first and second target molecules' movement obtained in (c) and (f); wherein a change in the movement of the first and second target molecules indicates that the test molecule can distinguish between the two target molecules.
E1. The method of E, wherein the method is a high throughput method.
E2. The method of E or E1, wherein the change in movement is calculated as a change in: (a) diffusion coefficient obtained from a maximum likelihood estimator; (b) geometric mean posterior diffusion coefficient; (c) median of the jump length distribution; (d) 3rd quartile of the jump length distribution; (e) median radius of gyration; (f) mean posterior diffusion coefficient; (g) mean squared displacement; (h) median bond angle; (i) spatiotemporal trajectory length; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.
E3. The method of any one of E-E2, wherein the change in movement is calculated as a change in diffusion coefficient.
E4. The method of any one of E-E3, wherein the target molecule is an organic molecule less than 1 KDa.
E5. The method of any one of E-E4, wherein the target molecule is selected from the group consisting of a peptide, a protein, a nucleic acid, a carbohydrate, and a lipid.
E6. The method of E5, wherein the protein is selected from the group consisting of an antibody, a receptor, and an enzyme.
E7. The method of E5 or E6, wherein the protein comprises a disordered domain.
E8. The method of E7, wherein the protein does not comprise a structured domain.
E9. The method of E5, wherein the peptide is a ligand.
E10. The method of any one of E-E3, wherein the target molecule is a nanomaterial.
E11. The method of any one of E-E3, wherein the target molecule is a synthetic polymer.
E12. The method of any one of E-E11, wherein the test molecule is an organic molecule less than 1 KDa.
E13. The method of any one of E-E12, wherein the test molecule is selected from the group consisting of a peptide, a protein, a nucleic acid, a carbohydrate, and a lipid.
E14. The method of E13, wherein the protein is selected from the group consisting of an antibody, a receptor, and an enzyme.
E15. The method of E13 or E14, wherein the protein comprises a disordered domain.
E16. The method of E15, wherein the protein does not comprise a structured domain.
E17. The method of E13, wherein the peptide is a ligand.
E18. The method of any one of E-E17, wherein the target molecule is labeled.
E19. The method of any one of E-E18, wherein the target molecule is fluorescently labeled.
E20. The method of any one of E-E19, wherein the test molecule is labeled.
E21. The method of any one of E-E20, wherein the test molecule is fluorescently labeled.
E22. The method of any one of E-E21, wherein the first target molecule and the second target molecule are related target molecules.
E23. The method of E22, wherein at least one of the target molecules is a wild type form of the target molecule.
E24. The method of E22, wherein at least one of the target molecules is a mutant form of the target molecule.
E25. The method of E22, wherein the first target molecule and the second target molecule are the same or different conformational states of the target molecule.
E26. The method of E22, wherein at least one of the target molecules is a post-translationally modified form of the target molecule.
E27. The method of E22, wherein the first target molecule and the second target molecule are homologs, orthologs or paralogs.
E28. The method of any one of E-E21, wherein the first target molecule and the second target molecule are unrelated target molecules.
E29. The method of any one of E-E28, further comprising: contacting a cell-free sample comprising a plurality of a third target molecule with a plurality of the test molecule; tracking a plurality of the third target molecules over time to provide a third plurality of spatiotemporal trajectories and/or measurements of rotational motion; analyzing the third plurality of spatiotemporal trajectories and/or measurements of rotational motion to determine the movement of the third target molecules in the presence of the test molecule; and comparing the third target molecule's movement to the movements obtained in (c) and (f); wherein a change in the movement of the third target molecule relative to the first and second target molecules indicates that the test molecule can distinguish between the three target molecules.
E30. The method of any one of E-E29, wherein the duration of the movement change of the first and second target molecules indicates that the test molecule can distinguish between the two target molecules.
E31. The method of any one of E-E30, wherein the reversibility of the movement change of the first and second target molecules indicates that the test molecule can distinguish between the two target molecules.
E32. The method of any one of E-E31, wherein the first target molecule, second target molecule and/or test molecule is a component of a mixture composed of a bacterial extract, cell extract, tissue extract, plant extract, or animal extract.
E33. The method of E32, wherein the bacterial extract, cell extract, tissue extract, plant extract, or animal extract is a lysate.
E34. The method of any one of E-E33, wherein the first target molecule, second target molecule, and/or test molecule is a component of a mixture composed of serum, blood, and other biological samples.
E35. The method of any one of E-E34, wherein the first target molecule, second target molecule, and/or test molecule is a component of a mixture composed of buffers and glycerol.
E36. The method of any one of E-E35, wherein the first target molecule, second target molecule and/or test molecule is labeled with a fluorescent protein.
E37. The method of any one of E-E36, wherein the first target molecule, second target molecule and/or test molecule is labeled by conjugation of a synthetic nanomaterial or polymer.
E38. The method of any one of E-E37, wherein the first and second target molecules are distinguished by a difference in their fluorescent labels.
F. The present disclosure provides a method for identifying one or more test molecules that induce a conformational change in a target molecule comprising: (a) contacting cell-free samples comprising a plurality of the target molecule with a plurality of test molecules, where each sample is contacted with a different test molecule; (b) tracking a plurality of the target molecules in each sample over time to provide a plurality of spatiotemporal trajectories and/or measurements of rotational motion; (c) analyzing the plurality of spatiotemporal trajectories and/or measurements of rotational motion to determine the target molecule's movement in the presence of each of the different test molecules; and (d) comparing the target molecule's movement obtained in (c) with a reference target molecule's movement, wherein the reference target molecule's movement is the movement of the target molecule in the presence of a test molecule that does not induce a conformational change in the target molecule; wherein a change in the target molecule's movement compared to the reference target molecule's movement indicates an interaction between the target molecule and the one or more test molecules that induce a conformational change in the target molecule.
F1. The method of F, wherein the method is a high throughput method.
F2. The method of F or F1, wherein the change in movement is calculated as a change in: (a) diffusion coefficient obtained from a maximum likelihood estimator; (b) geometric mean posterior diffusion coefficient; (c) the median of the jump length distribution; (b) 3rd quartile of the jump length distribution; (c) median radius of gyration; (d) mean posterior diffusion coefficient; (f) mean squared displacement; (g) median bond angle; (i) the spatiotemporal trajectory length; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.
F3. The method of any one of F-F2, wherein the change in movement is calculated as a change in diffusion coefficient.
F4. The method of any one of F-F3, wherein the target molecule is an organic molecule less than 1 KDa.
F5. The method of any one of F-F3, wherein the target molecule is selected from the group consisting of a peptide, a protein, a nucleic acid, a carbohydrate, and a lipid.
F6. The method of F5, wherein the protein is selected from the group consisting of an antibody, a receptor, and an enzyme.
F7. The method of F5 or F6, wherein the protein comprises a disordered domain.
F8. The method of F7, wherein the protein does not comprise a structured domain.
F9. The method of F5, wherein the peptide is a ligand.
F10. The method of any one of F-F3, wherein the target molecule is a nanomaterial.
F11. The method of any one of F-F3, wherein the target molecule is a synthetic polymer.
F12. The method of any one of F-F11, wherein the test molecule is an organic molecule less than about 1 KDa.
F13. The method of any one of F-F12, wherein the test molecule is selected from the group consisting of a peptide, a protein, a nucleic acid, a carbohydrate, and a lipid.
F14. The method of F13, wherein the protein is selected from the group consisting of an antibody, a receptor, and an enzyme.
F15. The method of F13 or F14, wherein the protein comprises a disordered domain.
F16. The method of F15, wherein the protein does not comprise a structured domain.
F17. The method of F13, wherein the peptide is a ligand.
F18. The method of any one of F-F17, wherein the target molecule is labeled.
F19. The method of any one of F-F18, wherein the target molecule is fluorescently labeled.
F20. The method of any one of F-F19, wherein the test molecule is labeled.
F21. The method of any one of F-F20, wherein the test molecule is fluorescently labeled.
F22. The method of any one of F-F21, wherein the cell-free sample comprises a solution.
F23. The method of F22, wherein the solution comprises a chaotropic agent, a viscosity agent, or a combination thereof.
F24. The method of F23, wherein the viscosity agent comprises glycerol.
F25. The method of F24, wherein the solution comprises at least about 30% glycerol.
F26. The method of any one of F22-F25, wherein the solution comprises a temperature gradient, a chemical gradient, at least two phases, or a combination thereof.
F27. The method of any one of F-F26, wherein the sample has a volume of about 0.1 μl to about 100 μl.
F28. The method of any one of F-F27, wherein the change in movement is measured as a change in the diffusion coefficient of the target molecule in the presence of a test molecule and in the presence of a reference test molecule that does not induce a confirmation change, and the change is at least about 0.001%, at least about 0.005%, at least 0.01%, at least about 0.05%, at least about 0.1%, at least about 0.5%, at least about 1%, at least about 1.5%, at least about 2% or at least about 3%, at least about 4%, at least about 5%, at least about 6%, at least about 7%, at least about 8%, at least about 9% or at least about 10%.
F29. The method of any one of F-F28, wherein the duration of the target molecule's movement change compared to the duration of the reference target molecule's movement change indicates an interaction between the target molecule and the one or more test molecules that induce a conformational change in the target molecule.
F30. The method of any one of F-F29, wherein the reversibility of the target molecule's movement change compared to the reversibility of the reference target molecule's movement change indicates an interaction between the target molecule and the one or more test molecules that induce a conformational change in the target molecule.
F31. The method of any one of F-F30, wherein the interaction between the target molecule and the one or more test molecules is reversible.
F32. The method of any one of F-F31, wherein the interaction between the target molecule and the one or more test molecules is irreversible.
F33. The method of any one of F-F32, wherein the target molecule, reference target molecule and/or one or more test molecules is a component of a mixture composed of a bacterial extract, cell extract, tissue extract, plant extract, or animal extract.
F34. The method of F33, wherein the bacterial extract, cell extract, tissue extract, plant extract, or animal extract is a lysate.
F35. The method of any one of F-F34, wherein the target molecule, reference target molecule and/or one or more test molecules is a component of a mixture composed of serum, blood, and other biological samples.
F36. The method of any one of F-F35, wherein the target molecule, reference target molecule and/or one or more test molecules is a component of a mixture composed of buffers and glycerol.
F37. The method of any one of F-F36, wherein the target molecule, reference target molecule and/or one or more test molecules is labeled with a fluorescent protein.
F38. The method of any one of F-F37, wherein the target molecule, reference target molecule and/or one or more test molecule is labeled by conjugation of a synthetic nanomaterial or polymer.
F39. The method of any one of F-F38, wherein the conformational change in the target molecule is associated with a change in the target molecule's temperature stability.
G. The present disclosure provides a method for determining a dose response for the interaction of a target molecule and a test molecule comprising: (a) contacting a plurality of cell-free samples comprising a plurality of the target molecule with the test molecule, where plurality of cell-free samples are contacted with a range of test molecule doses; (b) tracking a plurality of the target molecules over time in the presence of the range of doses of the test molecule to provide a plurality of spatiotemporal trajectories and/or measurements of rotational motion; (c) analyzing the plurality of spatiotemporal trajectories and/or measurements of rotational motion of the target molecules in the presence of the range of doses of the test molecule to determine the target molecule's movement at each dose of test molecule; (d) comparing the target molecule's movement obtained at different test molecule doses in step (c) to determine the dose response of the target molecule to the test molecule.
G1. The present disclosure provides a method for determining a difference in dose response to a test molecule by two target molecules comprising: (a) contacting a first plurality of cell-free samples comprising a plurality of the first target molecule with the test molecule, where first plurality of cell-free samples are contacted with a range of test molecule doses; (b) tracking a plurality of the target molecules over time in the presence of the range of doses of the test molecule to provide a first plurality of spatiotemporal trajectories and/or measurements of rotational motion; (c) analyzing the plurality of spatiotemporal trajectories and/or measurements of rotational motion of the target molecules in the presence of the range of doses of the test molecule to determine the first target molecule's movement at each concentration of test molecule; (d) comparing the target molecule's movement obtained at different test molecule doses in step (c) to determine the dose response of the target molecule to the test molecule; (e) repeating steps (a)-(d) with a second target molecule to determine the dose response of the second target molecule to the test molecule; and (f) comparing the dose response of the first target molecule to the dose response of the second target molecule to determine the difference in the response of the first and second target molecules to the test molecule.
G2. The method of G or G1, wherein the method is a high throughput method.
G3. The method of G or G1, wherein the movement is calculated as: (a) diffusion coefficient of the plurality of spatiotemporal trajectories obtained from a maximum likelihood estimator; (b) geometric mean posterior diffusion coefficient of the plurality of spatiotemporal trajectories; (c) the median of the jump length distribution of the plurality of spatiotemporal trajectories; (d) 3rd quartile of the jump length distribution of the plurality of spatiotemporal trajectories; (e) median radius of gyration of the plurality of spatiotemporal trajectories; (f) mean posterior diffusion coefficient of the plurality of spatiotemporal trajectories; (g) mean squared displacement of the plurality of spatiotemporal trajectories; (h) median bond angle of the plurality of spatiotemporal trajectories; (i) the spatiotemporal trajectory length of the plurality of spatiotemporal trajectories; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.
G4. The method of any one of G-G3, wherein the movement is calculated as a diffusion coefficient.
G5. The method of any one of G-G4, wherein the target molecule is an organic molecule less than 1 KDa.
G6. The method of any one of G-G5, wherein the target molecule is selected from the group consisting of a peptide, a protein, a nucleic acid, a carbohydrate, and a lipid.
G7. The method of G6, wherein the protein is selected from the group consisting of an antibody, a receptor, and an enzyme.
G8. The method of G6 or G7, wherein the protein comprises a disordered domain.
G9. The method of G8, wherein the protein does not comprise a structured domain.
G10. The method of G6, wherein the peptide is a ligand.
G11. The method of any one of G-G4, wherein the target molecule is a nanomaterial.
G12. The method of any one of G-G4, wherein the target molecule is a synthetic polymer.
G13. The method of any one of G-G12, wherein the test molecule is an organic molecule less than 1 KDa.
G14. The method of any one of G-G13, wherein the test molecule is selected from the group consisting of a peptide, a protein, a nucleic acid, a carbohydrate, and a lipid.
G15. The method of G14, wherein the protein is selected from the group consisting of an antibody, a receptor, and an enzyme.
G16. The method of G14 or G15, wherein the protein comprises a disordered domain.
G17. The method of G16, wherein the protein does not comprise a structured domain.
G18. The method of G14, wherein the peptide is a ligand.
G19. The method of any one of G-G18, wherein the target molecule is labeled.
G20. The method of any one of G-G19, wherein the target molecule is fluorescently labeled.
G21. The method of any one of G-G20, wherein the test molecule is labeled.
G22. The method of any one of G-G21, wherein the test molecule is fluorescently labeled.
G23. The method of any one of G1-G22, wherein the first target molecule and the second target molecule are related target molecules.
G24. The method of G23, wherein at least one of the target molecules is a wild type form of the target molecule.
G25. The method of G23, wherein at least one of the target molecules is a mutant form of the target molecule.
G26. The method of G23, wherein the first target molecule and the second target molecule are the same or different conformational states of the target molecule.
G27. The method of G23, wherein at least one of the target molecules is a post-translationally modified form of the target molecule.
G28. The method of G23, wherein the first target molecule and the second target molecule are homologs, orthologs or paralogs.
G29. The method of G1, wherein the first target molecule and the second target molecule are unrelated target molecules.
G30. The method of G1, wherein first and second cell-free samples comprises a solution that comprises a chaotropic agent a carrier, a viscosity agent, or a combination thereof.
G31. The method of G30, wherein the solution comprises a viscosity agent.
G32. The method of G31, wherein the viscosity agent comprises glycerol.
G33. The method of G32, wherein the solution comprises at least about 30% glycerol.
G34. The method of any one of G30-G33, wherein the solution comprises a temperature gradient, a chemical gradient, at least two phases, or a combination thereof.
G35. The method of any one of G-G34, wherein comparing the target molecule's movement obtained at different test molecule doses in step (c) further comprises comparing the duration of the target molecule's movement changes.
G36. The method of any one of G-G35, wherein comparing the target molecule's movement obtained at different test molecule doses in step (c) further comprises comparing the reversibility of the target molecule's movement changes.
G37. The method of any one of G-G36, wherein the interaction between the target molecule and test molecule is reversible.
G38. The method of any one of G-G37, wherein the interaction between the target molecule and test molecule is irreversible.
G39. The method of any one of G-G38, wherein the target molecule and/or test molecule is a component of a mixture composed of a bacterial extract, cell extract, tissue extract, plant extract, or animal extract.
G40. The method of G39, wherein the bacterial extract, cell extract, tissue extract, plant extract, or animal extract is a lysate.
G41. The method of any one of G-G40, wherein the target molecule and/or test molecule is a component of a mixture composed of serum, blood, and other biological samples.
G42. The method of any one of G-G41, wherein the target molecule and/or test molecule is a component of a mixture composed of buffers and glycerol.
G43. The method of any one of G-G42, wherein the target molecule and/or test molecule is labeled with a fluorescent protein.
G44. The method of any one of G-G43, wherein the target molecule and/or test molecule is labeled by conjugation of a synthetic nanomaterial or polymer.
G45. The method of any one of G-G44, wherein the first target molecule and the second target molecule are distinguished by a difference in their fluorescent labels.
G46. The method of any one of G-G45, wherein three or more target molecules are analyzed.
H. The present disclosure provides a method for identifying a test molecule that can distinguish between at least two target molecules comprising: (a) contacting a cell-free sample comprising a plurality of a first target molecule and a plurality of a second target molecule with a plurality of the test molecule; (b) tracking a plurality of the first target molecules over time to provide a plurality of spatiotemporal trajectories and/or measurements of rotational motion; (c) analyzing the plurality of spatiotemporal trajectories and/or measurements of rotational motion to determine the first target molecule's movement in the presence of the test molecule; (d) tracking a plurality of the second target molecules over time to provide a plurality of spatiotemporal trajectories and/or measurements of rotational motion; (e) analyzing the plurality of spatiotemporal trajectories and/or measurements of rotational motion to determine the second target molecule's movement in the presence of the test molecule; and (f) comparing the first and second target molecules' movement obtained in (c) and (f); wherein a change in the movement of the first and second target molecules indicates that the test molecule can distinguish between the two target molecules.
H1. The method of H, wherein the method is a high throughput method.
H2. The method of H or H1, wherein the change in movement is calculated as a change in: (a) diffusion coefficient obtained from a maximum likelihood estimator; (b) geometric mean posterior diffusion coefficient; (c) median of the jump length distribution; (d) 3rd quartile of the jump length distribution; (e) median radius of gyration; (f) mean posterior diffusion coefficient; (g) mean squared displacement; (h) median bond angle; (i) spatiotemporal trajectory length; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.
H3. The method of any one of H-H2, wherein the change in movement is calculated as a change in diffusion coefficient.
H4. The method of H-H3, wherein the target molecule is an organic molecule less than 1 KDa.
H5. The method of H-H4, wherein the target molecule is selected from the group consisting of a peptide, a protein, a nucleic acid, a carbohydrate, and a lipid.
H6. The method of H5, wherein the protein is selected from the group consisting of an antibody, a receptor, and an enzyme.
H7. The method of H5 or H6, wherein the protein comprises a disordered domain.
H8. The method of H7, wherein the protein does not comprise a structured domain.
H9. The method of H5, wherein the peptide is a ligand.
H10. The method of H-H3, wherein the target molecule is a nanomaterial.
H11. The method of H-H3, wherein the target molecule is a synthetic polymer.
H12. The method of any one of H-H11, wherein the test molecule is an organic molecule less than 1 KDa.
H13. The method of any one of H-H12, wherein the test molecule is selected from the group consisting of a peptide, a protein, a nucleic acid, a carbohydrate, and a lipid.
H14. The method of H13, wherein the protein is selected from the group consisting of an antibody, a receptor, and an enzyme.
H15. The method of H13 or H14, wherein the protein comprises a disordered domain.
H16. The method of H15, wherein the protein does not comprise a structured domain.
H17. The method of H13, wherein the peptide is a ligand.
H18. The method of any one of H-H17, wherein the target molecule is labeled.
H19. The method of any one of H-H18, wherein the target molecule is fluorescently labeled.
H20. The method of any one of H-H19, wherein the test molecule is labeled.
H21. The method of any one of H-H20, wherein the test molecule is fluorescently labeled.
H22. The method of any one of H-H21, wherein the first target molecule and the second target molecule are related target molecules.
H23. The method of H22, wherein at least one of the target molecules is a wild type form of the target molecule.
H24. The method of H22, wherein at least one of the target molecules is a mutant form of the target molecule.
H25. The method of H22, wherein the first target molecule and the second target molecule are the same or different conformational states of the target molecule.
H26. The method of H22, wherein at least one of the target molecules is a post-translationally modified form of the target molecule.
H27. The method of H22, wherein the first target molecule and the second target molecule are homologs, orthologs or paralogs.
H28. The method of any one of H-H21, wherein the first target molecule and the second target molecule are unrelated target molecules.
H29. The method of any one of H-H28, further comprising: contacting a cell-free sample comprising a plurality of a third target molecule with a plurality of the test molecule; tracking a plurality of the third target molecules over time to provide a third plurality of spatiotemporal trajectories and/or measurements of rotational motion; analyzing the third plurality of spatiotemporal trajectories and/or measurements of rotational motion to determine the movement of the third target molecules in the presence of the test molecule; and comparing the third target molecule's movement to the movements obtained in (c) and (f); wherein a change in the movement of the third target molecule relative to the first and second target molecules indicates that the test molecule can distinguish between the three target molecules.
H30. The method of any one of H-H29, wherein the duration of the first molecule's movement change compared to the duration of the second molecule's movement change indicates that the test molecule can distinguish between the two target molecules.
H31. The method of any one of H-H30, wherein the reversibility of the first molecule's movement change compared to the reversibility of the second molecule's movement change indicates that the test molecule can distinguish between the two target molecules.
H32. The method of any one of H-H31, wherein the interaction between the target molecule and test molecule is reversible.
H33. The method of any one of H-H32, wherein the interaction between the target molecule and test molecule is irreversible.
H34. The method of any one of H-H33, wherein the first target molecule, second target molecule and/or test molecule is a component of a mixture composed of a bacterial extract, cell extract, tissue extract, plant extract, or animal extract.
H35. The method of H34, wherein the bacterial extract, cell extract, tissue extract, plant extract, or animal extract is a lysate.
H36. The method of any one of H-H35, wherein the first target molecule, second target molecule, and/or test molecule is a component of a mixture composed of serum, blood, and other biological samples.
H37. The method of any one of H-H36, wherein the first target molecule, second target molecule, and/or test molecule is a component of a mixture composed of buffers and glycerol.
H38. The method of any one of H-H37, wherein the first target molecule, second target molecule and/or test molecule is labeled with a fluorescent protein.
H39. The method of any one of H-H38, wherein the first target molecule, second target molecule and/or test molecule is labeled by conjugation of a synthetic nanomaterial or polymer.
I. The present disclosure provides a method for analyzing a test solution comprising a target molecule comprising: (a) tracking a plurality of a target molecule over time in the test solution to provide a plurality of spatiotemporal trajectories and/or measurements of rotational motion; (b) analyzing the plurality of spatiotemporal trajectories and/or measurements of rotational motion to determine the target molecule's movement in the test solution; and (c) comparing the target molecule's movement obtained in (b) with a reference target molecule's movement, wherein the reference target molecule's movement is the movement of the target molecule in a reference solution; wherein: the comparing of step (c) is used to determine a property of the test solution.
I1. The method of I, wherein the method is a high throughput method.
I2. The method of I or I1, wherein the target molecule's movement is calculated as a: (a) diffusion coefficient obtained from a maximum likelihood estimator; (b) geometric mean posterior diffusion coefficient; (c) median of the jump length distribution; (d) 3rd quartile of the jump length distribution; (e) median radius of gyration; (f) mean posterior diffusion coefficient; (g) mean squared displacement; (g) median bond angle; (i) spatiotemporal trajectory length; (j) anisotropy decay time; (k) spatial extent of detection; (l) number and wavelength of conjugated fluorescent labels; (m) occupancy in different diffusive states obtained from state arrays; (n) polarization of conjugated fluorescent labels; or (o) state occupation via inference.
I3. The method of any one of I-I2, wherein the change in movement is calculated as a change in diffusion coefficient.
I4. The method of any one of I-I3, wherein the target molecule is an organic molecule less than 1 KDa.
I5. The method of any one of I-I4, wherein the target molecule is selected from the group consisting of a peptide, a protein, a nucleic acid, a carbohydrate, and a lipid.
I6. The method of I5, wherein the protein is selected from the group consisting of an antibody, a receptor, and an enzyme.
I7. The method of I5 or I6, wherein the protein comprises a disordered domain.
I8. The method of I7, wherein the protein does not comprise a structured domain.
I9. The method of I5, wherein the peptide is a ligand.
I10. The method of any one of I-I3, wherein the target molecule is a nanomaterial.
I11. The method of any one of I-I3, wherein the target molecule is a synthetic polymer.
I12. The method of any one of I-I11, wherein the target molecule is labeled.
I13. The method of any one of I-I12, wherein the target molecule is fluorescently labeled.
I14. The method of any one of I-I13, wherein the test solution comprises a chaotropic agent.
I15. The method of I14, wherein the chaotropic agent is urea.
I16. The method of any one of I-I15, wherein the test solution comprises a viscosity agent.
I17. The method of I16, wherein the viscosity agent is glycerol.
I18. The method of any one of I-I17, wherein the test solution comprises a gradient.
I19. The method of I18, wherein the gradient is a temperature gradient, a chemical gradient, or a combination thereof.
I20. The method of any one of I-I19, wherein the test solution comprises at least two phases, or a combination thereof.
I21. The method of any one of I-I20, wherein the interaction between the target protein and the test solution results in a conformational change in the target molecule.
I22. The method of any one of I-I21, wherein the property of the test solution is Ph, an ion concentration, an organic molecule concentration, or a viscoelastic property.
I23. The method of I22, wherein the viscoelastic property is viscosity.
I24. The method of any one of I-I23, wherein the test solution is a biological sample taken from a subject.
I25. The method of any one of I-I24, wherein the property of the test solution is used to diagnose a disease in the subject.
I26. The method of any one of I-I25, wherein the disease is cancer.
I27. The method of any one of I-I26, wherein comparing the target molecule's movement obtained in (b) with a reference target molecule's movement further comprises comparing the duration of the target molecule's movement change with the duration of the reference target molecule's movement change.
I28. The method of any one of I-I27, wherein comparing the target molecule's movement obtained in (b) with a reference target molecule's movement further comprises comparing the reversibility of the target molecule's movement change with the reversibility of the reference target molecule's movement change.
I29. The method of any one of I-I28, wherein the interaction between the target molecule and test solution is reversible.
I30. The method of any one of I-I29, wherein the interaction between the target molecule and test solution is irreversible.
I31. The method of any one of I-I30, wherein the target molecule and/or the reference target molecule is a component of a mixture composed of a bacterial extract, cell extract, tissue extract, plant extract, or animal extract.
I32. The method of I31, wherein the bacterial extract, cell extract, tissue extract, plant extract, or animal extract is a lysate.
I33. The method of any one of I-I32, wherein the target molecule and/or the reference target molecule is a component of a mixture composed of serum, blood, and other biological samples.
I34. The method of any one of I-I33, wherein the target molecule and/or the reference target molecule is a component of a mixture composed of buffers and glycerol.
I35. The method of any one of I-I34, wherein the first target molecule, second target molecule, and/or test molecule is labeled with a fluorescent protein.
I36. The method of any one of I-I35, wherein the first target molecule, second target molecule, and/or test molecule is labeled by conjugation of a synthetic nanomaterial or polymer.
J. The method of any one of A-136, further comprising time-correlated single-photon counting.
J1. The method of any one of A-J, further comprising step-scan pump-probe spectromicroscopy.
J2. The method of any one of A-J1, wherein the target molecule and/or test molecule is labeled with an ultra-long lifetime fluorophore.
J3. The method of any one of A-J2, further comprising advanced optical techniques.
J4. The method of any one of A-J3, further comprising wobble anisotropy and/or polarization filtering.
J5. The method of any one of A-J4, wherein the change in movement is calculated as a change in the polarization of conjugated fluorescent labels and wherein determining the change in the polarization of conjugated fluorescent labels comprises time-correlated single-photon counting.
J6. The method of any one of A-J5, wherein the change in movement is calculated as a change in the polarization of conjugated fluorescent labels and wherein determining the change in the polarization of conjugated fluorescent labels comprises step-scan pump-probe spectromicroscopy.
J7. The method of any one of A-J6, wherein the target molecule and/or test molecule is labeled with an ultra-long lifetime fluorophore.
J8. The method of any one of A-J7, wherein the change in movement is calculated as a change in the polarization of conjugated fluorescent labels and wherein determining the change in the polarization of conjugated fluorescent labels comprises advanced optical techniques.
J9. The method of any one of A-J8, wherein the change in movement is calculated as a change in the polarization of conjugated fluorescent labels and wherein determining the change in the polarization of conjugated fluorescent labels comprises wobble anisotropy and/or polarization filtering.
K. A system for performing the method of any one of A-J9.
K1. The system of K comprising a microfluidic device.

Although the presently disclosed subject matter and its advantages have been described in detail, it should be understood that various changes, substitutions and alterations can be made herein without departing from the spirit and scope of the present disclosure. Moreover, the scope of the present application is not intended to be limited to the particular embodiments of the process, machine, manufacture, and composition of matter, means, methods and steps described in the specification. As one of ordinary skill in the art will readily appreciate from the invention of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Various patents, patent applications, publications, product descriptions, protocols, and sequence accession numbers are cited throughout this application, the contents of which are incorporated by reference in their entirety for all purposes.

What is claimed is:

1. A method of identifying an interaction between a target molecule and a test molecule comprising:
(a) contacting a cell-free sample comprising a plurality of the target molecule with a plurality of the test molecule;
(b) tracking a plurality of the target molecules over time to provide a plurality of spatiotemporal trajectories and/or measurements of rotational motion;
(c) analyzing the plurality of spatiotemporal trajectories and/or measurements of rotational motion to determine the target molecule's movement in the presence of the test molecule; and
(d) comparing the target molecule's movement obtained in (c) with a reference target molecule's movement, wherein the reference target molecule's movement is the movement of the target molecule in the absence of the test molecule;
wherein a change in the target molecule's movement compared to the reference target molecule's movement indicates an interaction between the target molecule and the test molecule.

2. The method of claim 1, wherein the method is a high throughput method.

3. The method of claim 1 or 2, wherein the change in movement is calculated as a change in:
(a) diffusion coefficient obtained from a maximum likelihood estimator;
(b) geometric mean posterior diffusion coefficient;
(c) median of the jump length distribution;
(d) 3rd quartile of the jump length distribution;
(e) median radius of gyration;
(f) mean posterior diffusion coefficient;
(g) mean squared displacement;
(h) median bond angle;
(i) spatiotemporal trajectory length;
(j) anisotropy decay time;
(k) spatial extent of detection;
(l) number and wavelength of conjugated fluorescent labels;
(m) occupancy in different diffusive states obtained from state arrays;
(n) polarization of conjugated fluorescent labels; or
(o) state occupation via inference.

4. The method of any one of claims 1-3, wherein the change in movement is calculated as a change in diffusion coefficient.

5. The method of any one of claims 1-4, wherein an increase or decrease in the target molecule's movement compared to the reference target molecule's movement indicates an interaction between the target molecule and the test molecule.

6. The method of any one of claims 1-5, wherein the interaction between the target molecule and the test molecule is reversible or irreversible.

7. The method of any one of claims 1-6, wherein the duration and/or reversibility of the target molecule's movement change compared to the duration and/or reversibility of the reference target molecule's movement change indicates an interaction between the target molecule and the test molecule.

8. The method of any one of claims 1-7, wherein the target molecule and/or test molecule is a component of a mixture comprising a bacterial extract, cell extract, tissue extract, plant extract, or animal extract.

9. The method of claim 8, wherein the bacterial extract, cell extract, tissue extract, plant extract, or animal extract is a lysate.

10. The method of any one of claims 1-9, wherein the target molecule and/or test molecule is a component of a mixture comprising serum, blood, and/or other biological samples.

11. The method of any one of claims 1-10, wherein the target molecule and/or test molecule is a component of a mixture comprising a buffer and glycerol.

12. The method of any one of claims 1-11, wherein the target molecule and/or the test molecule is an organic molecule less than 1 KDa.

13. The method of any one of claims 1-12, wherein the target molecule and/or the test molecule is selected from the group consisting of a peptide, a protein, a nucleic acid, a carbohydrate, and a lipid.

14. The method of claim 13, wherein the protein is selected from the group consisting of an antibody, a receptor, and an enzyme.

15. The method of claim 13 or 14, wherein the protein comprises a disordered domain and/or does not comprise a structured domain.

16. The method of claim 13, wherein the peptide is a ligand.

17. The method of any one of claims 1-11, wherein the target molecule and/or the test molecule is a nanomaterial or a synthetic polymer.

18. The method of any one of claims 1-17, wherein the target molecule and/or the test molecule is labeled.

19. The method of any one of claims 1-18, wherein the target molecule and/or the test molecule is fluorescently labeled.

20. The method of any one of claims 1-19, wherein the target molecule and/or the test molecule is labeled with a fluorescent protein.

21. The method of any one of claims 1-20, wherein the plurality of target molecules and/or the plurality of test molecules are conjugated to a plurality of fluorescent labels.

22. The method of any one of claims 1-21, wherein the target molecule and/or the test molecule is labeled by conjugation to a synthetic nanomaterial or polymer.

23. The method of any one of claims 1-22, wherein the change in the target molecule's movement compared to the reference target molecule's movement indicates a direct or indirect interaction between the target molecule and the test molecule.

24. The method of any one of claims 1-23, wherein the interaction between the target molecule and the test molecule results in a conformational change in the target molecule, an increase in the mass of the target molecule, a change in the temperature stability of the target molecule, and/or an oligomerization of the target molecule.

25. The method of any one of claims 1-24, wherein the cell-free sample comprises a solution.

26. The method of claim 25, wherein the solution comprises a chaotropic agent, a carrier, a viscosity agent, or a combination thereof.

27. The method of claim 26, wherein the viscosity agent comprises glycerol.

28. The method of claim 27, wherein the solution comprises at least about 30% glycerol.

29. The method of any one of claims 25-28, wherein the solution comprises a temperature gradient, a chemical gradient, at least two phases, or a combination thereof.

30. The method of any one of claims 1-29, wherein the cell-free sample has a volume of about 0.1 μl to about 100 μl.

31. The method of any one of claims 1-30, wherein the change in movement is measured as a change in the diffusion coefficient of the target molecule as compared to the reference target molecule's diffusion coefficient, and the change is at least about 0.001%, at least about 0.005%, at least 0.01%, at least about 0.05%, at least about 0.1%, at least about 0.5%, at least about 1%, at least about 1.5%, at least about 2% or at least about 3%, at least about 4%, at least about 5%, at least about 6%, at least about 7%, at least about 8%, at least about 9% or at least about 10%.

32. A method for identifying an interaction between a test molecule and a target protein comprising a disordered domain comprising:
(a) contacting a cell-free sample comprising a plurality of the target protein with a plurality of the test molecule;
(b) tracking a plurality of the target protein over time to provide a plurality of spatiotemporal trajectories and/or measurements of rotational motion;
(c) analyzing the plurality of spatiotemporal trajectories and/or measurements of rotational motion to determine the target protein's movement in the presence of the test molecule; and
(d) comparing the target protein's movement obtained in (c) with a reference target protein's movement, wherein the reference target protein's movement is the movement of the target protein in the absence of the test molecule;
wherein a change in the movement of the target protein comprising the disordered domain compared to the reference target protein's movement indicates an interaction between the target protein comprising the disordered domain and the test molecule.

33. The method of claim 32, wherein the method is a high throughput method.

34. The method of claim 32 or 33, wherein the change in movement is calculated as a change in:
(a) diffusion coefficient obtained from a maximum likelihood estimator;
(b) geometric mean posterior diffusion coefficient;
(c) median of the jump length distribution;
(d) 3rd quartile of the jump length distribution;
(e) median radius of gyration;
(f) mean posterior diffusion coefficient;
(g) mean squared displacement;
(h) median bond angle;
(i) spatiotemporal trajectory length;
(j) anisotropy decay time;
(k) spatial extent of detection;
(l) number and wavelength of conjugated fluorescent labels;
(m) occupancy in different diffusive states obtained from state arrays;
(n) polarization of conjugated fluorescent labels; or
(o) state occupation via inference.

35. The method of any one of claims 32-34, wherein the change in movement is calculated as a change in diffusion coefficient.

36. The method of any one of claims 32-35, wherein an increase or decrease in the movement of the target protein comprising the disordered domain compared to the reference target protein's movement indicates an interaction between the target protein comprising the disordered domain and the test molecule.

37. The method of any one of claims 32-36, wherein the interaction between the target protein comprising the disordered domain and the test molecule is reversible or irreversible.

38. The method of any one of claims 32-37, wherein the duration and/or reversibility of the movement change of the target protein comprising the disordered domain compared to the duration and/or reversibility of the reference target protein's movement change indicates an interaction between the target molecule and the test molecule.

39. The method of any one of claims 32-38, wherein the target protein comprising the disordered domain and/or the test molecule is a component of a mixture comprising a bacterial extract, cell extract, tissue extract, plant extract, or animal extract.

40. The method of claim 39, wherein the bacterial extract, cell extract, tissue extract, plant extract, or animal extract is a lysate.

41. The method of any one of claims 32-40, wherein the target protein comprising the disordered domain and/or the test molecule is a component of a mixture composed of serum, blood, and other biological samples.

42. The method of any one of claims 32-41, wherein the target protein comprising the disordered domain and/or the test molecule is a component of a mixture comprising a buffer and glycerol.

43. The method of any one of claims 32-42, wherein the target protein comprising the disordered domain is selected from the group consisting of an antibody, a receptor, and an enzyme.

44. The method of any one of claims 32-43, wherein the target protein comprising the disordered domain does not comprise a structured domain.

45. The method of any one of claims 32-44, wherein the test molecule is an organic molecule less than 1 KDa.

46. The method of any one of claims 32-45, wherein the test molecule is selected from the group consisting of a peptide, a protein, a nucleic acid, a carbohydrate, and a lipid.

47. The method of claim 46, wherein the protein is selected from the group consisting of an antibody, a receptor, and an enzyme.

48. The method of claim 46 or 47, wherein the protein comprises a disordered domain and/or does not comprise a structured domain.

49. The method of claim 46, wherein the peptide is a ligand.

50. The method of any one of claims 32-49, wherein the target protein comprising the disordered domain and/or the test molecule is labeled.

51. The method of any one of claims 32-50, wherein the target protein comprising the disordered domain and/or the test molecule is fluorescently labeled.

52. The method of any one of claims 32-51, wherein the target protein and/or the test molecule is labeled with a fluorescent protein.

53. The method of any one of claims 32-52, wherein the target protein and/or the test molecule is labeled by conjugation of a synthetic nanomaterial or polymer.

54. The method of any one of claims 32-53, wherein the change in the movement of the target protein comprising the disordered domain compared to the reference target protein's movement indicates a direct or indirect interaction between the target protein comprising the disordered domain and the test molecule.

55. The method of any one of claims 32-54, wherein the interaction between the target protein comprising the disordered domain and the test molecule results in a conformational change in the target protein, a change in the temperature stability of the target protein, an increase in the mass of the target protein, and/or an oligomerization of the target protein.

56. The method of any one of claims 32-55, wherein the cell-free sample comprises a solution.

57. The method of claim 56, wherein the solution comprises a chaotropic agent, a carrier, a viscosity agent, or a combination thereof.

58. The method of claim 57, wherein the viscosity agent comprises glycerol.

59. The method of claim 58, wherein the solution comprises at least about 30% glycerol.

60. The method of any one of claims 56-59, wherein the solution comprises a temperature gradient, a chemical gradient, at least two phases, or a combination thereof.

61. The method of any one of claims 32-60, wherein the cell-free sample has a volume of about 0.1 μl to about 100 μl.

62. The method of any one of claims 32-61, wherein the change in movement is measured as a change in the diffusion coefficient of the target protein as compared to the reference target protein's diffusion coefficient, and the change is at least about 0.001%, at least about 0.005%, at least 0.01%, at least about 0.05%, at least about 0.1%, at least about 0.5%, at least about 1%, at least about 1.5%, at least about 2% or at least about 3%, at least about 4%, at least about 5%, at least about 6%, at least about 7%, at least about 8%, at least about 9% or at least about 10%.

63. A method for analyzing a target molecule in a test solution comprising:
(a) tracking a plurality of the target molecules over time in the test solution to provide a plurality of spatiotemporal trajectories and/or measurements of rotational motion;
(b) analyzing the plurality of spatiotemporal trajectories and/or measurements of rotational motion to determine the target molecule's movement in the test solution; and
(c) comparing the target molecule's movement obtained in (b) with a reference target molecule's movement, wherein the reference target molecule's movement is the movement of the target molecule in a reference solution;
wherein: (i) a change in the target molecule's movement compared to the reference target molecule's movement indicates that the target molecule is affected by the test solution; or (ii) no change in the target molecule's movement compared to the reference target molecule's movement indicates that the target molecule is not affected by the test solution.

64. The method of claim 63, wherein the method is a high throughput method.

65. The method of claim 63 or 64, wherein the change in movement is calculated as a change in:
(a) diffusion coefficient obtained from a maximum likelihood estimator;
(b) geometric mean posterior diffusion coefficient;
(c) median of the jump length distribution;
(d) 3rd quartile of the jump length distribution;
(e) median radius of gyration;
(f) mean posterior diffusion coefficient;
(g) mean squared displacement;
(g) median bond angle;
(i) spatiotemporal trajectory length;
(j) anisotropy decay time;
(k) spatial extent of detection;
(l) number and wavelength of conjugated fluorescent labels;
(m) occupancy in different diffusive states obtained from state arrays;
(n) polarization of conjugated fluorescent labels; or
(o) state occupation via inference.

66. The method of any one of claims 63-65, wherein the change in movement is calculated as a change in diffusion coefficient.

67. The method of any one of claims 63-66, wherein the duration and/or reversibility of the target molecule's movement change compared to the duration and/or reversibility of the reference target molecule's movement change indicates that the target molecule is affected by the test solution.

68. The method of any one of claims 63-67, wherein the target molecule and/or the reference target molecule is a component of a mixture composed of a bacterial extract, cell extract, tissue extract, plant extract, or animal extract.

69. The method of claim 68, wherein the bacterial extract, cell extract, tissue extract, plant extract, or animal extract is a lysate.

70. The method of any one of claims 63-69, wherein the target molecule and/or the reference target molecule is a component of a mixture composed of serum, blood, and other biological samples.

71. The method of any one of claims 63-70, wherein the target molecule and/or the reference target molecule is a component of a mixture comprising a buffer and glycerol.

72. The method of any one of claims 63-71, wherein the target molecule and/or the reference target molecule is an organic molecule less than 1 KDa.

73. The method of any one of claims 63-72, wherein the target molecule and/or the reference target molecule is selected from the group consisting of a peptide, a protein, a nucleic acid, a carbohydrate, and a lipid.

74. The method of claim 73, wherein the protein is selected from the group consisting of an antibody, a receptor, and an enzyme.

75. The method of claim 73 or 74, wherein the protein comprises a disordered domain and/or does not comprise a structured domain.

76. The method of claim 73, wherein the peptide is a ligand.

77. The method of any one of claims 63-72, wherein the target molecule and/or the reference target molecule is a nanomaterial or a synthetic polymer.

78. The method of any one of claims 63-77, wherein the target molecule and/or the reference target molecule is labeled.

79. The method of any one of claims 63-78, wherein the target molecule and/or the reference target molecule is fluorescently labeled.

80. The method of any one of claims 63-79, wherein the target molecule and/or the reference target molecule is labeled with a fluorescent protein.

81. The method of any one of claims 63-80, wherein the target molecule and/or the reference target molecule is labeled by conjugation to a synthetic nanomaterial or polymer.

82. The method of any one of claims 63-81, wherein the test solution comprises a chaotropic agent and/or a viscosity agent.

83. The method of claim 82, wherein the chaotropic agent is urea.

84. The method of claim 82 or 83, wherein the viscosity agent is glycerol.

85. The method of any one of claims 63-84, wherein the test solution comprises a gradient.

86. The method of claim 85, wherein the gradient is a temperature gradient, a chemical gradient, or a combination thereof.

87. The method of any one of claims 63-86, wherein the test solution comprises at least two phases, or a combination thereof.

88. The method of any one of claims 63-87, wherein the interaction between the target molecule and the test solution results in a conformational change in the target molecule.

89. The method of any one of claims 63-88, wherein the interaction between the target molecule and the test solution is reversible or irreversible.

90. A method for determining the form of a target molecule comprising:
(a) tracking a plurality of the target molecules over time in a cell-free sample to provide a plurality of spatiotemporal trajectories and/or measurements of rotational motion;
(b) analyzing the plurality of spatiotemporal trajectories and/or measurements of rotational motion to determine the target molecule's movement; and
(c) comparing the target molecule's movement obtained in (b) with a reference target molecule's movement, wherein the reference target molecule's movement is the movement of one form of the target molecule;
wherein: (i) a change in the target molecule's movement compared to the reference target molecule's movement indicates that the target molecule is of a different form than the reference target molecule; or (ii) no change in the target molecule's movement compared to the reference target molecule's movement indicates that the target molecule is of the same form as the reference target molecule.

91. The method of claim 90, wherein the method is a high throughput method.

92. The method of claim 90 or 91, wherein the change in movement is calculated as a change in:
(a) diffusion coefficient obtained from a maximum likelihood estimator;
(b) geometric mean posterior diffusion coefficient;
(c) median of the jump length distribution;
(d) 3rd quartile of the jump length distribution;
(e) median radius of gyration;
(f) mean posterior diffusion coefficient;
(g) mean squared displacement;
(h) median bond angle;
(i) spatiotemporal trajectory length;
(j) anisotropy decay time;
(k) spatial extent of detection;
(l) number and wavelength of conjugated fluorescent labels;
(m) occupancy in different diffusive states obtained from state arrays;
(n) polarization of conjugated fluorescent labels; or
(o) state occupation via inference.

93. The method of any one of claims 90-92, wherein the change in movement is calculated as a change in diffusion coefficient.

94. The method of any one of claims 90-93, wherein the duration and/or reversibility of the target molecule's movement change compared to the duration and/or reversibility of the reference target molecule's movement change indicates that the target molecule is of a different form than the reference target molecule.

95. The method of any one of claims 90-94, wherein the target molecule and/or the reference target molecule is a component of a mixture composed of a bacterial extract, cell extract, tissue extract, plant extract, or animal extract.

96. The method of claim 95, wherein the bacterial extract, cell extract, tissue extract, plant extract, or animal extract is a lysate.

97. The method of any one of claims 90-96, wherein the target molecule and/or the reference target molecule is a component of a mixture composed of serum, blood, and other biological samples.

98. The method of any one of claims 90-97, wherein the target molecule and/or the reference target molecule is a component of a mixture comprising a buffer and glycerol.

99. The method of any one of claims 90-98, wherein the target molecule and/or the reference target molecule is an organic molecule less than 1 KDa.

100. The method of any one of claims 90-99, wherein the target molecule and/or the reference target molecule is selected from the group consisting of a peptide, a protein, a nucleic acid, a carbohydrate, and a lipid.

101. The method of claim 100, wherein the protein is selected from the group consisting of an antibody, a receptor, and an enzyme.

102. The method of claim 100 or 101, wherein the protein comprises a disordered domain and/or does not comprise a structured domain.

103. The method of claim 100, wherein the peptide is a ligand.

104. The method of any one of claims 90-98, wherein the target molecule and/or the reference target molecule is a nanomaterial or a synthetic polymer.

105. The method of any one of claims 90-104, wherein the target molecule and/or the reference target molecule is labeled.

106. The method of any one of claims 90-105, wherein the target molecule and/or the reference target molecule is fluorescently labeled.

107. The method of any one of claims 90-106, wherein the target molecule and/or the reference target molecule is labeled with a fluorescent protein.

108. The method of any one of claims 90-107, wherein the target molecule and/or the reference target molecule is labeled by conjugation of a synthetic nanomaterial or polymer.

109. The method of any one of claims 90-108, wherein the cell-free sample comprises a solution.

110. The method of claim 109, wherein the solution comprises a chaotropic agent, a carrier, a viscosity agent, or a combination thereof.

111. The method of claim 110, wherein the chaotropic agent is urea.

112. The method of claim 110 or 111, wherein the viscosity agent comprises glycerol.

113. The method of claim 112, wherein the solution comprises at least about 30% glycerol.

114. The method of any one of claims 109-113, wherein the solution comprises a temperature gradient, a chemical gradient, at least two phases, or a combination thereof.

115. The method of any one of claims 90-114, wherein the cell-free sample has a volume of about 0.1 μl to about 100 μl.

116. The method of any one of claims 90-115, wherein the change in movement is measured as a change in the diffusion coefficient of the target molecule as compared to the reference target molecule's diffusion coefficient, and the change is at least about 0.001%, at least about 0.005%, at least 0.01%, at least about 0.05%, at least about 0.1%, at least about 0.5%, at least about 1%, at least about 1.5%, at least about 2% or at least about 3%, at least about 4%, at least about 5%, at least about 6%, at least about 7%, at least about 8%, at least about 9% or at least about 10%.

117. The method of any one of claims 90-116, wherein the form of the target molecule is a post-translationally modified form of the target molecule, a wild type form of the target molecule, or a mutant form of the target molecule.

118. A method for identifying a test molecule that can distinguish between at least two target molecules comprising:
(a) contacting a cell-free sample comprising a plurality of a first target molecule with a plurality of the test molecule;
(b) tracking a plurality of the first target molecules over time to provide a plurality of spatiotemporal trajectories and/or measurements of rotational motion;
(c) analyzing the plurality of spatiotemporal trajectories and/or measurements of rotational motion to determine the first target molecule's movement in the presence of the test molecule;
(d) contacting a cell-free sample comprising a plurality of a second target molecule with a plurality of the test molecule;
(e) tracking a plurality of the second target molecules over time to provide a plurality of spatiotemporal trajectories and/or measurements of rotational motion;
(f) analyzing the plurality of spatiotemporal trajectories and/or measurements of rotational motion to determine the second target molecule's movement in the presence of the test molecule; and
(g) comparing the first and second target molecules' movement obtained in (c) and (f);
wherein a change in the movement of the first and second target molecules indicates that the test molecule can distinguish between the two target molecules.

119. The method of claim 118, wherein the method is a high throughput method.

120. The method of claim 118 or 119, wherein the change in movement is calculated as a change in:
(a) diffusion coefficient obtained from a maximum likelihood estimator;
(b) geometric mean posterior diffusion coefficient;
(c) median of the jump length distribution;
(d) 3rd quartile of the jump length distribution;
(e) median radius of gyration;
(f) mean posterior diffusion coefficient;
(g) mean squared displacement;
(h) median bond angle;
(i) spatiotemporal trajectory length;
(j) anisotropy decay time;
(k) spatial extent of detection;
(l) number and wavelength of conjugated fluorescent labels;
(m) occupancy in different diffusive states obtained from state arrays;
(n) polarization of conjugated fluorescent labels; or
(o) state occupation via inference.

121. The method of any one of claims 118-120, wherein the change in movement is calculated as a change in diffusion coefficient.

122. The method of any one of claims 118-121, wherein the duration and/or reversibility of the movement change of the first and second target molecules indicates that the test molecule can distinguish between the two target molecules.

123. The method of any one of claims 118-122, wherein the first target molecule, the second target molecule and/or the test molecule is a component of a mixture composed of a bacterial extract, cell extract, tissue extract, plant extract, or animal extract.

124. The method of claim 123, wherein the bacterial extract, cell extract, tissue extract, plant extract, or animal extract is a lysate.

125. The method of any one of claims 118-124, wherein the first target molecule, the second target molecule, and/or the test molecule is a component of a mixture composed of serum, blood, and other biological samples.

126. The method of any one of claims 118-125, wherein the first target molecule, the second target molecule, and/or the test molecule is a component of a mixture comprising a buffer and glycerol.

127. The method of any one of claims 118-126, wherein the first target molecule, the second target molecule, and/or the test molecule is an organic molecule less than 1 KDa.

128. The method of any one of claims 118-127, wherein the first target molecule, the second target molecule, and/or the test molecule is selected from the group consisting of a peptide, a protein, a nucleic acid, a carbohydrate, and a lipid.

129. The method of claim 128, wherein the protein is selected from the group consisting of an antibody, a receptor, and an enzyme.

130. The method of claim 128 or 129, wherein the protein comprises a disordered domain and/or does not comprise a structured domain.

131. The method of claim 128, wherein the peptide is a ligand.

132. The method of any one of claims 118-126, wherein the first target molecule, the second target molecule, and/or the test molecule is a nanomaterial or a synthetic polymer.

133. The method of any one of claims 118-132, wherein the first target molecule, the second target molecule, and/or the test molecule is labeled.

134. The method of any one of claims 118-133, wherein the first target molecule, the second target molecule, and/or the test molecule is fluorescently labeled.

135. The method of any one of claims 118-134, wherein first target molecule, the second target molecule and/or the test molecule is labeled with a fluorescent protein.

136. The method of any one of claims 118-135, wherein first target molecule, the second target molecule and/or the test molecule is labeled by conjugation of a synthetic nanomaterial or polymer.

137. The method of any one of claims 118-136, wherein the first and second target molecules are distinguished by a difference in their fluorescent labels.

138. The method of any one of claims 118-137, wherein the first target molecule and the second target molecule are related target molecules.

139. The method of claim 138, wherein at least one of the target molecules is a wild type form of the target molecule; wherein at least one of the target molecules is a mutant form of the target molecule; and/or wherein at least one of the target molecules is a post-translationally modified form of the target molecule.

140. The method of any one of claims 118-138, wherein the first target molecule and the second target molecule are the same or different conformational states of the target molecule; wherein the first target molecule and the second target molecule are homologs, orthologs or paralogs; and/or wherein the first target molecule and the second target molecule are unrelated target molecules.

141. The method of any one of claims 118-140, further comprising: contacting a cell-free sample comprising a plurality of a third target molecule with a plurality of the test molecule; tracking a plurality of the third target molecules over time to provide a third plurality of spatiotemporal trajectories and/or measurements of rotational motion; analyzing the third plurality of spatiotemporal trajectories and/or measurements of rotational motion to determine the movement of the third target molecules in the presence of the test molecule; and comparing the third target molecule's movement to the movements obtained in (c) and (f); wherein a change in the movement of the third target molecule relative to the first and second target molecules indicates that the test molecule can distinguish between the three target molecules.

142. A method for identifying one or more test molecules that induce a conformational change in a target molecule comprising:
(a) contacting cell-free samples comprising a plurality of the target molecule with a plurality of test molecules, where each sample is contacted with a different test molecule;
(b) tracking a plurality of the target molecules in each sample over time to provide a plurality of spatiotemporal trajectories and/or measurements of rotational motion;
(c) analyzing the plurality of spatiotemporal trajectories and/or measurements of rotational motion to determine the target molecule's movement in the presence of each of the different test molecules; and
(d) comparing the target molecule's movement obtained in (c) with a reference target molecule's movement, wherein the reference target molecule's movement is the movement of the target molecule in the presence of a test molecule that does not induce a conformational change in the target molecule;
wherein a change in the target molecule's movement compared to the reference target molecule's movement indicates an interaction between the target molecule and the one or more test molecules that induce a conformational change in the target molecule.

143. The method of claim 142, wherein the method is a high throughput method.

144. The method of claim 142 or 143, wherein the change in movement is calculated as a change in:
(a) diffusion coefficient obtained from a maximum likelihood estimator;
(b) geometric mean posterior diffusion coefficient;
(c) the median of the jump length distribution;
(b) 3rd quartile of the jump length distribution;
(c) median radius of gyration;
(d) mean posterior diffusion coefficient;
(f) mean squared displacement;
(g) median bond angle;
(i) the spatiotemporal trajectory length;
(j) anisotropy decay time;
(k) spatial extent of detection;
(l) number and wavelength of conjugated fluorescent labels;
(m) occupancy in different diffusive states obtained from state arrays;
(n) polarization of conjugated fluorescent labels; or
(o) state occupation via inference.

145. The method of any one of claims 142-144, wherein the change in movement is calculated as a change in diffusion coefficient.

146. The method of any one of claims 142-145, wherein the conformational change in the target molecule is associated with a change in the target molecule's temperature stability.

147. The method of any one of claims 142-146, wherein the interaction between the target molecule and the one or more test molecules is reversible or irreversible.

148. The method of any one of claims 142-147, wherein the duration and/or reversibility of the target molecule's movement change compared to the duration and/or reversibility of the reference target molecule's movement change indicates an interaction between the target molecule and the one or more test molecules that induce a conformational change in the target molecule.

149. The method of any one of claims 142-148, wherein the target molecule, the reference target molecule and/or the one or more test molecules is a component of a mixture composed of a bacterial extract, cell extract, tissue extract, plant extract, or animal extract.

150. The method of claim 149, wherein the bacterial extract, cell extract, tissue extract, plant extract, or animal extract is a lysate.

151. The method of any one of claims 142-150, wherein the target molecule, the reference target molecule and/or the one or more test molecules is a component of a mixture composed of serum, blood, and other biological samples.

152. The method of any one of claims 142-151, wherein the target molecule, the reference target molecule and/or the one or more test molecules is a component of a mixture comprising a buffer and glycerol.

153. The method of any one of claims 142-152, wherein the target molecule, the reference target molecule and/or the one or more test molecules is an organic molecule less than 1 KDa.

154. The method of any one of claims 142-153, wherein the target molecule, the reference target molecule and/or the one or more test molecules is selected from the group consisting of a peptide, a protein, a nucleic acid, a carbohydrate, and a lipid.

155. The method of claim 154, wherein the protein is selected from the group consisting of an antibody, a receptor, and an enzyme.

156. The method of claim 154 or 155, wherein the protein comprises a disordered domain and/or does not comprise a structured domain.

157. The method of claim 154, wherein the peptide is a ligand.

158. The method of any one of claims 142-152, wherein the target molecule, the reference target molecule and/or the one or more test molecules is a nanomaterial or a synthetic polymer.

159. The method of any one of claims 142-158, wherein the target molecule, the reference target molecule and/or the one or more test molecules is labeled.

160. The method of any one of claims 142-159, wherein the target molecule, the reference target molecule and/or the one or more test molecules is fluorescently labeled.

161. The method of any one of claims 142-160, wherein the target molecule, the reference target molecule and/or the one or more test molecules is labeled with a fluorescent protein.

162. The method of any one of claims 142-161, wherein the target molecule, the reference target molecule and/or the one or more test molecule is labeled by conjugation of a synthetic nanomaterial or polymer.

163. The method of any one of claims 142-162, wherein the cell-free sample comprises a solution.

164. The method of claim 163, wherein the solution comprises a chaotropic agent, a viscosity agent, or a combination thereof.

165. The method of claim 164, wherein the viscosity agent comprises glycerol.

166. The method of claim 165, wherein the solution comprises at least about 30% glycerol.

167. The method of any one of claims 163-166, wherein the solution comprises a temperature gradient, a chemical gradient, at least two phases, or a combination thereof.

168. The method of any one of claims 142-167, wherein the sample has a volume of about 0.1 μl to about 100 μl.

169. The method of any one of claims 142-168, wherein the change in movement is measured as a change in the diffusion coefficient of the target molecule in the presence of a test molecule and in the presence of a reference test molecule that does not induce a conformational change, and the change is at least about 0.001%, at least about 0.005%, at least 0.01%, at least about 0.05%, at least about 0.1%, at least about 0.5%, at least about 1%, at least about 1.5%, at least about 2% or at least about 3%, at least about 4%, at least about 5%, at least about 6%, at least about 7%, at least about 8%, at least about 9% or at least about 10%.

170. A method for determining a dose response for the interaction of a target molecule and a test molecule comprising:
(a) contacting a plurality of cell-free samples comprising a plurality of the target molecule with the test molecule, where plurality of cell-free samples are contacted with a range of test molecule doses;
(b) tracking a plurality of the target molecules over time in the presence of the range of doses of the test molecule to provide a plurality of spatiotemporal trajectories and/or measurements of rotational motion;
(c) analyzing the plurality of spatiotemporal trajectories and/or measurements of rotational motion of the target molecules in the presence of the range of doses of the test molecule to determine the target molecule's movement at each dose of test molecule;
(d) comparing the target molecule's movement obtained at different test molecule doses in step (c) to determine the dose response of the target molecule to the test molecule.

171. A method for determining a difference in dose response to a test molecule by two target molecules comprising:
(a) contacting a first plurality of cell-free samples comprising a plurality of the first target molecule with the test molecule, where first plurality of cell-free samples are contacted with a range of test molecule doses;
(b) tracking a plurality of the target molecules over time in the presence of the range of doses of the test molecule to provide a first plurality of spatiotemporal trajectories and/or measurements of rotational motion;
(c) analyzing the plurality of spatiotemporal trajectories and/or measurements of rotational motion of the target molecules in the presence of the range of doses of the test molecule to determine the first target molecule's movement at each concentration of test molecule;
(d) comparing the target molecule's movement obtained at different test molecule doses in step (c) to determine the dose response of the target molecule to the test molecule;
(e) repeating steps (a)-(d) with a second target molecule to determine the dose response of the second target molecule to the test molecule; and
(f) comparing the dose response of the first target molecule to the dose response of the second target molecule to determine the difference in the response of the first and second target molecules to the test molecule.

172. The method of claim 170 or 171, wherein the method is a high throughput method.

173. The method of any one of claims 170-172, wherein the movement is calculated as:
(a) diffusion coefficient of the plurality of spatiotemporal trajectories obtained from a maximum likelihood estimator;
(b) geometric mean posterior diffusion coefficient of the plurality of spatiotemporal trajectories;
(c) the median of the jump length distribution of the plurality of spatiotemporal trajectories;
(d) 3rd quartile of the jump length distribution of the plurality of spatiotemporal trajectories;
(e) median radius of gyration of the plurality of spatiotemporal trajectories;
(f) mean posterior diffusion coefficient of the plurality of spatiotemporal trajectories;
(g) mean squared displacement of the plurality of spatiotemporal trajectories;
(h) median bond angle of the plurality of spatiotemporal trajectories;
(i) the spatiotemporal trajectory length of the plurality of spatiotemporal trajectories;
(j) anisotropy decay time;
(k) spatial extent of detection;
(l) number and wavelength of conjugated fluorescent labels;
(m) occupancy in different diffusive states obtained from state arrays;
(n) polarization of conjugated fluorescent labels; or
(o) state occupation via inference.

174. The method of any one of claims 170-173, wherein the movement is calculated as a diffusion coefficient.

175. The method of any one of claims 170-174, wherein the interaction between the target molecule and the test molecule is reversible or irreversible.

176. The method of any one of claims 170-175, wherein comparing the target molecule's movement obtained at different test molecule doses in step (c) further comprises comparing the duration and/or reversibility of the target molecule's movement changes.

177. The method of any one of claims 170-176, wherein the first target molecule, the second target molecule and/or the test molecule is a component of a mixture composed of a bacterial extract, cell extract, tissue extract, plant extract, or animal extract.

178. The method of claim 177, wherein the bacterial extract, cell extract, tissue extract, plant extract, or animal extract is a lysate.

179. The method of any one of claims 170-178, wherein the first target molecule, the second target molecule, and/or the test molecule is a component of a mixture composed of serum, blood, and other biological samples.

180. The method of any one of claims 170-179, wherein the first target molecule, the second target molecule, and/or the test molecule is a component of a mixture comprising a buffer and glycerol.

181. The method of any one of claims 170-180, wherein the first target molecule, the second target molecule, and/or the test molecule is an organic molecule less than 1 KDa.

182. The method of any one of claims 170-181, wherein the first target molecule, the second target molecule, and/or the test molecule is selected from the group consisting of a peptide, a protein, a nucleic acid, a carbohydrate, and a lipid.

183. The method of claim 182, wherein the protein is selected from the group consisting of an antibody, a receptor, and an enzyme.

184. The method of claim 182 or 183, wherein the protein comprises a disordered domain and/or does not comprise a structured domain.

185. The method of claim 182, wherein the peptide is a ligand.

186. The method of any one of claims 170-180, wherein the first target molecule, the second target molecule, and/or the test molecule is a nanomaterial or a synthetic polymer.

187. The method of any one of claims 170-186, wherein the first target molecule, the second target molecule, and/or the test molecule is labeled.

188. The method of any one of claims 170-187, wherein the first target molecule, the second target molecule, and/or the test molecule is fluorescently labeled.

189. The method of any one of claims 170-188, wherein the first target molecule, the second target molecule and/or the test molecule is labeled with a fluorescent protein.

190. The method of any one of claims 170-189, wherein the first target molecule, the second target molecule and/or the test molecule is labeled by conjugation of a synthetic nanomaterial or polymer.

191. The method of any one of claims 170-190, wherein the first target molecule and the second target molecule are distinguished by a difference in their fluorescent labels.

192. The method of claim 171, wherein the first target molecule and the second target molecule are related target molecules.

193. The method of claim 192, wherein at least one of the target molecules is a wild type form of the target molecule; wherein at least one of the target molecules is a mutant form of the target molecule; and/or wherein at least one of the target molecules is a post-translationally modified form of the target molecule.

194. The method of any one of claims 171-192, wherein the first target molecule and the second target molecule are the same or different conformational states of the target molecule; wherein the first target molecule and the second target molecule are homologs, orthologs or paralogs; and/or wherein the first target molecule and the second target molecule are unrelated target molecules.

195. The method of claim 171, wherein first and second cell-free samples comprises a solution that comprises a chaotropic agent a carrier, a viscosity agent, or a combination thereof.

196. The method of claim 195, wherein the viscosity agent comprises glycerol.

197. The method of claim 196, wherein the solution comprises at least about 30% glycerol.

198. The method of any one of claims 195-197, wherein the solution comprises a temperature gradient, a chemical gradient, at least two phases, or a combination thereof.

199. The method of any one of claims 170-198, wherein three or more target molecules are analyzed.

200. A method for identifying a test molecule that can distinguish between at least two target molecules comprising:
(a) contacting a cell-free sample comprising a plurality of a first target molecule and a plurality of a second target molecule with a plurality of the test molecule;
(b) tracking a plurality of the first target molecules over time to provide a plurality of spatiotemporal trajectories and/or measurements of rotational motion;
(c) analyzing the plurality of spatiotemporal trajectories and/or measurements of rotational motion to determine the first target molecule's movement in the presence of the test molecule;
(d) tracking a plurality of the second target molecules over time to provide a plurality of spatiotemporal trajectories and/or measurements of rotational motion;
(e) analyzing the plurality of spatiotemporal trajectories and/or measurements of rotational motion to determine the second target molecule's movement in the presence of the test molecule; and
(f) comparing the first and second target molecules' movement obtained in (c) and (f);
wherein a change in the movement of the first and second target molecules indicates that the test molecule can distinguish between the two target molecules.

201. The method of claim 200, wherein the method is a high throughput method.

202. The method of claim 200 or 201, wherein the change in movement is calculated as a change in:
(a) diffusion coefficient obtained from a maximum likelihood estimator;
(b) geometric mean posterior diffusion coefficient;
(c) median of the jump length distribution;
(d) 3rd quartile of the jump length distribution;
(e) median radius of gyration;
(f) mean posterior diffusion coefficient;
(g) mean squared displacement;
(h) median bond angle;
(i) spatiotemporal trajectory length;
(j) anisotropy decay time;
(k) spatial extent of detection;
(l) number and wavelength of conjugated fluorescent labels;
(m) occupancy in different diffusive states obtained from state arrays;
(n) polarization of conjugated fluorescent labels; or
(o) state occupation via inference.

203. The method of any one of claims 200-202, wherein the change in movement is calculated as a change in diffusion coefficient.

204. The method of any one of claims 200-203, wherein the duration and/or reversibility of the first molecule's movement change compared to the duration and/or reversibility of the second molecule's movement change indicates that the test molecule can distinguish between the two target molecules.

205. The method of any one of claims 200-204, wherein the first target molecule, the second target molecule and/or the test molecule is a component of a mixture composed of a bacterial extract, cell extract, tissue extract, plant extract, or animal extract.

206. The method of claim 205, wherein the bacterial extract, cell extract, tissue extract, plant extract, or animal extract is a lysate.

207. The method of any one of claims 200-206, wherein the first target molecule, the second target molecule, and/or the test molecule is a component of a mixture composed of serum, blood, and other biological samples.

208. The method of any one of claims 200-207, wherein the first target molecule, the second target molecule, and/or the test molecule is a component of a mixture comprising a buffer and glycerol.

209. The method of any one of claims 200-208, wherein the first target molecule, the second target molecule, and/or the test molecule is an organic molecule less than 1 KDa.

210. The method of any one of claims 200-209, wherein the first target molecule, the second target molecule, and/or the test molecule is selected from the group consisting of a peptide, a protein, a nucleic acid, a carbohydrate, and a lipid.

211. The method of claim 210, wherein the protein is selected from the group consisting of an antibody, a receptor, and an enzyme.

212. The method of claim 210 or 211, wherein the protein comprises a disordered domain and/or does not comprise a structured domain.

213. The method of claim 210, wherein the peptide is a ligand.

214. The method of any one of claims 200-208, wherein the first target molecule, the second target molecule, and/or the test molecule is a nanomaterial or a synthetic polymer.

215. The method of any one of claims 200-214, wherein the first target molecule, the second target molecule, and/or the test molecule is labeled.

216. The method of any one of claims 200-215, wherein the first target molecule, the second target molecule, and/or the test molecule is fluorescently labeled.

217. The method of any one of claims 200-216, wherein the first target molecule, the second target molecule, and/or the test molecule is labeled with a fluorescent protein.

218. The method of any one of claims 200-217, wherein the first target molecule, the second target molecule, and/or the test molecule is labeled by conjugation of a synthetic nanomaterial or polymer.

219. The method of any one of claims 200-218, wherein the first target molecule and the second target molecule are related target molecules.

220. The method of claim 219, wherein at least one of the target molecules is a wild type form of the target molecule; wherein at least one of the target molecules is a mutant form of the target molecule; and/or wherein at least one of the target molecules is a post-translationally modified form of the target molecule.

221. The method of any one of claims 200-219, wherein the first target molecule and the second target molecule are the same or different conformational states of the target molecule; wherein the first target molecule and the second target molecule are homologs, orthologs or paralogs; and/or wherein the first target molecule and the second target molecule are unrelated target molecules.

222. The method of any one of claims 200-221, further comprising: contacting a cell-free sample comprising a plurality of a third target molecule with a plurality of the test molecule; tracking a plurality of the third target molecules over time to provide a third plurality of spatiotemporal trajectories and/or measurements of rotational motion; analyzing the third plurality of spatiotemporal trajectories and/or measurements of rotational motion to determine the movement of the third target molecules in the presence of the test molecule; and comparing the third target molecule's movement to the movements obtained in (c) and (f); wherein a change in the movement of the third target molecule relative to the first and second target molecules indicates that the test molecule can distinguish between the three target molecules.

223. A method for analyzing a test solution comprising a target molecule comprising:
(a) tracking a plurality of a target molecule over time in the test solution to provide a plurality of spatiotemporal trajectories and/or measurements of rotational motion;
(b) analyzing the plurality of spatiotemporal trajectories and/or measurements of rotational motion to determine the target molecule's movement in the test solution; and
(c) comparing the target molecule's movement obtained in (b) with a reference target molecule's movement, wherein the reference target molecule's movement is the movement of the target molecule in a reference solution;
wherein: the comparing of step (c) is used to determine a property of the test solution.

224. The method of claim 223, wherein the method is a high throughput method.

225. The method of claim 223 or 224, wherein the target molecule's movement is calculated as a:
(a) diffusion coefficient obtained from a maximum likelihood estimator;
(b) geometric mean posterior diffusion coefficient;
(c) median of the jump length distribution;
(d) 3rd quartile of the jump length distribution;
(e) median radius of gyration;
(f) mean posterior diffusion coefficient;
(g) mean squared displacement;
(g) median bond angle;
(i) spatiotemporal trajectory length;
(j) anisotropy decay time;
(k) spatial extent of detection;
(l) number and wavelength of conjugated fluorescent labels;
(m) occupancy in different diffusive states obtained from state arrays;
(n) polarization of conjugated fluorescent labels; or
(o) state occupation via inference.

226. The method of any one of claims 223-225, wherein the change in movement is calculated as a change in diffusion coefficient.

227. The method of any one of claims 223-226, wherein comparing the target molecule's movement obtained in (b) with a reference target molecule's movement further comprises comparing the duration and/or reversibility of the target molecule's movement change with the duration of the reference target molecule's movement change.

228. The method of any one of claims 223-227, wherein the target molecule and/or the reference target molecule is a component of a mixture composed of a bacterial extract, cell extract, tissue extract, plant extract, or animal extract.

229. The method of claim 228, wherein the bacterial extract, cell extract, tissue extract, plant extract, or animal extract is a lysate.

230. The method of any one of claims 223-229, wherein the target molecule and/or the reference target molecule is a component of a mixture composed of serum, blood, and other biological samples.

231. The method of any one of claims 223-230, wherein the target molecule and/or the reference target molecule is a component of a mixture comprising a buffer and glycerol.

232. The method of any one of claims 223-231, wherein the target molecule and/or the reference target molecule is an organic molecule less than 1 KDa.

233. The method of any one of claims 223-232, wherein the target molecule and/or the reference target molecule is selected from the group consisting of a peptide, a protein, a nucleic acid, a carbohydrate, and a lipid.

234. The method of claim 233, wherein the protein is selected from the group consisting of an antibody, a receptor, and an enzyme.

235. The method of claim 233 or 234, wherein the protein comprises a disordered domain and/or does not comprise a structured domain.

236. The method of claim 233, wherein the peptide is a ligand.

237. The method of any one of claims 223-231, wherein the target molecule and/or the reference target molecule is a nanomaterial or a synthetic polymer.

238. The method of any one of claims 223-237, wherein the target molecule and/or the reference target molecule is labeled.

239. The method of any one of claims 223-238, wherein the target molecule and/or the reference target molecule is fluorescently labeled.

240. The method of any one of claims 223-239, wherein the test solution comprises a chaotropic agent and/or a viscosity agent.

241. The method of claim 240, wherein the chaotropic agent is urea.

242. The method of claim 240 or 241, wherein the viscosity agent is glycerol.

243. The method of any one of claims 223-242, wherein the test solution comprises a gradient.

244. The method of claim 243, wherein the gradient is a temperature gradient, a chemical gradient, or a combination thereof.

245. The method of any one of claims 223-244, wherein the test solution comprises at least two phases, or a combination thereof.

246. The method of any one of claims 223-245, wherein the interaction between the target molecule and the test solution results in a conformational change in the target molecule.

247. The method of any one of claims 223-246, wherein the interaction between the target molecule and test solution is reversible or irreversible.

248. The method of any one of claims 223-247, wherein the property of the test solution is pH, an ion concentration, an organic molecule concentration, or a viscoelastic property.

249. The method of claim 248, wherein the viscoelastic property is viscosity.

250. The method of any one of claims 223-249, wherein the test solution is a biological sample taken from a subject.

251. The method of any one of claims 223-250, wherein the property of the test solution is used to diagnose a disease in the subject.

252. The method of claim 251, wherein the disease is cancer.

253. The method of any one of claims 1-252, wherein the change in movement is calculated as a change in the polarization of conjugated fluorescent labels and wherein determining the change in the polarization of conjugated fluorescent labels comprises time-correlated single-photon counting.

254. The method of any one of claims 1-253, wherein the change in movement is calculated as a change in the polarization of conjugated fluorescent labels and wherein determining the change in the polarization of conjugated fluorescent labels comprises step-scan pump-probe spectromicroscopy.

255. The method of any one of claims 1-254, wherein the target molecule and/or the test molecule is labeled with an ultra-long lifetime fluorophore.

256. The method of any one of claims 1-255, wherein the change in movement is calculated as a change in the polarization of conjugated fluorescent labels and wherein determining the change in the polarization of conjugated fluorescent labels comprises advanced optical techniques.

257. The method of any one of claims 1-256, wherein the change in movement is calculated as a change in the polarization of conjugated fluorescent labels and wherein determining the change in the polarization of conjugated fluorescent labels comprises wobble anisotropy and/or polarization filtering.

258. A system for performing the method of any one of claims 1-257.

259. The system of claim 258 comprising a microfluidic device.
